(19) United States
(12) Patent Application Publication
(10) Pub. No.: US 2011/0130454 A1
(43) Pub. Date: Jun. 2, 2011

(54) PRODRUGS OF GAMMA-AMINO ACID, ALPHA-2-DELTA LIGANDS, PHARMACEUTICAL COMPOSITIONS AND USES THEREOF

(75) Inventors: Mark A. Gallop, Palo Alto, CA (US); David J. Wustrow, Saratoga, CA (US)

(73) Assignee: XenoPort, Inc., Santa Clara, CA (US)

(21) Appl. No.: 12/953,871

(22) Filed: Nov. 24, 2010

Related U.S. Application Data
(60) Provisional application No. 61/264,193, filed on Nov. 24, 2009.

(51) Int. Cl. A61K 31/27; A61P 25/08; A61P 13/06; A61P 25/06; A61P 19/02; A61P 25/30; C07C 271/22

(52) U.S. Cl. 514/484; 560/160; 514/478; 560/119

(57) ABSTRACT

Prodrugs of alpha-2-delta ligands, pharmaceutical compositions of prodrugs of alpha-2-delta ligands, methods of making prodrugs of alpha-2-delta ligands, and methods of using prodrugs of alpha-2-delta ligands and pharmaceutical compositions of prodrugs of alpha-2-delta ligands to treat various diseases are disclosed.

### CROSS-REFERENCE TO RELATED APPLICATIONS

This application claims priority under 35 U.S.C. §119 to U.S. Provisional Application Ser. No. 61/264,193 filed Nov. 24, 2009, entitled “Prodrugs of Gamma-Amino Acid, Alpha-2-Delta Ligands, Pharmaceutical Compositions and Uses Thereof”, which is incorporated by reference herein in its entirety.

### FIELD

Disclosed herein are prodrugs of alpha-2-delta ligands, pharmaceutical compositions of prodrugs of alpha-2-delta ligands, methods of making prodrugs of alpha-2-delta ligands, and methods of using prodrugs of alpha-2-delta ligands and pharmaceutical compositions of prodrugs of alpha-2-delta ligands to treat various diseases.

### BACKGROUND

Voltage-gated calcium channels are formed by combinations of the pore-forming alpha-1 (α) subunit, and auxiliary alpha-2-delta, beta, and gamma (α₂δ, β and γ, respectively) proteins (Catterall, Annual. Rev. Cell Dev. Biol. 2000, 16, 521-555). The α₂δ protein is known to regulate both the calcium channel density and voltage-dependent kinetics of these calcium channels (Felix et al., J. Neurosci. 1997, 17, 6884-6891; Klugbauer et al., J. Neurosci. 1999, 19, 684-691; Hobom et al., Eur. J. Neurosci. 2000, 12, 1217-1226; and Qin et al., Mol. Pharmacol. 2002, 62, 485-496). The α₂δ protein is encoded by a single gene and post-translationally cleaved to α₂ and δ subunits. The α₂ subunit is a highly glycosylated extracellular protein and is associated with the membrane anchor protein δ by disulfide linkage (Wang et al., Biochem. J. 1999, 342, 313-320; Marais et al., Mol. Pharmacol. 2001, 59, 1243-1248; and Gong et al., J. Membr. Biol. 2001, 164, 35-43). Molecular cloning has revealed four α₂δ subtypes in various species (Qin et al., Mol. Pharmacol. 2002, 62, 485-496).

An alpha-2-delta ligand is a molecule that binds to any subtype of the calcium channel α₂δ subunit. Alpha-2-delta ligands are useful in the treatment of various diseases including epilepsy, pain, depression, anxiety, psychosis, restless legs syndrome, insomnia and other sleep disorders, faintness attacks, hot flashes, hypokinesia, cranial disorders, neurodegenerative disorders, panic, inflammatory disease, gastrointestinal disorders, overactive bladder, and ethanol withdrawal syndrome. Among the known alpha-2-delta ligands are the marketed drugs gabapentin and pregabalin, the latter sold under the trade name LYRICA® and approved for the treatment of epilepsy, post-herpetic neuralgia, diabetic peripheral neuropathy, fibromyalgia, and generalized anxiety disorder.

Alpha-2-delta ligands also include those compounds that are generally or specifically disclosed in the following references: U.S. Pat. No. 4,024,175 and EP 0641330, including 3-methylgabapentin; U.S. Pat. No. 5,563,175, WO 97/33858, WO 97/33859, WO 99/31057, WO 99/31074, WO 97/29101, and WO 02/085839, including [(1R,5R,6S)-6-(aminomethyl)bicyclo[3.2.0]hept-6-yl]acetic acid; WO 99/31075, including 3-{1-aminomethyl-cyclohexylmethyl)-4H-[1,2,4]oxadiazol-5-one, and C-[1-(1H-tetrazol-5-ylmethyl)-cycloheptyl]-methylamine; WO 99/21624, including (3S,4S)-(1-aminomethyl-3,4-dimethyl-cyclopentyl)-acetic acid; WO 01/90052, and WO 01/28978, including (1α,3α,5α)(3-aminomethyl-bicyclo[3.2.0]hept-3-yl)-acetic acid; WO 98/17627 and WO 00/76958, including (3S,5R)-3-aminomethyl-5-methyl-octanoic acid; WO 03/082807, including (3S,5R)-3-amino-5-methyl-heptanoic acid, and (3S,5R)-3-amino-5-methyl-octanoic acid; U.S. Pat. No. 6,642,398, including (3S,5R)-3-aminomethyl-5-methyl-heptanoic acid, (3S,5R)-3-aminomethyl-5-methyl-octanoic acid, (3S,5R)-3-aminomethyl-5-methyl-nonanoic acid, (3S,5R)-3-aminomethyl-5-methyl-decanoic acid, and (3S,5R)-3-aminomethyl-5-methyl-undecanoic acid; US 2004/0132801, including (2S,4S)-4-(3-fluorobenzyl)-pyrrolidine-2-carboxylic acid, (2S,4S)-4-(3-chlorophenoxy)-pyrrolidine-2-carboxylic acid, (2S,4S)-4-(2,3-difluorobenzyl)-pyrrolidine-2-carboxylic acid, (2S,4S)-4-(2,5-difluorobenzyl)-pyrrolidine-2-carboxylic acid, (2S,4S)-4-cyclohexylmethyl-pyrrolidine-2-carboxylic acid, (2S,4S)-4-(3-fluorophenoxymethyl)-pyrrolidine-2-carboxylic acid, (2S,4S)-4-(2,3-difluorophenoxymethyl)-pyrrolidine-2-carboxylic acid, (2S,4S)-4-(2,5-difluorophenoxymethyl)-pyrrolidine-2-carboxylic acid, (2S,4S)-4-(3,6-difluorophenoxymethyl)-pyrrolidine-2-carboxylic acid, and (2S,4S)-4-(3-methoxyphenoxymethyl)-pyrrolidine-2-carboxylic acid; EP 1178034, EP 1201240, WO 99/31074; WO 03/000642; WO 02/22568; WO 02/30871; WO 02/30881; WO 02/100392; WO 02/100347; WO 02/42414; WO 02/32736; WO 02/28881; US 2005/0059654; US 2004/00092522; WO 04/054566; US 2003/019525; US 2005/0124668, including (3R,4R,5R)-3-aminomethyl-4,5-dimethyl-heptanoic acid, (3R,4R,5R)-3-aminomethyl-4,5-dimethyl-octanoic acid, 2-aminomethyl-4,7-dimethyl-octanoic acid, 2-aminomethyl-4-ethyl-6-methyl-heptanoic acid, 2-aminomethyl-4-ethyl-8-methyl-nonanoic acid, 3-amino-3,5-dimethyl-heptanoic acid, 3-amino-3,5-dimethyl-octanoic acid, and 3-amino-8-cyclohexyl-6-methyl-octanoic acid; WO 2004/092132 and US 2005/0143427, including [2(S),3a(S),7a(S)]-octahydroindole-2-carboxylic acid, and [1aS,1bS,5aS,6a5]-octahydro-6-aza-cyclopropa[α]indene-6a-carboxylic acid; WO 06/114707, including 2,5,5-trimethyl-L-norleucine, (2S)-2-amino-3-cyclopentyl-2-methyl-propanoic acid, (2S)-2-amino-3-cyclobutyl-2-methyl-propanoic acid, (2S)-2-amino-4-ethyl-2-methyl-hexanoic acid, (2S)-2-amino-5-ethyl-2-methyl-heptanoic acid, and (2S,5R)-2-amino-2,5-dimethyl-heptanoic acid; U.S. Pat. No. 7,053,122, including 2-aminomethyl-5-chloro-benzoic acid, 2-aminomethyl-4,5-dichloro-benzoic acid, 2-aminomethyl-3-bromo-benzoic acid, 2-aminomethyl-6-chloro-benzoic acid, 2-(1-aminoethyl)-benzoic acid, and 2,3-dihydro-1H-isoindole-4-carboxylic acid; WO 06/120544, including (2S)-3-amino-2-(3-chlorobenzyl)-propanoic acid, (2S)-3-amino-2-(2,5-dichlorobenzyl)-propanoic acid, (2S)-3-amino-2-(3-trifluoromethylbenzyl)-propanoic acid, (2S)-3-amino-2-(3-isobutylbenzyl)-propanoic acid, (2S)-3-amino-2-(3,5-dichlorobenzyl)-propanoic acid, (2S)-3-amino-2-(3-methylthiobenzyl)-propanoic acid, (2S)-3-amino-2-(3-bromobenzyl)-propanoic acid, and (2S)-3-amino-2-(2-(4-fluorophenyl)phenyl)methyl)-propanoic acid; WO 07/052,134, including (2S)-2-aminomethyl-5-ethyl-heptanoic acid; WO 07/057,767, including (2S)-3-amino-2-(2,5-dichlorophenoxy)-propanoic acid, (2S)-3-amino-2-(3-chlorophenoxy)-propanoic acid, (2S)-3-amino-2-(2-chlorophenoxy)-propanoic acid, (2S)-3-amino-2-(2-methoxy-5-chlorophenoxy)-propanoic acid, and (2S)-3-amino-2-(3-propylphenoxy)-propanoic acid; WO 07/057,756, including (2S)-3-amino-2-(3-chlorobenzyl)-propanoic acid, (2S)-3-amino-2-(2,5-dichlorobenzyl)-propanoic acid, (2S)-3-amino-2-(3-trifluoromethylbenzyl)-propanoic acid, (2S)-3-amino-2-(3-isobutylbenzyl)-propanoic acid, (2S)-3-amino-2-(3,5-dichlorobenzyl)-propanoic acid, (2S)-3-amino-2-(3-methylthiobenzyl)-propanoic acid, (2S)-3-amino-2-(3-bromobenzyl)-propanoic acid, and (2S)-3-amino-2-((2-(4-fluorophenyl)phenyl)methyl)-propanoic acid; and WO 09/041,453, including (1S,5R,6R)-6-aminomethyl-bicyclo[3.2.0]hept-3-ene-6-acetic acid, (1R,5S,6S)-6-aminomethyl-3-methyl-bicyclo[3.2.0]hept-3-ene-6-acetic acid, (1R,5S,6S)-6-aminomethyl-3-ethyl-bicyclo[3.2.0]hept-3-ene-6-acetic acid, (1S,5R,6R)-6-aminomethyl-3-methyl-bicyclo[3.2.0]hept-3-ene-6-acetic acid, (1S,5R,6R)-6-aminomethyl-3-ethyl-bicyclo[3.2.0]hept-3-ene-6-acetic acid, and (1S,5R,6R)-6-aminomethyl-1-ethyl-bicyclo[3.2.0]hept-3-ene-6-acetic acid. Rapid systemic clearance and/or poor oral bioavailability are significant problems with many alpha-2-delta ligands such as gabapentin, which consequently require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1,800-3,600 mg/day in divided doses) are typically used for the treatment of neuropathic pain states. The use of sustained released formulations is a solution to the problem of rapid systemic clearance, as is well known to those of skill in the art.

Furthermore, many alpha-2-delta ligands are not absorbed via the large intestine but rather are absorbed in the small intestine by the large neutral amino acid transporter (LNAA) (Jezyk et al., Pharm. Res. 1999, 16, 519-526). The rapid passage of many sustained release dosage forms through the proximal absorptive region of the gastrointestinal tract has prevented the successful application of sustained release technologies to many alpha-2-delta ligands.

To address the problems of rapid systemic clearance and poor oral bioavailability, colonically absorbable prodrugs of certain alpha-2-delta ligands that provide enhanced pharmacokinetics have been developed and are disclosed, for example, in WO 01/90052; US 2002/0107208; WO 02/36118; US 2003/0144214; US 2004/0248811; U.S. Pat. No. 6,818,787; WO 05/0070483; U.S. Pat. No. 6,972,341; U.S. Pat. No. 7,026,351; U.S. Pat. No. 7,060,727; U.S. Pat. No. 7,186,855; U.S. Pat. No. 7,420,002; and U.S. Pat. No. 7,569,576.

### SUMMARY

There is a significant need for colonically absorbable prodrugs of other alpha-2-delta ligands and for effective sustained release formulations comprising such prodrugs of alpha-2-delta ligands.

In a first aspect, compounds of Formula (II) are provided:

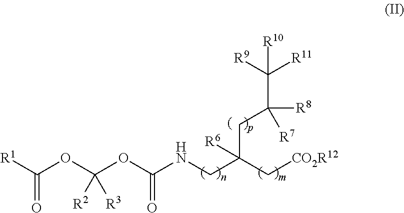

isomers thereof, and pharmaceutically acceptable salts of any of any of the foregoing, wherein:

each of m and n is 1;

p is selected from 0 and 1;

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;

R⁶ is selected from hydrogen, methyl, fluoromethyl, difluoromethyl, and trifluoromethyl;

R⁷, R⁸, and R⁹ are independently selected from hydrogen and C₁₋₆ alkyl;

R¹⁰ and R¹¹ are independently selected from hydrogen and C₁₋₆ alkyl; or R¹⁰ and R¹¹ together with the carbon atom to which they are bonded form a C₃₋₆ cycloalkyl ring; and

R² is selected from hydrogen and C₁₋₆ alkyl;

wherein the moiety having the formula:

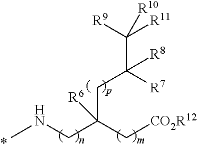

is derived from an alpha-2-delta ligand of the formula:

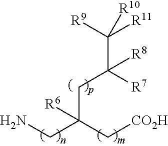

wherein the alpha-2-delta ligand is selected from:
(3S,5R)-3-(aminomethyl)-5-methylheptanoic acid;
(3R,4R,5R)-3-(aminomethyl)-4,5-dimethylheptanoic acid;
(3S,5R)-3-(aminomethyl)-5-methyloctanoic acid;
(3R,4R,5R)-3-(aminomethyl)-4,5-dimethyloctanoic acid;
(3S,5R)-3-(aminomethyl)-5-methylnonanoic acid;
(3R,4R,5R)-3-(aminomethyl)-4,5-dimethylnonanoic acid;
(3S,5R)-3-(aminomethyl)-5-methyldecanoic acid; and
(3S,5R)-3-(aminomethyl)-5-methylundecanoic acid.

In a second aspect, pharmaceutical compositions are provided comprising a therapeutically effective amount of a compound of Formula (II) or a pharmaceutically acceptable salt thereof, and a pharmaceutically acceptable vehicle.

In a third aspect, methods of treating a disease in a patient are provided comprising administering to a patient in need of such treatment a therapeutically effective amount of a compound of Formula (II).

In a fourth aspect, compounds of Formula (VII) are provided:

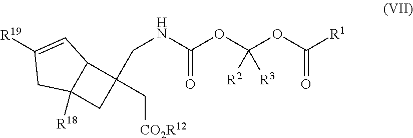

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and substituted C₃₋₈ heterocycloalkyl ring;

R¹² is selected from hydrogen and C₁₋₆ alkyl; and

R¹⁸ and R¹⁹ are independently selected from hydrogen and C₁₋₆ alkyl.

In a fifth aspect, pharmaceutical compositions are provided comprising a therapeutically effective amount of a compound of Formula (VII) or a pharmaceutically acceptable salt thereof, and a pharmaceutically acceptable vehicle.

In a sixth aspect, methods of treating a disease in a patient are provided comprising administering to a patient in need of such treatment a therapeutically effective amount of a compound of Formula (VII).

### DETAILED DESCRIPTION

### Definitions

A dash (“-”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, and alkynyl are used. In certain embodiments, an alkyl group can have from 1 to 20 carbon atoms (C₁₋₂₀) in certain embodiments, from 1 to 10 carbon atoms (C₁₋₁₀), in certain embodiments from 1 to 8 carbon atoms (C₁₋₈), in certain embodiments, from 1 to 6 carbon atoms (C₁₋₆), in certain embodiments from 1 to 4 carbon atoms (C₁₋₄, and in certain embodiments, from 1 to 3 carbon atoms (C₁₋₃).

“Acyl” by itself or as part of another substituent refers to a radical —C(O)R³⁰, where R³⁰ is chosen from hydrogen, alkyl, cycloalkyl, cycloheteroalkyl, aryl, arylalkyl, heteroalkyl, heteroaryl, and heteroarylalkyl as defined herein. Representative examples include, but are not limited to formyl, acetyl, cyclohexylcarbonyl, cyclohexylmethylcarbonyl, benzoyl, benzylcarbonyl and the like. In certain embodiments, an acyl group is C₁₋₈ acyl, C₁₋₆ acyl, and in certain embodiments, C₁₋₃ acyl.

“Alkoxy” by itself or as part of another substituent refers to a radical —OR¹¹ where R¹¹ is chosen from alkyl, heteroalkyl, cycloalkyl, heterocycloalkyl, cycloalkylalkyl, heterocycloalkylalkyl, aryl, heteroaryl, arylalkyl, and heteroarylalkyl, as defined herein. Examples of alkoxy groups include, but are not limited to, methoxy, ethoxy, propoxy, butoxy, cyclohexyloxy, and the like. In certain embodiments, an alkoxy group is C₁₋₁₈ alkoxy, in certain embodiments, C₁₋₁₂ alkoxy, in certain embodiments, C₁₋₆ alkoxy, in certain embodiments, C₁₋₄ alkoxy, and in certain embodiments, C₁₋₃ alkoxy.

“Alkoxycarbonyl” by itself or as part of another substituent refers to a radical —C(O)OR³² where R³² represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like. In certain embodiments, an alkoxycarbonyl group is selected from C₁₋₈ alkoxycarbonyl, in certain embodiments C₁₋₆ alkoxycarbonyl, and in certain embodiments C₁₋₃ alkoxycarbonyl.

“Alkylamino” by itself or as part of another substituent refers to a radical —NHR³¹ where R³¹ represents an alkyl or cycloalkyl group as defined herein. Representative examples include, but are not limited to, methylamino, ethylamino, 1-methylethylamino, cyclohexyl amino and the like.

“Alpha-2-delta ligand” refers to a molecule that binds to any subtype of the calcium channel α₂δ subunit. In certain embodiments an alpha-2-delta ligand is an α-amino acid, a β-amino acid, a γ-amino acid, or an aminoalkyl-benzoic acid.

“Aryl” by itself or as part of another substituent refers to a monovalent aromatic hydrocarbon radical derived by the removal of one hydrogen atom from a single carbon atom of a parent aromatic ring system. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms chosen from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. Aryl benzene includes bicyclic ring systems wherein at least one ring is carbocyclic and aromatic, for example, naphthalene, indane, and tetralin; and tricyclic ring systems wherein at least one ring is carbocyclic and aromatic, for example, fluorene. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀). Aryl, however, does not encompass or overlap in any way with heteroaryl, separately defined herein. In certain embodiments, aryl is phenyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀. In certain embodiments, an arylalkyl group is C₇₋₁₈ arylalkyl, e.g., the alkanyl, alkenyl or alkynyl moiety of the arylalkyl group is C₁₋₈ and the aryl moiety is C₆₋₁₀. In certain embodiments an arylalkyl group is C₇₋₉ arylalkyl, wherein the alkyl moiety is C₁₋₃ alkyl and the aryl moiety is phenyl.

“Carbamoyl” by itself or as part of another substituent refers to the radical —C(O)N(R³³)R³⁴ where R³³ and R³⁴ are independently hydrogen, alkyl, substituted alkyl, aryl, substituted aryl, arylalkyl, substituted arylalkyl, heteroarylalkyl, substituted heteroarylalkyl, heteroaryl or substituted heteroaryl, as defined herein.

“Compounds” of Formulae (I)-(VII) disclosed herein include any specific compounds within these formulae. Compounds may be identified either by their chemical structure and/or chemical name. Compounds are named using Chemistry 4-D Draw Pro, version 7.01c (ChemInnovation Software, Inc., San Diego, Calif.). When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formulae (I)-(VII) include, but are not limited to, optical isomers of compounds of Formulae (I)-(VII), racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formulae (I)-(VII) include Z- and E-forms (or cis- and trans-forms) of compounds with double bonds.

Compounds of Formulae (I)-(VII) may also exist in several tautomeric forms including the enol form, the keto form, and mixtures thereof. Accordingly, the chemical structures depicted herein encompass all possible tautomeric forms of the illustrated compounds. Compounds of Formulae (I)-(VII) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O etc. Compounds may exist in unsolvated forms as well as solvated forms, including hydrated forms and as N-oxides. In general, compounds of Formulae (I)-(VII) may be hydrated, solvated, or N-oxides. In general, compounds as referred to herein may be salts, free acid, hydrated, solvated, N-oxides or combinations of any of the foregoing. The compound may exist in multiple crystalline, co-crystalline, or amorphous forms. The compounds of Formulae (I)-(VII) include pharmaceutically acceptable salts thereof, or pharmaceutically acceptable solvates of the free acid form of any of the foregoing, as well as crystalline forms of any of the foregoing.

As referred to herein, compounds of Formulae (I)-(VII) also include solvates. A solvate refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Cycloalkyl” by itself or as part of another substituent refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl. In certain embodiments, cycloalkyl is chosen from cyclopropyl, cyclopentyl, and cyclohexyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀. In certain embodiments, a cycloalkylalkyl group is C₄₋₂₀ cycloalkylalkyl, e.g., the alkanyl, alkenyl, or alkynyl moiety of the cycloalkylalkyl group is C₁₋₈ and the cycloalkyl moiety is C₃₋₁₂. In certain embodiments, cycloalkylalkyl is C₄₋₉ cycloalkylalkyl, wherein the alkyl moiety is C₁₋₃ alkyl, and the cycloalkyl moiety is C₃₋₆ cycloalkyl.

“Cycloheteroalkyl” by itself or as part of another substituent refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature cycloheteroalkanyl or cycloheteroalkenyl is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Derived from an alpha-2-delta ligand” refers to a moiety that is structurally related to a alpha-2-delta ligand. The structure of the moiety is identical to the compound except at one or two positions. At these positions, a hydrogen atom attached to the amino group, and (optionally) the hydroxyl moiety of the carboxylic acid group has been replaced with a covalent bond that serves as a point of attachment to another moiety.

“Dialkylamino” by itself or as part of another substituent refers a radical —NR³⁵R³⁶ where R³⁵ and R³⁶ are independently an alkyl or cycloalkyl group as defined herein. Representative examples include, but are not limited to, dimethylamino, methylethylamino, di-(1-methylethyl)amino, (cyclohexyl)(methyl)amino, (cyclohexyl)(ethyl)amino, (cyclohexyl)(propyl)amino and the like.

“Disease” refers to a disease, disorder, condition, or symptom of any of the foregoing.

“Drug” as defined under 21 U.S.C. §321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . .”

“Halogen” refers to a fluoro, chloro, bromo, or iodo group. In certain embodiments, halogen refers to a chloro group.

“Heteroalkyl” by itself or as part of another substituent refers to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, NR³⁷, ═N—N═, —N═N—, —N═N—NR³⁷—, —PR³⁷—, —P(O)₂—, —POR³⁷—, —O—P(O)₂—, —SO—, —SO₂—, —Sn(R³⁷)₂—, and the like, where each R³⁷ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatoms, groups having four carbon atoms and two heteroatoms, etc. In certain embodiments, each R³⁷ is independently chosen from hydrogen and C₁₋₃ alkyl. In certain embodiments, a heteroatomic group is chosen from —O—, —S—, —NH—, —N(CH₃) —, and —SO₂—.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which may be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like. In certain embodiments, a heteroaryl group is from 4- to 20-membered heteroaryl (C₄₋₂₀), and in certain embodiments from 4- to 12-membered heteroaryl (C₄₋₁₀). In certain embodiments, heteroaryl groups are those derived from thiophene, pyrrole, benzothiophene, benzofuran, indole, pyridine, quinoline, imidazole, oxazole, or pyrazine. For example, in certain embodiments, heteroaryl is C₅ heteroaryl and is chosen from furyl, thienyl, pyrrolyl, imidazolyl, pyrazolyl, isothiazolyl, isoxazolyl. In certain embodiments, heteroaryl is C₆ heteroaryl, and is chosen from pyridinyl, pyrazinyl, pyrimidinyl, and pyridazinyl.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. In certain embodiments, a heteroarylalkyl group is C₆₋₃₀ heteroarylalkyl, e.g., the alkanyl, alkenyl, or alkynyl moiety of the heteroarylalkyl is 1- to 10-membered and the heteroaryl moiety is a 5- to 20-membered heteroaryl. In certain embodiments, a heteroarylalkyl group is C₆-20 heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, heterocycloalkyl is C₅ heterocycloalkyl and is chosen from pyrrolidinyl, tetrahydrofuranyl, tetrahydrothiophenyl, imidazolidinyl, oxazolidinyl, thiazolidinyl, doxolanyl, and dithiolanyl. In certain embodiments, heterocycloalkyl is C₆ heterocycloalkyl and is chosen from piperidinyl, tetrahydropyranyl, piperizinyl, oxazinyl, dithianyl, and dioxanyl.

“Parent aromatic ring system” refers to an unsaturated cyclic or polycyclic ring system having a conjugated π(pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt. The term “pharmaceutically acceptable salt” includes hydrates and other solvates, as well as salts in crystalline or non-crystalline form.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound, such as a compound of one of Formulae (I)-(VII) and at least one pharmaceutically acceptable vehicle, with which the compound is administered to a patient.

“Prodrug” refers to a derivative of a drug molecule that requires a transformation within the body to release the active drug. Prodrugs are frequently, although not necessarily, pharmacologically inactive until converted to the parent drug. Prodrugs may be obtained by bonding a promoiety (defined herein) typically via a functional group, to a drug. For example, referring to compounds of Formula (I), the promoiety is bonded to the drug, an alpha-2-delta ligand, via the amine functional group of the alpha-2-delta ligand. Compounds of Formulae (I)-(VII) are prodrugs of an alpha-2-delta ligand that can be metabolized within a patient's body to release the corresponding alpha-2-delta ligand.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach, or the agent may be supplied exogenously. For example, for a prodrug of Formula (I), the drug is an alpha-2-delta ligand and the promoiety has the structure:

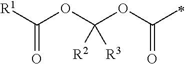

where R¹, R² and R³ are is defined herein.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecule is water.

“Substituted” refers to a group in which one or more hydrogen atoms are independently replaced with the same or different substituent(s). In certain embodiments, each substituent group is independently chosen from halogen, —OH, —CN, —CF₃, ═O, —NO₂, C₁₋₃ alkoxy, C₁₋₃ alkyl, —COOR³⁸ wherein R³⁸ is chosen from hydrogen and C₁₋₃ alkyl, and —NR³⁸₂ wherein each R³⁸ is independently chosen from hydrogen and C₁₋₃ alkyl. In certain embodiments, each substituent is independently chosen from halogen, —OH, —CN, —CF₃, —OCF₃, ═O, —NO₂, C₁₋₆ alkoxy, C₁₋₆ alkyl, —COOR³⁹, —NR³⁹₂, and —CONR³⁹₂; wherein each R³⁹ is independently chosen from hydrogen and C₁₋₆ alkyl. In certain embodiments, each substituent is independently chosen from halogen, —NH₂, —OH, C₁₋₃ alkoxy, and C₁₋₃ alkyl.

“Therapeutically effective amount” refers to the amount of a compound that, when administered to a subject for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Thioalkyl” by itself or as part of another substituent refers to a radical —SR⁴⁰ where R⁴⁰ represents an alkyl group as defined herein. In certain embodiments, a thioalkyl group is C₁₋₈ thioalkyl, in certain embodiments, C₁₋₆ thioalkyl, and in certain embodiments, C₁₋₃ thioalkyl.

“Treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treating” or “treatment” also refers to inhibiting the disease, either physically (e.g., stabilization of a discernible symptom), physiologically (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease.

Reference is now made in detail to certain embodiments of compounds, compositions, and methods. The disclosed embodiments are not intended to be limiting of the claims. To the contrary, the claims are intended to cover all alternatives, modifications, and equivalents.

### Compounds

Certain embodiments provide a compound of Formula (I):

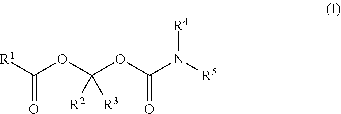

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₆₋₁₈ heteroarylalkyl, and substituted C₆₋₁₈ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring; and

—NR⁴R⁵ is a moiety derived by replacement of the hydrogen atom in an alpha-2-delta ligand of formula H—NR⁴R⁵ with a covalent bond, wherein the alpha-2-delta ligand of the formula H—NR⁴R⁵ is selected from:
(3S,5R)-3-(aminomethyl)-5-methylheptanoic acid;
(3R,4R,5R)-3-(aminomethyl)-4,5-dimethylheptanoic acid;
(3S,5R)-3-(aminomethyl)-5-methyloctanoic acid;
(3R,4R,5R)-3-(aminomethyl)-4,5-dimethyloctanoic acid;
(3S,5R)-3-(aminomethyl)-5-methylnonanoic acid;
(3R,4R,5R)-3-(aminomethyl)-4,5-dimethylnonanoic acid;
(3S,5R)-3-(aminomethyl)-5-methyldecanoic acid;
(3S,5R)-3-(aminomethyl)-5-methylundecanoic acid;
(3S,5R)-3-amino-5-methylheptanoic acid;
(3R,4R,5R)-3-amino-4,5-dimethylheptanoic acid;
(3S,5R)-3-amino-3,5-dimethylheptanoic acid;
(3R,5S)-3-amino-3,5-dimethylheptanoic acid;
3-amino-5-methyloctanoic acid;
(3S,5R)-3-amino-5-methyloctanoic acid;
(3S,4R,5R)-3-amino-4,5-dimethyloctanoic acid;
(3R,4R,5R)-3-amino-4,5-dimethyloctanoic acid;
(3R)-3-amino-3,5-dimethyloctanoic acid;
(3S,6R)-3-amino-8-cyclohexyl-6-methyloctanoic acid;
(3S,5R)-3-amino-5-methylnonanoic acid;
(4R)-2-(aminomethyl)-4-ethylheptanoic acid;
(2S)-2-(aminomethyl)-5-ethylheptanoic acid;
(2S,4S)-2-(aminomethyl)-4-ethyl-6-methylheptanoic acid;
(2R,4S)-2-(aminomethyl)-4-ethyl-6-methylheptanoic acid;
(2R,4R)-2-(aminomethyl)-4-ethyl-6-methylheptanoic acid;
(2S,4R)-2-(aminomethyl)-4-ethyl-6-methylheptanoic acid;
(2R,4S)-2-(aminomethyl)-4,7-dimethyloctanoic acid;
(2S,4S)-2-(aminomethyl)-4-ethyl-8-methylnonanoic acid;
(2S)-3-amino-2-(3-chlorobenzyl)-propanoic acid ((2S)-3-amino-2-[(3-chlorophenyl)methyl]propanoic acid);
(2S)-3-amino-2-(2,5-dichlorobenzyl)-propanoic acid ((2S)-3-amino-2-[(2,5-dichlorophenyl)methyl]propanoic acid);
(2S)-3-amino-2-(3-trifluoromethylbenzyl)-propanoic acid ((2S)-3-amino-2-[(3-trifluorophenyl)methyl]propanoic acid);
(2S)-3-amino-2-(3-isobutylbenzyl)-propanoic acid ((2S)-3-amino-2-[(3-chlorophenyl)methyl]propanoic acid);
(2S)-3-amino-2-(3,5-dichlorobenzyl)-propanoic acid ((2S)-3-amino-2-[(3,5-dichlorophenyl)methyl]propanoic acid);
(2S)-3-amino-2-(3-methylthiobenzyl)-propanoic acid ((2S)-3-amino-2-[(3-methylthiophenyl)methyl]propanoic acid);
(2S)-3-amino-2-(3-bromobenzyl)-propanoic acid ((2S)-3-amino-2-[(3-bromophenyl)methyl]propanoic acid);
(2S)-3-amino-2-((2-(4-fluorophenyl)phenyl)methyl)propanoic acid;
(2S)-3-amino-2-(2,5-dichlorophenoxy)propanoic acid;
(2S)-3-amino-2-(3-chlorophenoxy)propanoic acid;
(2S)-3-amino-2-(2-chlorophenoxy)propanoic acid;
(2S)-3-amino-2-(2-methoxy-5-chlorophenoxy)propanoic acid;
(2S)-3-amino-2-(3-propylphenoxy)propanoic acid;
(2S)-3-amino-2-(1-ethylpropylthio)propanoic acid;
(2S)-3-amino-2-(3-chlorophenylthio)propanoic acid;
(2S)-3-amino-2-(1-methylethylthio)propanoic acid;
(2S)-3-amino-2-(tert-butylthio)propanoic acid;
(2S)-3-amino-2-(2-methylpropylthio)propanoic acid;
(2S)-3-amino-2-(2-ethylbutylthio)propanoic acid;
(2S)-3-amino-2-(cyclopentylthio)propanoic acid;
(2S)-3-amino-2-(cyclohexylthio)propanoic acid;
2,5,5-trimethyl-L-norleucine (2,5,5-trimethylhexanoic acid);
(2S)-2-amino-3-cyclopentyl-2-methylpropanoic acid;
(2S)-2-amino-3-cyclobutyl-2-methylpropanoic acid;
(2S)-2-amino-4-ethyl-2-methylhexanoic acid;
(2S)-2-amino-5-ethyl-2-methylheptanoic acid;
(2S,5R)-2-amino-2,5-dimethylheptanoic acid;
(2S,4S)-4-[3-chlorophenoxy]pyrrolidine-2-carboxylic acid;
(2S,4S)-4-[(3-fluorophenyl)methyl]pyrrolidine-2-carboxylic acid;
(2S,4S)-4-[(2,3-difluorophenyl)methyl]pyrrolidine-2-carboxylic acid;
(2S,4S)-4-[(2,5-difluorophenyl)methyl]pyrrolidine-2-carboxylic acid;
(2S,4S)-4-[cyclohexylmethyl]pyrrolidine-2-carboxylic acid;
(2S,4S)-4-[(3-fluorophenoxy)methyl]pyrrolidine-2-carboxylic acid;
(2S,4S)-4-[(2,3-difluorophenoxy)methyl]pyrrolidine-2-carboxylic acid;
(2S,4S)-4-[(2,5-difluorophenoxy)methyl]pyrrolidine-2-carboxylic acid;
(2S,4S)-4-[(3,6-difluorophenoxy)methyl]pyrrolidine-2-carboxylic acid;
(2S,4S)-4-[(3-methoxyphenoxy)methyl]pyrrolidine-2-carboxylic acid;
[2(S),3a(S),7a(S)]-octahydroindole-2-carboxylic acid ([1S,6S,8S]-7-azabicyclo[4.3.0]nonane-8-carboxylic acid);
[1aS,1bS,5aS,6aS]-octahydro-6-aza-cyclopropa[α]indene-6a-carboxylic acid ((1S,6S)-5-azatricyclo[4.4.0.0<2,4.]decane-4-carboxylic acid);
(1S,5R,6R)-6-(aminomethyl)-bicyclo[3.2.0]hept-3-ene-6-acetic acid;
(1R,5S,6S)-6-(aminomethyl)-3-methyl-bicyclo[3.2.0]hept-3-ene-6-acetic acid;
(1R,5S,6S)-6-(aminomethyl)-3-ethyl-bicyclo[3.2.0]hept-3-ene-6-acetic acid;
(1S,5R,6R)-6-(aminomethyl)-3-methyl-bicyclo[3.2.0]hept-3-ene-6-acetic acid;
(1S,5R,6R)-6-(aminomethyl)-3-ethyl-bicyclo[3.2.0]hept-3-ene-6-acetic acid;
(1S,5R,6R)-6-(aminomethyl)-1-ethyl-bicyclo[3.2.0]hept-3-ene-6-acetic acid;
2-(aminomethyl)-5-chlorobenzoic acid;
2-(aminomethyl)-4,5-dichlorobenzoic acid;
2-(aminomethyl)-3-bromobenzoic acid;
2-(aminomethyl)-6-chlorobenzoic acid;
2-(1-aminoethyl)benzoic acid; and
2,3-dihydro-1H-isoindole-4-carboxylic acid.

Certain embodiments provide a compound of Formula (II):

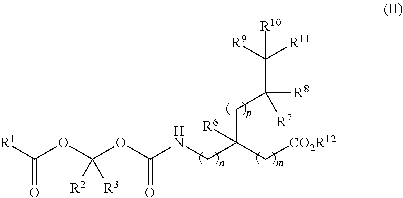

isomers thereof, and pharmaceutically acceptable salts of any of any of the foregoing, wherein:

each of m, n and p is independently selected from 0 and 1;

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;

R⁶ is selected from hydrogen, methyl, fluoromethyl, difluoromethyl, and trifluoromethyl;

R⁷, R⁸, and R⁹ are independently selected from hydrogen and C₁₋₆ alkyl;

R¹⁰ and R¹¹ are independently selected from hydrogen and C₁₋₆ alkyl; or R¹⁰ and R¹¹ together with the carbon atom to which they are bonded form a C₃₋₆ cycloalkyl ring; and

R¹² is selected from hydrogen and C₁₋₆ alkyl.

Certain embodiments provide a compound of Formula (II):

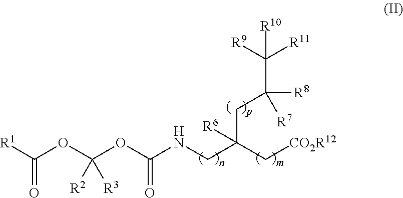

isomers thereof, and pharmaceutically acceptable salts of any of any of the foregoing, wherein:

each of m and n is 1;

p is selected from 0 and 1;

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;

R⁶ is selected from hydrogen, methyl, fluoromethyl, difluoromethyl, and trifluoromethyl;

R⁷, R⁸, and R⁹ are independently selected from hydrogen and C₁₋₆ alkyl;

R¹⁰ and R¹¹ are independently selected from hydrogen and C₁₋₆ alkyl; or R¹⁰ and R¹¹ together with the carbon atom to which they are bonded form a C₃₋₆ cycloalkyl ring; and

R¹² is selected from hydrogen and C₁₋₆ alkyl.

In certain embodiments of a compound of Formula (II), m, n, and p are independently selected from 0 and 1; R⁶ is selected from hydrogen and methyl; R⁷ is selected from hydrogen, methyl, ethyl, cyclopentyl, and cyclobutyl; R⁸ is hydrogen; R⁹ is selected from hydrogen and methyl; R¹⁰ is selected from C₁₋₄ alkyl, and R¹¹ is hydrogen, or R¹⁰ and R¹¹ together with the carbon atom to which they are bonded from a cyclohexyl ring; and R¹² is hydrogen.

In certain embodiments of a compound of Formula (II), both m and n are 0; m is 0 and n is 1; and in certain embodiments, m is 1 and n is 0. In certain embodiments of a compound of Formula (II), both m and n are not 1. In certain embodiments of a compound of Formula (II), both m and n are 1. In certain embodiments of a compound of Formula (II) the moiety:

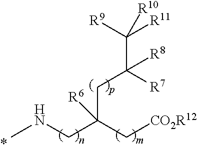

is derived from an alpha-2-delta ligand of the formula:

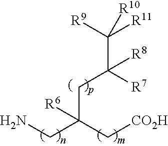

by replacing a hydrogen of the amine group with a covalent bond and by replacing the hydrogen atom of the carboxylic acid group with —R¹². In certain embodiments of a compound of Formula (II), in the alpha-2-delta ligand of the above formula, m, n, and p are independently selected from 0 and 1; R⁶ is selected from hydrogen and methyl; R⁷ is selected from hydrogen, methyl, ethyl, cyclopentyl, and cyclobutyl; R⁸ is hydrogen; R⁹ is selected from hydrogen and methyl; R¹⁰ is selected from C₁₋₄ alkyl, and R¹¹ is hydrogen, or R¹⁰ and R¹¹ together with the carbon atom to which they are bonded from a cyclohexyl ring; and R¹² is hydrogen. In certain embodiments of a compound of Formula (II), in the alpha-2-delta ligand of the above formula, both m and n are 0; m is 0 and n is 1; and in certain embodiments, m is 1 and n is 0. In certain embodiments of a compound of Formula (II), in the alpha-2-delta ligand of the above formula, both m and n are not 1. In certain embodiments of a compound of Formula (II), in the alpha-2-delta ligand of the above formula, both m and n are 1.

In certain embodiments of a compound of Formula (II), the alpha-2-delta ligand is selected from:
(3S,5R)-3-(aminomethyl)-5-methylheptanoic acid;
(3R,4R,5R)-3-(aminomethyl)-4,5-dimethylheptanoic acid;
(3S,5R)-3-(aminomethyl)-5-methyloctanoic acid;
(3R,4R,5R)-3-(aminomethyl)-4,5-dimethyloctanoic acid;
(3S,5R)-3-(aminomethyl)-5-methylnonanoic acid;
(3R,4R,5R)-3-(aminomethyl)-4,5-dimethylnonanoic acid;
(3S,5R)-3-(aminomethyl)-5-methyldecanoic acid; and
(3S,5R)-3-(aminomethyl)-5-methylundecanoic acid.

In certain embodiments of a compound of Formula (II), the compound is selected from:
3-{[((1R)-1-acetyloxyethoxy)carbonylamino]methyl}(3S,5R)-5-methylheptanoic acid;
3-{[((1S)-1-acetyloxyethoxy)carbonylamino]methyl}(3S,5R)-5-methylheptanoic acid;
3-{[((1R)-1-propanoyloxyethoxy)carbonylamino]methyl}(3S,5R)-5-methylheptanoic acid;
3-{[((1S)-1-propanoyloxyethoxy)carbonylamino]methyl}(3S,5R)-5-methylheptanoic acid;
3-({[(1R)-1-(2-methylpropanoyloxy)ethoxy]carbonylamino}methyl)(3S,5R)-5-methylheptanoic acid;
3-({[(1S)-1-(2-methylpropanoyloxy)ethoxy]carbonylamino}methyl)(3S,5R)-5-methylheptanoic acid;
3-{[((1R)-1-acetyloxy-2-methylpropoxy)carbonylamino]methyl}(3S,5R)-5-methylheptanoic acid;
3-{[((1S)-1-acetyloxy-2-methylpropoxy)carbonylamino]methyl}(3S,5R)-5-methylheptanoic acid;
3-{[((1R)-2-methyl-1-propanoyloxypropoxy)carbonylamino]methyl}(3S,5R)-5-methylheptanoic acid;
3-{[((1S)-2-methyl-1-propanoyloxypropoxy)carbonylamino]methyl}(3S,5R)-5-methylheptanoic acid;
3-({[(1R)-2-methyl-1-(2-methylpropanoyloxy)propoxy]carbonylamino}methyl)(3S,5R)-5-methylheptanoic acid;
3-({[(1S)-2-methyl-1-(2-methylpropanoyloxy)propoxy]carbonylamino}methyl)(3S,5R)-5-methylheptanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3R,4R,5R)-4,5-dimethylheptanoic acid;
3-{[((1S)-1-acetyloxyethoxy)carbonylamino]methyl}(3R,4R,5R)-4,5-dimethylheptanoic acid;
3-{[((1R)-1-propanoyloxyethoxy)carbonylamino]methyl}(3R,4R,5R)-4,5-dimethylheptanoic acid;
3-{[((1S)-1-propanoyloxyethoxy)carbonylamino]methyl}(3R,4R,5R)-4,5-dimethylheptanoic acid;
3-({[(1R)-1-(2-methylpropanoyloxy)ethoxy]carbonylamino}methyl)(3R,4R,5R)-4,5-dimethylheptanoic acid;
3-({[(1S)-1-(2-methylpropanoyloxy)ethoxy]carbonylamino}methyl)(3R,4R,5R)-4,5-dimethylheptanoic acid;
3-{[((1R)-1-acetyloxy-2-methylpropoxy)carbonylamino]methyl}(3R,4R,5R)-4,5-dimethylheptanoic acid;
3-{[((1S)-1-acetyloxy-2-methylpropoxy)carbonylamino]methyl}(3R,4R,5R)-4,5-dimethylheptanoic acid;
3-{[((1R)-2-methyl-1-propanoyloxypropoxy)carbonylamino]methyl}(3R,4R,5R)-4,5-dimethylheptanoic acid;
3-{[((1S)-2-methyl-1-propanoyloxypropoxy)carbonylamino]methyl}(3R,4R,5R)-4,5-dimethylheptanoic acid;
3-({[(1R)-2-methyl-1-(2-methylpropanoyloxy)propoxy]carbonylamino}methyl)(3R,4R,5R)-4,5-dimethylheptanoic acid;
3-({[(1S)-2-methyl-1-(2-methylpropanoyloxy)propoxy]carbonylamino}methyl)(3R,4R,5R)-4,5-dimethylheptanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3S,5R)-5-methyloctanoic acid;
3-{[((1S)-1-acetyloxyethoxy)carbonylamino]methyl}(3S,5R)-5-methyloctanoic acid;
3-{[((1R)-1-propanoyloxyethoxy)carbonylamino]methyl}(3S,5R)-5-methyloctanoic acid;
3-{[((1S)-1-propanoyloxyethoxy)carbonylamino]methyl}(3S,5R)-5-methyloctanoic acid;
3-({[(1R)-1-(2-methylpropanoyloxy)ethoxy]carbonylamino}methyl)(3S,5R)-5-methyloctanoic acid;
3-({[(1S)-1-(2-methylpropanoyloxy)ethoxy]carbonylamino}methyl)(3S,5R)-5-methyloctanoic acid;
3-{[((1R)-1-acetyloxy-2-methylpropoxy)carbonylamino]methyl}(3S,5R)-5-methyloctanoic acid;
3-{[((1S)-1-acetyloxy-2-methylpropoxy)carbonylamino]methyl}(3S,5R)-5-methyloctanoic acid;
3-{[((1R)-2-methyl-1-propanoyloxypropoxy)carbonylamino]methyl}(3S,5R)-5-methyloctanoic acid;
3-{[((1S)-2-methyl-1-propanoyloxypropoxy)carbonylamino]methyl}(3S,5R)-5-methyloctanoic acid;
3-({[(1R)-2-methyl-1-(2-methylpropanoyloxy)propoxy]carbonylamino}methyl)(3S,5R)-5-methyloctanoic acid;
3-({[(1S)-2-methyl-1-(2-methylpropanoyloxy)propoxy]carbonylamino}methyl)(3S,5R)-5-methyloctanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3R,4R,5R)-4,5-dimethyloctanoic acid;
3-{[((1S)-1-acetyloxyethoxy)carbonylamino]methyl}(3R,4R,5R)-4,5-dimethyloctanoic acid;
3-{[((1R)-1-propanoyloxyethoxy)carbonylamino]methyl}(3R,4R,5R)-4,5-dimethyloctanoic acid;
3-{[((1S)-1-propanoyloxyethoxy)carbonylamino]methyl}(3R,4R,5R)-4,5-dimethyloctanoic acid;
3-({[(1R)-1-(2-methylpropanoyloxy)ethoxy]carbonylamino}methyl)(3R,4R,5R)-4,5-dimethyloctanoic acid;
3-({[(1S)-1-(2-methylpropanoyloxy)ethoxy]carbonylamino}methyl)(3R,4R,5R)-4,5-dimethyloctanoic acid;
3-{[((1R)-1-acetyloxy-2-methylpropoxy)carbonylamino]methyl}(3R,4R,5R)-4,5-dimethyloctanoic acid;
3-{[((1S)-1-acetyloxy-2-methylpropoxy)carbonylamino]methyl}(3R,4R,5R)-4,5-dimethyloctanoic acid;
3-{[((1R)-2-methyl-1-propanoyloxypropoxy)carbonylamino]methyl}(3R,4R,5R)-4,5-dimethyloctanoic acid;
3-{[((1S)-2-methyl-1-propanoyloxypropoxy)carbonylamino]methyl}(3R,4R,5R)-4,5-dimethyloctanoic acid;
3-({[(1R)-2-methyl-1-(2-methylpropanoyloxy)propoxy]carbonylamino}methyl) (3R,4R,5R)-4,5-dimethyloctanoic acid;
3-({[(1S)-2-methyl-1-(2-methylpropanoyloxy)propoxy]carbonylamino}methyl) (3R,4R,5R)-4,5-dimethyloctanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3S,5R)-5-methylnonanoic acid;
3-{[((1S)-1-acetyloxyethoxy)carbonylamino]methyl}(3S,5R)-5-methylnonanoic acid;
3-{[((1R)-1-propanoyloxyethoxy)carbonylamino]methyl}(3S,5R)-5-methylnonanoic acid;
3-{[((1S)-1-propanoyloxyethoxy)carbonylamino]methyl}(3S,5R)-5-methylnonanoic acid;
3-({[(1R)-1-(2-methylpropanoyloxy)ethoxy]carbonylamino}methyl)(3S,5R)-5-methylnonanoic acid;
3-({[(1S)-1-(2-methylpropanoyloxy)ethoxy]carbonylamino}methyl)(3S,5R)-5-methylnonanoic acid;
3-{[((1R)-1-acetyloxy-2-methylpropoxy)carbonylamino]methyl}(3S,5R)-5-methylnonanoic acid;
3-{[((1S)-1-acetyloxy-2-methylpropoxy)carbonylamino]methyl}(3S,5R)-5-methylnonanoic acid;
3-{[((1R)-2-methyl-1-propanoyloxypropoxy)carbonylamino]methyl}(3S,5R)-5-methylnonanoic acid;
3-{[((1S)-2-methyl-1-propanoyloxypropoxy)carbonylamino]methyl}(3S,5R)-5-methylnonanoic acid;
3-({[(1R)-2-methyl-1-(2-methylpropanoyloxy)propoxy]carbonylamino}methyl)(3S,5R)-5-methylnonanoic acid;
3-({[(1S)-2-methyl-1-(2-methylpropanoyloxy)propoxy]carbonylamino}methyl)(3S,5R)-5-methylnonanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3R,4R,5R)-4,5-dimethylnonanoic acid;
3-{[((1S)-1-acetyloxyethoxy)carbonylamino]methyl}(3R,4R,5R)-4,5-dimethylnonanoic acid;
3-{[((1R)-1-propanoyloxyethoxy)carbonylamino]methyl}(3R,4R,5R)-4,5-dimethylnonanoic acid;
3-{[((1S)-1-propanoyloxyethoxy)carbonylamino]methyl}(3R,4R,5R)-4,5-dimethylnonanoic acid;
3-({[(1R)-1-(2-methylpropanoyloxy)ethoxy]carbonylamino}methyl)(3R,4R,5R)-4,5-dimethylnonanoic acid;
3-({[(1S)-1-(2-methylpropanoyloxy)ethoxy]carbonylamino}methyl)(3R,4R,5R)-4,5-dimethylnonanoic acid;
3-{[((1R)-1-acetyloxy-2-methylpropoxy)carbonylamino]methyl}(3R,4R,5R)-4,5-dimethylnonanoic acid;
3-{[((1S)-1-acetyloxy-2-methylpropoxy)carbonylamino]methyl}(3R,4R,5R)-4,5-dimethylnonanoic acid;
3-{[((1R)-2-methyl-1-propanoyloxypropoxy)carbonylamino]methyl}(3R,4R,5R)-4,5-dimethylnonanoic acid;
3-{[((1S)-2-methyl-1-propanoyloxypropoxy)carbonylamino]methyl}(3R,4R,5R)-4,5-dimethylnonanoic acid;
3-({[(1R)-2-methyl-1-(2-methylpropanoyloxy)propoxy]carbonylamino}methyl)(3R,4R,5R)-4,5-dimethylnonanoic acid;
3-({[(1S)-2-methyl-1-(2-methylpropanoyloxy)propoxy]carbonylamino}methyl)(3R,4R,5R)-4,5-dimethylnonanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3S,5R)-5-methyldecanoic acid;
3-{[((1S)-1-acetyloxyethoxy)carbonylamino]methyl}(3S,5R)-5-methyldecanoic acid;
3-{[((1R)-1-propanoyloxyethoxy)carbonylamino]methyl}(3S,5R)-5-methyldecanoic acid;
3-{[((1S)-1-propanoyloxyethoxy)carbonylamino]methyl}(3S,5R)-5-methyldecanoic acid;
3-({[(1R)-1-(2-methylpropanoyloxy)ethoxy]carbonylamino}methyl)(3S,5R)-5-methyldecanoic acid;
3-({[(1S)-1-(2-methylpropanoyloxy)ethoxy]carbonylamino}methyl)(3S,5R)-5-methyldecanoic acid;
3-{[((1R)-1-acetyloxy-2-methylpropoxy)carbonylamino]methyl}(3S,5R)-5-methyldecanoic acid;
3-{[((1S)-1-acetyloxy-2-methylpropoxy)carbonylamino]methyl}(3S,5R)-5-methyldecanoic acid;
3-{[((1R)-2-methyl-1-propanoyloxypropoxy)carbonylamino]methyl}(3S,5R)-5-methyldecanoic acid;
3-{[((1S)-2-methyl-1-propanoyloxypropoxy)carbonylamino]methyl}(3S,5R)-5-methyldecanoic acid;
3-({[(1R)-2-methyl-1-(2-methylpropanoyloxy)propoxy]carbonylamino}methyl)(3S,5R)-5-methyldecanoic acid;
3-({[(1S)-2-methyl-1-(2-methylpropanoyloxy)propoxy]carbonylamino}methyl)(3S,5R)-5-methyldecanoic acid; and

a pharmaceutically acceptable salt of any of the foregoing.

In certain embodiments of a compound of Formula (II), the compound is selected from:
3-{[((1R)-1-acetyloxyethoxy)carbonylamino]methyl}(3S,5R)-5-methylundecanoic acid;
3-{[((1S)-1-acetyloxyethoxy)carbonylamino]methyl}(3S,5R)-5-methylundecanoic acid;
3-{[((1R)-1-propanoyloxyethoxy)carbonylamino]methyl}(3S,5R)-5-methylundecanoic acid;
3-{[((1S)-1-propanoyloxyethoxy)carbonylamino]methyl}(3S,5R)-5-methylundecanoic acid;
3-({[(1R)-1-(2-methylpropanoyloxy)ethoxy]carbonylamino}methyl)(3S,5R)-5-methylundecanoic acid;
3-({[(1S)-1-(2-methylpropanoyloxy)ethoxy]carbonylamino}methyl)(3S,5R)-5-methylundecanoic acid;
3-{[((1R)-1-acetyloxy-2-methylpropoxy)carbonylamino]methyl}(3S,5R)-5-methylundecanoic acid;
3-{[((1S)-1-acetyloxy-2-methylpropoxy)carbonylamino]methyl}(3S,5R)-5-methylundecanoic acid;
3-{[((1R)-2-methyl-1-propanoyloxypropoxy)carbonylamino]methyl}(3S,5R)-5-methylundecanoic acid;
3-{[((1S)-2-methyl-1-propanoyloxypropoxy)carbonylamino]methyl}(3S,5R)-5-methylundecanoic acid;
3-({[(1R)-2-methyl-1-(2-methylpropanoyloxy)propoxy]carbonylamino}methyl)(3S,5R)-5-methylundecanoic acid;
3-({[(1S)-2-methyl-1-(2-methylpropanoyloxy)propoxy]carbonylamino}methyl)(3S,5R)-5-methylundecanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,5R)-5-methylheptanoic acid;
3-[((1S)-1-acetyloxyethoxy)carbonylamino](3S,5R)-5-methylheptanoic acid;
3-[((1R)-1-propanoyloxyethoxy)carbonylamino](3S,5R)-5-methylheptanoic acid;
3-[((1S)-1-propanoyloxyethoxy)carbonylamino](3S,5R)-5-methylheptanoic acid;
3-{[(1R)-1-(2-methylpropanoyloxy)ethoxy]carbonylamino}(3S,5R)-5-methylheptanoic acid;
3-{[(1S)-1-(2-methylpropanoyloxy)ethoxy]carbonylamino}(3S,5R)-5-methylheptanoic acid;
3-[((1R)-1-acetyloxy-2-methylpropoxy)carbonylamino](3S,5R)-5-methylheptanoic acid;
3-[((1S)-1-acetyloxy-2-methylpropoxy)carbonylamino] (3S,5R)-5-methylheptanoic acid;
3-[((1R)-2-methyl-1-propanoyloxypropoxy)carbonylamino](3S,5R)-5-methylheptanoic acid;
3-[((1S)-2-methyl-1-propanoyloxypropoxy)carbonylamino](3S,5R)-5-methylheptanoic acid;
3-{[(1R)-2-methyl-1-(2-methylpropanoyloxy)propoxy]carbonylamino}(3S,5R)-5-methylheptanoic acid;
3-{[(1S)-2-methyl-1-(2-methylpropanoyloxy)propoxy]carbonylamino}(3S,5R)-5-methylheptanoic acid; and

a pharmaceutically acceptable salt of any of the foregoing.

In certain embodiments of a compound of Formula (II), the compound is selected from:
3-[((1R)-1-acetyloxyethoxy)carbonylamino](3R,4R,5R)-4,5-dimethylheptanoic acid;
3-[((1S)-1-acetyloxyethoxy)carbonylamino] (3R,4R,5R)-4,5-dimethylheptanoic acid;
3-[((1R)-1-propanoyloxyethoxy)carbonylamino](3R,4R,5R)-4,5-dimethylheptanoic acid;
3-[((1S)-1-propanoyloxyethoxy)carbonylamino](3R,4R,5R)-4,5-dimethylheptanoic acid;
3-{[(1R)-1-(2-methylpropanoyloxy)ethoxy]carbonylamino}(3R,4R,5R)-4,5-dimethylheptanoic acid;
3-{[(1S)-1-(2-methylpropanoyloxy)ethoxy]carbonylamino}(3R,4R,5R)-4,5-dimethylheptanoic acid;
3-[((1R)-1-acetyloxy-2-methylpropoxy)carbonylamino](3R,4R,5R)-4,5-dimethylheptanoic acid;
3-[((1S)-1-acetyloxy-2-methylpropoxy)carbonylamino](3R,4R,5R)-4,5-dimethylheptanoic acid;
3-[((1R)-2-methyl-1-propanoyloxypropoxy)carbonylamino](3R,4R,5R)-4,5-dimethylheptanoic acid;
3-[((1S)-2-methyl-1-propanoyloxypropoxy)carbonylamino](3R,4R,5R)-4,5-dimethylheptanoic acid;
3-{[(1R)-2-methyl-1-(2-methylpropanoyloxy)propoxy]carbonylamino}(3R,4R,5R)-4,5-dimethylheptanoic acid;
3-{[(1S)-2-methyl-1-(2-methylpropanoyloxy)propoxy]carbonylamino}(3R,4R,5R)-4,5-dimethylheptanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,5R)-3,5-dimethylheptanoic acid;
3-[((1S)-1-acetyloxyethoxy)carbonylamino](3S,5R)-3,5-dimethylheptanoic acid;
3-[((1R)-1-propanoyloxyethoxy)carbonylamino](3S,5R)-3,5-dimethylheptanoic acid;
3-[((1S)-1-propanoyloxyethoxy)carbonylamino](3S,5R)-3,5-dimethylheptanoic acid;
3-{[(1R)-1-(2-methylpropanoyloxy)ethoxy]carbonylamino}(3S,5R)-3,5-dimethylheptanoic acid;
3-{[(1S)-1-(2-methylpropanoyloxy)ethoxy]carbonylamino}(3S,5R)-3,5-dimethylheptanoic acid;
3-[((1R)-1-acetyloxy-2-methylpropoxy)carbonylamino](3S,5R)-3,5-dimethylheptanoic acid;
3-[((1S)-1-acetyloxy-2-methylpropoxy)carbonylamino] (3S,5R)-3,5-dimethylheptanoic acid;
3-[((1R)-2-methyl-1-propanoyloxypropoxy)carbonylamino](3S,5R)-3,5-dimethylheptanoic acid;
3-[((1S)-2-methyl-1-propanoyloxypropoxy)carbonylamino](3S,5R)-3,5-dimethylheptanoic acid;
3-{[(1R)-2-methyl-1-(2-methylpropanoyloxy)propoxy]carbonylamino}(3S,5R)-3,5-dimethylheptanoic acid;
3-{[(1S)-2-methyl-1-(2-methylpropanoyloxy)propoxy]carbonylamino}(3S,5R)-3,5-dimethylheptanoic acid; and

a pharmaceutically acceptable salt of any of the foregoing.

In certain embodiments of a compound of Formula (II), the compound is selected from:
3-[((1R)-1-acetyloxyethoxy)carbonylamino] (3R,5S)-3,5-dimethylheptanoic acid;
3-[((1S)-1-acetyloxyethoxy)carbonylamino] (3R,5S)-3,5-dimethylheptanoic acid;
3-[((1R)-1-propanoyloxyethoxy)carbonylamino] (3R,5S)-3,5-dimethylheptanoic acid;
3-[((1S)-1-propanoyloxyethoxy)carbonylamino](3R,5S)-3,5-dimethylheptanoic acid;
3-{[(1R)-1-(2-methylpropanoyloxy)ethoxy]carbonylamino}(3R,5S)-3,5-dimethylheptanoic acid;
3-{[(1S)-1-(2-methylpropanoyloxy)ethoxy]carbonylamino}(3R,5S)-3,5-dimethylheptanoic acid;
3-[((1R)-1-acetyloxy-2-methylpropoxy)carbonylamino] (3R,5S)-3,5-dimethylheptanoic acid;
3-[((1S)-1-acetyloxy-2-methylpropoxy)carbonylamino] (3R,5S)-3,5-dimethylheptanoic acid;
3-[((1R)-2-methyl-1-propanoyloxypropoxy)carbonylamino] (3R,5S)-3,5-dimethylheptanoic acid;
3-[((1S)-2-methyl-1-propanoyloxypropoxy)carbonylamino] (3R,5S)-3,5-dimethylheptanoic acid;
3-{[(1R)-2-methyl-1-(2-methylpropanoyloxy)propoxy]carbonylamino}(3R,5S)-3,5-dimethylheptanoic acid;
3-{[(1S)-2-methyl-1-(2-methylpropanoyloxy)propoxy]carbonylamino}(3R,5S)-3,5-dimethylheptanoic acid; and

a pharmaceutically acceptable salt of any of the foregoing.

In certain embodiments of a compound of Formula (II), the compound is selected from:
3-[((1R)-1-acetyloxyethoxy)carbonylamino]-5-methyloctanoic acid;
3-[((1S)-1-acetyloxyethoxy)carbonylamino]-5-methyloctanoic acid;
3-[((1R)-1-propanoyloxyethoxy)carbonylamino]-5-methyloctanoic acid;
3-[((1S)-1-propanoyloxyethoxy)carbonylamino]-5-methyloctanoic acid;
3-{[(1R)-1-(2-methylpropanoyloxy)ethoxy]carbonylamino}-5-methyloctanoic acid;
3-{[(1S)-1-(2-methylpropanoyloxy)ethoxy]carbonylamino}-5-methyloctanoic acid;
3-[((1R)-1-acetyloxy-2-methylpropoxy)carbonylamino]-5-methyloctanoic acid;
3-[((1S)-1-acetyloxy-2-methylpropoxy)carbonylamino]-5-methyloctanoic acid;
3-[((1R)-2-methyl-1-propanoyloxypropoxy)carbonylamino]-5-methyloctanoic acid;
3-[((1S)-2-methyl-1-propanoyloxypropoxy)carbonylamino]-5-methyloctanoic acid;
3-{[(1R)-2-methyl-1-(2-methylpropanoyloxy)propoxy]carbonylamino}-5-methyloctanoic acid;
3-{[(1S)-2-methyl-1-(2-methylpropanoyloxy)propoxy]carbonylamino}-5-methyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,5R)-5-methyloctanoic acid;
3-[((1S)-1-acetyloxyethoxy)carbonylamino](3S,5R)-5-methyloctanoic acid;
3-[((1R)-1-propanoyloxyethoxy)carbonylamino](3S,5R)-5-methyloctanoic acid;
3-[((1S)-1-propanoyloxyethoxy)carbonylamino](3S,5R)-5-methyloctanoic acid;
3-{[(1R)-1-(2-methylpropanoyloxy)ethoxy]carbonylamino}(3S,5R)-5-methyloctanoic acid;
3-{[(1S)-1-(2-methylpropanoyloxy)ethoxy]carbonylamino}(3S,5R)-5-methyloctanoic acid;
3-[((1R)-1-acetyloxy-2-methylpropoxy)carbonylamino](3S,5R)-5-methyloctanoic acid;
3-[((1S)-1-acetyloxy-2-methylpropoxy)carbonylamino] (3S,5R)-5-methyloctanoic acid;
3-[((1R)-2-methyl-1-propanoyloxypropoxy)carbonylamino](3S,5R)-5-methyloctanoic acid;
3-[((1S)-2-methyl-1-propanoyloxypropoxy)carbonylamino](3S,5R)-5-methyloctanoic acid;
3-{[(1R)-2-methyl-1-(2-methylpropanoyloxy)propoxy]carbonylamino}(3S,5R)-5-methyloctanoic acid;
3-{[(1S)-2-methyl-1-(2-methylpropanoyloxy)propoxy]carbonylamino}(3S,5R)-5-methyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,4R,5R)-4,5-dimethyloctanoic acid;
3-[((1S)-1-acetyloxyethoxy)carbonylamino] (3S,4R,5R)-4,5-dimethyloctanoic acid;
3-[((1R)-1-propanoyloxyethoxy)carbonylamino] (3S,4R,5R)-4,5-dimethyloctanoic acid;
3-[((1S)-1-propanoyloxyethoxy)carbonylamino] (3S,4R,5R)-4,5-dimethyloctanoic acid;
3-{[(1R)-1-(2-methylpropanoyloxy)ethoxy]carbonylamino}(3S,4R,5R)-4,5-dimethyloctanoic acid;
3-{[(1S)-1-(2-methylpropanoyloxy)ethoxy]carbonylamino}(3S,4R,5R)-4,5-dimethyloctanoic acid;
3-[((1R)-1-acetyloxy-2-methylpropoxy)carbonylamino](3S,4R,5R)-4,5-dimethyloctanoic acid;
3-[((1S)-1-acetyloxy-2-methylpropoxy)carbonylamino](3S,4R,5R)-4,5-dimethyloctanoic acid;
3-[((1R)-2-methyl-1-propanoyloxypropoxy)carbonylamino](3S,4R,5R)-4,5-dimethyloctanoic acid;
3-[((1S)-2-methyl-1-propanoyloxypropoxy)carbonylamino](3S,4R,5R)-4,5-dimethyloctanoic acid;
3-{[(1R)-2-methyl-1-(2-methylpropanoyloxy)propoxy]carbonylamino}(3S,4R,5R)-4,5-dimethyloctanoic acid;
3-{[(1S)-2-methyl-1-(2-methylpropanoyloxy)propoxy]carbonylamino}(3S,4R,5R)-4,5-dimethyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3R,4R,5R)-4,5-dimethyloctanoic acid;
3-[((1S)-1-acetyloxyethoxy)carbonylamino] (3R,4R,5R)-4,5-dimethyloctanoic acid;
3-[((1R)-1-propanoyloxyethoxy)carbonylamino] (3R,4R,5R)-4,5-dimethyloctanoic acid;
3-[((1S)-1-propanoyloxyethoxy)carbonylamino] (3R,4R,5R)-4,5-dimethyloctanoic acid;
3-{[(1R)-1-(2-methylpropanoyloxy)ethoxy]carbonylamino}(3R,4R,5R)-4,5-dimethyloctanoic acid;
3-{[(1S)-1-(2-methylpropanoyloxy)ethoxy]carbonylamino}(3R,4R,5R)-4,5-dimethyloctanoic acid;
3-[((1R)-1-acetyloxy-2-methylpropoxy)carbonylamino](3R,4R,5R)-4,5-dimethyloctanoic acid;
3-[((1S)-1-acetyloxy-2-methylpropoxy)carbonylamino](3R,4R,5R)-4,5-dimethyloctanoic acid;
3-[((1R)-2-methyl-1-propanoyloxypropoxy)carbonylamino](3R,4R,5R)-4,5-dimethyloctanoic acid;
3-[((1S)-2-methyl-1-propanoyloxypropoxy)carbonylamino](3R,4R,5R)-4,5-dimethyloctanoic acid;
3-{[(1R)-2-methyl-1-(2-methylpropanoyloxy)propoxy]carbonylamino}(3R,4R,5R)-4,5-dimethyloctanoic acid;
3-{[(1S)-2-methyl-1-(2-methylpropanoyloxy)propoxy]carbonylamino}(3R,4R,5R)-4,5-dimethyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3R)-3,5-dimethyloctanoic acid;
3-[((1S)-1-acetyloxyethoxy)carbonylamino](3R)-3,5-dimethyloctanoic acid;
3-[((1R)-1-propanoyloxyethoxy)carbonylamino](3R)-3,5-dimethyloctanoic acid;
3-[((1S)-1-propanoyloxyethoxy)carbonylamino](3R)-3,5-dimethyloctanoic acid;
3-{[(1R)-1-(2-methylpropanoyloxy)ethoxy]carbonylamino}(3R)-3,5-dimethyloctanoic acid;
3-{[(1S)-1-(2-methylpropanoyloxy)ethoxy]carbonylamino}(3R)-3,5-dimethyloctanoic acid;
3-[((1R)-1-acetyloxy-2-methylpropoxy)carbonylamino](3R)-3,5-dimethyloctanoic acid;
3-[((1S)-1-acetyloxy-2-methylpropoxy)carbonylamino](3R)-3,5-dimethyloctanoic acid;
3-[((1R)-2-methyl-1-propanoyloxypropoxy)carbonylamino](3R)-3,5-dimethyloctanoic acid;
3-[((1S)-2-methyl-1-propanoyloxypropoxy)carbonylamino](3R)-3,5-dimethyloctanoic acid;
3-{[(1R)-2-methyl-1-(2-methylpropanoyloxy)propoxy]carbonylamino}(3R)-3,5-dimethyloctanoic acid;
3-{[(1S)-2-methyl-1-(2-methylpropanoyloxy)propoxy]carbonylamino}(3R)-3,5-dimethyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,6R)-8-cyclohexyl-6-methyloctanoic acid;
3-[((1S)-1-acetyloxyethoxy)carbonylamino](3S,6R)-8-cyclohexyl-6-methyloctanoic acid;
3-[((1R)-1-propanoyloxyethoxy)carbonylamino](3S,6R)-8-cyclohexyl-6-methyloctanoic acid;
3-[((1S)-1-propanoyloxyethoxy)carbonylamino](3S,6R)-8-cyclohexyl-6-methyloctanoic acid;
3-{[(1R)-1-(2-methylpropanoyloxy)ethoxy]carbonylamino}(3S,6R)-8-cyclohexyl-6-methyloctanoic acid;
3-{[(1S)-1-(2-methylpropanoyloxy)ethoxy]carbonylamino}(3S,6R)-8-cyclohexyl-6-methyloctanoic acid;
3-[((1R)-1-acetyloxy-2-methylpropoxy)carbonylamino](3S,6R)-8-cyclohexyl-6-methyloctanoic acid;
3-[((1S)-1-acetyloxy-2-methylpropoxy)carbonylamino](3S,6R)-8-cyclohexyl-6-methyloctanoic acid;
3-[((1R)-2-methyl-1-propanoyloxypropoxy)carbonylamino](3S,6R)-8-cyclohexyl-6-methyloctanoic acid;
3-[((1S)-2-methyl-1-propanoyloxypropoxy)carbonylamino](3S,6R)-8-cyclohexyl-6-methyloctanoic acid;
3-{[(1R)-2-methyl-1-(2-methylpropanoyloxy)propoxy]carbonylamino}(3S,6R)-8-cyclohexyl-6-methyloctanoic acid;
3-{[(1S)-2-methyl-1-(2-methylpropanoyloxy)propoxy]carbonylamino}(3S,6R)-8-cyclohexyl-6-methyloctanoic acid; and

a pharmaceutically acceptable salt of any of the foregoing.

In certain embodiments of a compound of Formula (II), the compound is selected from:
3-[((1R)-1-acetyloxyethoxy)carbonylamino](3S,5R)-5-methylnonanoic acid;
3-[((1S)-1-acetyloxyethoxy)carbonylamino](3S,5R)-5-methylnonanoic acid;
3-[((1R)-1-propanoyloxyethoxy)carbonylamino](3S,5R)-5-methylnonanoic acid;
3-[((1S)-1-propanoyloxyethoxy)carbonylamino](3S,5R)-5-methylnonanoic acid;
3-{[(1R)-1-(2-methylpropanoyloxy)ethoxy]carbonylamino}(3S,5R)-5-methylnonanoic acid;
3-{[(1S)-1-(2-methylpropanoyloxy)ethoxy]carbonylamino}(3S,5R)-5-methylnonanoic acid;
3-[((1R)-1-acetyloxy-2-methylpropoxy)carbonylamino](3S,5R)-5-methylnonanoic acid;
3-[((1S)-1-acetyloxy-2-methylpropoxy)carbonylamino](3S,5R)-5-methylnonanoic acid;
3-[((1R)-2-methyl-1-propanoyloxypropoxy)carbonylamino] (3S,5R)-5-methylnonanoic acid;
3-[((1S)-2-methyl-1-propanoyloxypropoxy)carbonylamino] (3S,5R)-5-methylnonanoic acid;
3-{[(1R)-2-methyl-1-(2-methylpropanoyloxy)propoxy]carbonylamino}(3S,5R)-5-methylnonanoic acid;
3-{[(1S)-2-methyl-1-(2-methylpropanoyloxy)propoxy]carbonylamino}(3S,5R)-5-methylnonanoic acid; and

a pharmaceutically acceptable salt of any of the foregoing.

In certain embodiments of a compound of Formula (II), the compound is selected from:
(4R)-2-{[((1R)-1-acetyloxyethoxy)carbonylamino]methyl}-4-ethylheptanoic acid;
(4R)-2-{[((1S)-1-acetyloxyethoxy)carbonylamino]methyl}-4-ethylheptanoic acid;
(4R)-2-{[((1R)-1-propanoyloxyethoxy)carbonylamino]methyl}-4-ethylheptanoic acid;
(4R)-2-{[((1S)-1-propanoyloxyethoxy)carbonylamino]methyl}-4-ethylheptanoic acid;
(4R)-2-({[(1R)-1-(2-methylpropanoyloxy)ethoxy]carbonylamino}methyl)-4-ethylheptanoic acid;
(4R)-2-({[(1S)-1-(2-methylpropanoyloxy)ethoxy]carbonylamino}methyl)-4-ethylheptanoic acid;
(4R)-2-{[((1R)-1-acetyloxy-2-methylpropoxy)carbonylamino]methyl}-4-ethylheptanoic acid;
(4R)-2-{[((1S)-1-acetyloxy-2-methylpropoxy)carbonylamino]methyl}-4-ethylheptanoic acid;
(4R)-2-{[((1R)-2-methyl-1-propanoyloxypropoxy)carbonylamino]methyl}-4-ethylheptanoic acid;
(4R)-2-{[((1S)-2-methyl-1-propanoyloxypropoxy)carbonylamino]methyl}-4-ethylheptanoic acid;
(4R)-2-({[(1R)-2-methyl-1-(2-methylpropanoyloxy)propoxy]carbonylamino}methyl)-4-ethylheptanoic acid;
(4R)-2-({[(1S)-2-methyl-1-(2-methylpropanoyloxy)propoxy]carbonylamino}methyl)-4-ethylheptanoic acid; and

a pharmaceutically acceptable salt of any of the foregoing.

In certain embodiments of a compound of Formula (II), the compound is selected from:
(2S)-2-{[((1R)-1-acetyloxyethoxy)carbonylamino]methyl}-5-ethylheptanoic acid;
(2S)-2-{[((1S)-1-acetyloxyethoxy)carbonylamino]methyl}-5-ethylheptanoic acid;
(2S)-2-{[((1R)-1-propanoyloxyethoxy)carbonylamino]methyl}-5-ethylheptanoic acid;
(2S)-2-{[((1S)-1-propanoyloxyethoxy)carbonylamino]methyl}-5-ethylheptanoic acid;
(2S)-2-({[(1R)-1-(2-methylpropanoyloxy)ethoxy]carbonylamino}methyl)-5-ethylheptanoic acid;
(2S)-2-({[(1S)-1-(2-methylpropanoyloxy)ethoxy]carbonylamino}methyl)-5-ethylheptanoic acid;
(2S)-2-{[((1R)-1-acetyloxy-2-methylpropoxy)carbonylamino]methyl}-5-ethylheptanoic acid;
(2S)-2-{[((1S)-1-acetyloxy-2-methylpropoxy)carbonylamino]methyl}-5-ethylheptanoic acid;
(2S)-2-{[((1R)-2-methyl-1-propanoyloxypropoxy)carbonylamino]methyl}-5-ethylheptanoic acid;
(2S)-2-{[((1S)-2-methyl-1-propanoyloxypropoxy)carbonylamino]methyl}-5-ethylheptanoic acid;
(2S)-2-({[(1R)-2-methyl-1-(2-methylpropanoyloxy)propoxy]carbonylamino}methyl)-5-ethylheptanoic acid;
(2S)-2-({[(1S)-2-methyl-1-(2-methylpropanoyloxy)propoxy]carbonylamino}methyl)-5-ethylheptanoic acid; and

a pharmaceutically acceptable salt of any of the foregoing.

In certain embodiments of a compound of Formula (II), the compound is selected from:
2-{[((1R)-1-acetyloxyethoxy)carbonylamino]methyl}(2S,4S)-4-ethyl-6-methylheptanoic acid;
2-{[((1S)-1-acetyloxyethoxy)carbonylamino]methyl}(2S,4S)-4-ethyl-6-methylheptanoic acid;
2-{[((1R)-1-propanoyloxyethoxy)carbonylamino]methyl}(2S,4S)-4-ethyl-6-methylheptanoic acid;
2-{[((1S)-1-propanoyloxyethoxy)carbonylamino]methyl}(2S,4S)-4-ethyl-6-methylheptanoic acid;
2-({[(1R)-1-(2-methylpropanoyloxy)ethoxy]carbonylamino}methyl)(2S,4S)-4-ethyl-6-methylheptanoic acid;
2-({[(1S)-1-(2-methylpropanoyloxy)ethoxy]carbonylamino}methyl)(2S,4S)-4-ethyl-6-methylheptanoic acid;
2-{[((1R)-1-acetyloxy-2-methylpropoxy)carbonylamino]methyl}(2S,4S)-4-ethyl-6-methylheptanoic acid;
2-{[((1S)-1-acetyloxy-2-methylpropoxy)carbonylamino]methyl}(2S,4S)-4-ethyl-6-methylheptanoic acid;
2-{[((1R)-2-methyl-1-propanoyloxypropoxy)carbonylamino]methyl}(2S,4S)-4-ethyl-6-methylheptanoic acid;
2-{[((1S)-2-methyl-1-propanoyloxypropoxy)carbonylamino]methyl}(2S,4S)-4-ethyl-6-methylheptanoic acid;
2-({[(1R)-2-methyl-1-(2-methylpropanoyloxy)propoxy]carbonylamino}methyl)(2S,4S)-4-ethyl-6-methylheptanoic acid;
2-({[(1S)-2-methyl-1-(2-methylpropanoyloxy)propoxy]carbonylamino}methyl)(2S,4S)-4-ethyl-6-methylheptanoic acid; and

a pharmaceutically acceptable salt of any of the foregoing.

In certain embodiments of a compound of Formula (II), the compound is selected from:
2-[((1R)-1-acetyloxyethoxy)carbonylamino](2S)-2,5,5-trimethylhexanoic acid;
2-[((1S)-1-acetyloxyethoxy)carbonylamino](2S)-2,5,5-trimethylhexanoic acid;
2-[((1R)-1-propanoyloxyethoxy)carbonylamino](2S)-2,5,5-trimethylhexanoic acid;
2-[((1S)-1-propanoyloxyethoxy)carbonylamino](2S)-2,5,5-trimethylhexanoic acid;
2-{[(1R)-1-(2-methylpropanoyloxy)ethoxy]carbonylamino}(2S)-2,5,5-trimethylhexanoic acid;
2-{[(1S)-1-(2-methylpropanoyloxy)ethoxy]carbonylamino}(2S)-2,5,5-trimethylhexanoic acid;
2-[((1R)-1-acetyloxy-2-methylpropoxy)carbonylamino](2S)-2,5,5-trimethylhexanoic acid;
2-[((1S)-1-acetyloxy-2-methylpropoxy)carbonylamino](2S)-2,5,5-trimethylhexanoic acid;
2-[((1R)-2-methyl-1-propanoyloxypropoxy)carbonylamino](2S)-2,5,5-trimethylhexanoic acid;
2-[((1S)-2-methyl-1-propanoyloxypropoxy)carbonylamino](2S)-2,5,5-trimethylhexanoic acid;
2-{[(1R)-2-methyl-1-(2-methylpropanoyloxy)propoxy]carbonylamino}(2S)-2,5,5-trimethylhexanoic acid;
2-{[(1S)-2-methyl-1-(2-methylpropanoyloxy)propoxy]carbonylamino}(2S)-2,5,5-trimethylhexanoic acid; and

a pharmaceutically acceptable salt of any of the foregoing.

In certain embodiments of a compound of Formula (II), the compound is selected from:
(2S)-2-[((1R)-1-acetyloxyethoxy)carbonylamino]-3-cyclopentyl-2-methylpropanoic acid;
(2S)-2-[((1S)-1-acetyloxyethoxy)carbonylamino]-3-cyclopentyl-2-methylpropanoic acid;
(2S)-2-[((1R)-1-propanoyloxyethoxy)carbonylamino]-3-cyclopentyl-2-methylpropanoic acid;
(2S)-2-[((1S)-1-propanoyloxyethoxy)carbonylamino]-3-cyclopentyl-2-methylpropanoic acid;
(2S)-2-{[(1R)-1-(2-methylpropanoyloxy)ethoxy]carbonylamino}-3-cyclopentyl-2-methylpropanoic acid;
(2S)-2-{[(1S)-1-(2-methylpropanoyloxy)ethoxy]carbonylamino}-3-cyclopentyl-2-methylpropanoic acid;
2-[((1R)-1-acetyloxy-2-methoxypropoxy)carbonylamino]-(2S)-3-cyclopentyl-2-methylpropanoic acid;
2-[((1S)-1-acetyloxy-2-methoxypropoxy)carbonylamino]-(2S)-3-cyclopentyl-2-methylpropanoic acid;
2-[((1R)-2-methyl-1-propanoyloxypropoxy)carbonylamino]-(2S)-3-cyclopentyl-2-methylpropanoic acid;
2-[((1S)-2-methyl-1-propanoyloxypropoxy)carbonylamino]-(2S)-3-cyclopentyl-2-methylpropanoic acid;
2-{[(1R)-2-methyl-1-(2-methylpropanoyloxy)propoxy]carbonylamino}-(2S)-3-cyclopentyl-2-methylpropanoic acid;
2-{[(1S)-2-methyl-1-(2-methylpropanoyloxy)propoxy]carbonylamino}-(2S)-3-cyclopentyl-2-methylpropanoic acid; and

a pharmaceutically acceptable salt of any of the foregoing.

In certain embodiments of a compound of Formula (II), the compound is selected from:
(2S)-2-[((1R)-1-acetyloxyethoxy)carbonylamino]-3-cyclobutyl-2-methylpropanoic acid;
(2S)-2-[((1S)-1-acetyloxyethoxy)carbonylamino]-3-cyclobutyl-2-methylpropanoic acid;
(2S)-2-[((1R)-1-propanoyloxyethoxy)carbonylamino]-3-cyclobutyl-2-methylpropanoic acid;
(2S)-2-[((1S)-1-propanoyloxyethoxy)carbonylamino]-3-cyclobutyl-2-methylpropanoic acid;
(2S)-2-{[(1R)-1-(2-methylpropanoyloxy)ethoxy]carbonylamino}-3-cyclobutyl-2-methylpropanoic acid;
(2S)-2-{[(1S)-1-(2-methylpropanoyloxy)ethoxy]carbonylamino}-3-cyclobutyl-2-methylpropanoic acid;
2-[((1R)-1-acetyloxy-2-methoxypropoxy)carbonylamino]-(2S)-3-cyclobutyl-2-methylpropanoic acid;
2-[((1S)-1-acetyloxy-2-methoxypropoxy)carbonylamino]-(2S)-3-cyclobutyl-2-methylpropanoic acid;
2-[((1R)-2-methyl-1-propanoyloxypropoxy)carbonylamino]-(2S)-3-cyclobutyl-2-methylpropanoic acid;
2-[((1S)-2-methyl-1-propanoyloxypropoxy)carbonylamino]-(2S)-3-cyclobutyl-2-methylpropanoic acid;
2-{[(1R)-2-methyl-1-(2-methylpropanoyloxy)propoxy]carbonylamino}-(2S)-3-cyclobutyl-2-methylpropanoic acid;
2-{[(1S)-2-methyl-1-(2-methylpropanoyloxy)propoxy]carbonylamino}-(2S)-3-cyclobutyl-2-methylpropanoic acid; and

a pharmaceutically acceptable salt of any of the foregoing.

In certain embodiments of a compound of Formula (II), the compound is selected from:
(2S)-2-[((1R)-1-acetyloxyethoxy)carbonylamino]-4-ethyl-2-methylhexanoic acid;
(2S)-2-[((1S)-1-acetyloxyethoxy)carbonylamino]-4-ethyl-2-methylhexanoic acid;
(2S)-2-[((1R)-1-propanoyloxyethoxy)carbonylamino]-4-ethyl-2-methylhexanoic acid;
(2S)-2-[((1S)-1-propanoyloxyethoxy)carbonylamino]-4-ethyl-2-methylhexanoic acid;
(2S)-2-{[(1R)-1-(2-methylpropanoyloxy)ethoxy]carbonylamino}-4-ethyl-2-methylhexanoic acid;
(2S)-2-{[(1S)-1-(2-methylpropanoyloxy)ethoxy]carbonylamino}-4-ethyl-2-methylhexanoic acid;

2-[((1R)-1-acetyloxy-2-methoxypropoxy)carbonylamino](2S)-4-ethyl-2-methylhexanoic acid;
2-[((1S)-1-acetyloxy-2-methoxypropoxy)carbonylamino](2S)-4-ethyl-2-methylhexanoic acid;
2-[((1R)-2-methyl-1-propanoyloxypropoxy)carbonylamino](2S)-4-ethyl-2-methylhexanoic acid;
2-[((1S)-2-methyl-1-propanoyloxypropoxy)carbonylamino](2S)-4-ethyl-2-methylhexanoic acid;
2-{[(1R)-2-methyl-1-(2-methylpropanoyloxy)propoxy]carbonylamino}(2S)-4-ethyl-2-methylhexanoic acid;
2-{[(1S)-2-methyl-1-(2-methylpropanoyloxy)propoxy]carbonylamino}(2S)-4-ethyl-2-methylhexanoic acid; and

a pharmaceutically acceptable salt of any of the foregoing.

In certain embodiments of a compound of Formula (II), the compound is selected from:
(2S)-2-[((1R)-1-acetyloxyethoxy)carbonylamino]-5-ethyl-2-methylheptanoic acid;
(2S)-2-[((1S)-1-acetyloxyethoxy)carbonylamino]-5-ethyl-2-methylheptanoic acid;
(2S)-2-[((1R)-1-propanoyloxyethoxy)carbonylamino]-5-ethyl-2-methylheptanoic acid;
(2S)-2-[((1S)-1-propanoyloxyethoxy)carbonylamino]-5-ethyl-2-methylheptanoic acid;
(2S)-2-{[(1R)-1-(2-methylpropanoyloxy)ethoxy]carbonylamino}-5-ethyl-2-methylheptanoic acid;
(2S)-2-{[(1S)-1-(2-methylpropanoyloxy)ethoxy]carbonylamino}-5-ethyl-2-methylheptanoic acid;

2-[((1R)-1-acetyloxy-2-methoxypropoxy)carbonylamino](2S)-5-ethyl-2-methylheptanoic acid;
2-[((1S)-1-acetyloxy-2-methoxypropoxy)carbonylamino](2S)-5-ethyl-2-methylheptanoic acid;
2-[((1R)-2-methyl-1-propanoyloxypropoxy)carbonylamino](2S)-5-ethyl-2-methylheptanoic acid;
2-[((1S)-2-methyl-1-propanoyloxypropoxy)carbonylamino](2S)-5-ethyl-2-methylheptanoic acid;
2-{[(1R)-2-methyl-1-(2-methylpropanoyloxy)propoxy]carbonylamino}(2S)-5-ethyl-2-methylheptanoic acid;
2-{[(1S)-2-methyl-1-(2-methylpropanoyloxy)propoxy]carbonylamino}(2S)-5-ethyl-2-methylheptanoic acid; and

a pharmaceutically acceptable salt of any of the foregoing.

In certain embodiments of a compound of Formula (II), the compound is selected from:
2-[((1R)-1-acetyloxyethoxy)carbonylamino](2S,5R)-2,5-dimethylheptanoic acid;
2-[((1S)-1-acetyloxyethoxy)carbonylamino](2S,5R)-2,5-dimethylheptanoic acid;
2-[((1R)-1-propanoyloxyethoxy)carbonylamino](2S,5R)-2,5-dimethylheptanoic acid;
2-[((1S)-1-propanoyloxyethoxy)carbonylamino](2S,5R)-2,5-dimethylheptanoic acid;
2-{[(1R)-1-(2-methylpropanoyloxy)ethoxy]carbonylamino}(2S,5R)-2,5-dimethylheptanoic acid;
2-{[(1S)-1-(2-methylpropanoyloxy)ethoxy]carbonylamino}(2S,5R)-2,5-dimethylheptanoic acid;
2-[((1R)-1-acetyloxy-2-methoxypropoxy)carbonylamino](2S,5R)-2,5-dimethylheptanoic acid;
2-[((1S)-1-acetyloxy-2-methoxypropoxy)carbonylamino](2S,5R)-2,5-dimethylheptanoic acid;
2-[((1R)-2-methyl-1-propanoyloxypropoxy)carbonylamino](2S,5R)-2,5-dimethylheptanoic acid;
2-[((1S)-2-methyl-1-propanoyloxypropoxy)carbonylamino](2S,5R)-2,5-dimethylheptanoic acid;
2-{[(1R)-2-methyl-1-(2-methylpropanoyloxy)propoxy]carbonylamino}(2S,5R)-2,5-dimethylheptanoic acid;
2-{[(1S)-2-methyl-1-(2-methylpropanoyloxy)propoxy]carbonylamino}(2S,5R)-2,5-dimethylheptanoic acid; and

a pharmaceutically acceptable salt of any of the foregoing.

Certain embodiments provide a compound of Formula (III):

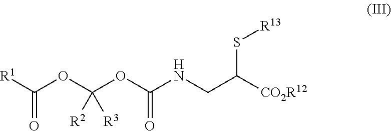

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and substituted C₃₋₈ heterocycloalkyl ring;

R¹² is selected from hydrogen and C₁₋₆ alkyl; and

R¹³ is selected from C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₅₋₁₀ heteroalkyl, substituted C₅₋₁₀ heteroalkyl, C₅₋₁₀ heteroaryl, substituted C₅₋₁₀ heteroaryl, C₅₋₁₀ heteroarylalkyl, and substituted C₅₋₁₀ heteroarylalkyl.

In certain embodiments of a compound of Formula (III), R¹² is hydrogen; and R¹³ is selected from C₁₋₅ alkyl, phenyl, halo-substituted phenyl, and C₅₋₆ cycloalkyl.

In certain embodiments of a compound of Formula (III) the moiety:

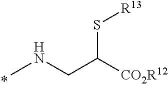

is derived from an alpha-2-delta ligand of the formula:

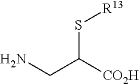

by replacing a hydrogen of the amine group with a covalent bond and by replacing the hydrogen atom of the carboxylic acid group with —R¹². In certain embodiments of a compound of Formula (III), in the alpha-2-delta ligand of the above formula, R¹² is hydrogen; and R¹³ is selected from C₁₋₅ alkyl, phenyl, halo-substituted phenyl, and C₅₋₆ cycloalkyl. In certain embodiments of a compound of Formula (III), the alpha-2-delta ligand is selected from:
(2S)-3-amino-2-(1-ethylpropylthio)-propanoic acid;
(2S)-3-amino-2-(3-chlorophenylthio)-propanoic acid;
(2S)-3-amino-2-(1-methylethylthio)-propanoic acid;
(2S)-3-amino-2-(tert-butylthio)-propanoic acid;
(2S)-3-amino-2-(2-methylpropylthio)-propanoic acid;
(2S)-3-amino-2-(2-ethylbutylthio)-propanoic acid;
(2S)-3-amino-2-(cyclopentylthio)-propanoic acid; and
(2S)-3-amino-2-(cyclohexylthio)-propanoic acid.

In certain embodiments of a compound of Formula (III), the compound is selected from:
(2S)-3-{[(1R)-1-(1-acetyloxy)ethoxy]carbonylamino}-2-(ethylpropylthio)propanoic acid;
(2S)-3-{[(1S)-1-(1-acetyloxy)ethoxy]carbonylamino}-2-(ethylpropylthio)propanoic acid;
(2S)-3-{[(1R)-1-(1-propanoyloxy)ethoxy]carbonylamino}-2-(ethylpropylthio)propanoic acid;
(2S)-3-{[(1S)-1-(1-propanoyloxy)ethoxy]carbonylamino}-2-(ethylpropylthio)propanoic acid;
(2S)-3-{[(1R)-1-(2-methylpropanoyloxy)ethoxy]carbonylamino}-2-(ethylpropylthio)propanoic acid;
(2S)-3-{[(1S)-1-(2-methylpropanoyloxy)ethoxy]carbonylamino}-2-(ethylpropylthio)propanoic acid;
(2S)-3-{[(1R)-2-methyl-1-(1-acetyloxy)propoxy]carbonylamino}-2-(ethylpropylthio)propanoic acid;
(2S)-3-{[(1S)-2-methyl-1-(1-acetyloxy)propoxy]carbonylamino}-2-(ethylpropylthio)propanoic acid;
(2S)-3-{[(1R)-2-methyl-1-(1-propanoyloxy)propoxy]carbonylamino}-2-(ethylpropylthio)propanoic acid;
(2S)-3-{[(1S)-2-methyl-1-(1-propanoyloxy)propoxy]carbonylamino}-2-(ethylpropylthio)propanoic acid;
(2S)-3-{[(1R)-2-methyl-1-(2-methylpropanoyloxy)propoxy]carbonylamino}-2-(ethylpropylthio)propanoic acid;
(2S)-3-{[(1S)-2-methyl-1-(2-methylpropanoyloxy)propoxy]carbonylamino}-2-(ethylpropylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(3-chlorophenylthio)propanoic acid;
(2S)-3-{[(1S)-1-(1-acetyloxy)ethoxy]carbonylamino}-2-(3-chlorophenylthio)propanoic acid;
(2S)-3-{[(1R)-1-(1-propanoyloxy)ethoxy]carbonylamino}-2-(3-chlorophenylthio)propanoic acid;
(2S)-3-{[(1S)-1-(1-propanoyloxy)ethoxy]carbonylamino}-2-(3-chlorophenylthio)propanoic acid;
(2S)-3-{[(1R)-1-(2-methylpropanoyloxy)ethoxy]carbonylamino}-2-(3-chlorophenylthio)propanoic acid;
(2S)-3-{[(1S)-1-(2-methylpropanoyloxy)ethoxy]carbonylamino}-2-(3-chlorophenylthio)propanoic acid;
(2S)-3-{[(1R)-2-methyl-1-(1-acetyloxy)propoxy]carbonylamino}-2-(3-chlorophenylthio)propanoic acid;
(2S)-3-{[(1S)-2-methyl-1-(1-acetyloxy)propoxy]carbonylamino}-2-(3-chlorophenylthio)propanoic acid;
(2S)-3-{[(1R)-2-methyl-1-(1-propanoyloxy)propoxy]carbonylamino}-2-(3-chlorophenylthio)propanoic acid;
(2S)-3-{[(1S)-2-methyl-1-(1-propanoyloxy)propoxy]carbonylamino}-2-(3-chlorophenylthio)propanoic acid;
(2S)-3-{[(1R)-2-methyl-1-(2-methylpropanoyloxy)propoxy]carbonylamino}-2-(3-chlorophenylthio)propanoic acid;
(2S)-3-{[(1S)-2-methyl-1-(2-methylpropanoyloxy)propoxy]carbonylamino}-2-(3-chlorophenylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(1-methylethylthio)propanoic acid;
(2S)-3-{[(1S)-1-(1-acetyloxy)ethoxy]carbonylamino}-2-(1-methylethylthio)propanoic acid;
(2S)-3-{[(1R)-1-(1-propanoyloxy)ethoxy]carbonylamino}-2-(1-methylethylthio)propanoic acid;
(2S)-3-{[(1S)-1-(1-propanoyloxy)ethoxy]carbonylamino}-2-(1-methylethylthio)propanoic acid;
(2S)-3-{[(1R)-1-(2-methylpropanoyloxy)ethoxy]carbonylamino}-2-(1-methylethylthio)propanoic acid;
(2S)-3-{[(1S)-1-(2-methylpropanoyloxy)ethoxy]carbonylamino}-2-(1-methylethylthio)propanoic acid;
(2S)-3-{[(1R)-2-methyl-1-(1-acetyloxy)propoxy]carbonylamino}-2-(1-methylethylthio)propanoic acid;
(2S)-3-{[(1S)-2-methyl-1-(1-acetyloxy)propoxy]carbonylamino}-2-(1-methylethylthio)propanoic acid;
(2S)-3-{[(1R)-2-methyl-1-(1-propanoyloxy)propoxy]carbonylamino}-2-(1-methylethylthio)propanoic acid;
(2S)-3-{[(1S)-2-methyl-1-(1-propanoyloxy)propoxy]carbonylamino}-2-(1-methylethylthio)propanoic acid;
(2S)-3-{[(1R)-2-methyl-1-(2-methylpropanoyloxy)propoxy]carbonylamino}-2-(1-methylethylthio)propanoic acid;
(2S)-3-{[(1S)-2-methyl-1-(2-methylpropanoyloxy)propoxy]carbonylamino}-2-(1-methylethylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(tert-butylthio)propanoic acid;
(2S)-3-{[(1S)-1-(1-acetyloxy)ethoxy]carbonylamino}-2-(tert-butylthio)propanoic acid;
(2S)-3-{[(1R)-1-(1-propanoyloxy)ethoxy]carbonylamino}-2-(tert-butylthio)propanoic acid;
(2S)-3-{[(1S)-1-(1-propanoyloxy)ethoxy]carbonylamino}-2-(tert-butylthio)propanoic acid;
(2S)-3-{[(1R)-1-(2-methylpropanoyloxy)ethoxy]carbonylamino}-2-(tert-butylthio)propanoic acid;
(2S)-3-{[(1S)-1-(2-methylpropanoyloxy)ethoxy]carbonylamino}-2-(tert-butylthio)propanoic acid;
(2S)-3-{[(1R)-2-methyl-1-(1-acetyloxy)propoxy]carbonylamino}-2-(tert-butylthio)propanoic acid;
(2S)-3-{[(1S)-2-methyl-1-(1-acetyloxy)propoxy]carbonylamino}-2-(tert-butylthio)propanoic acid;
(2S)-3-{[(1R)-2-methyl-1-(1-propanoyloxy)propoxy]carbonylamino}-2-(tert-butylthio)propanoic acid;
(2S)-3-{[(1S)-2-methyl-1-(1-propanoyloxy)propoxy]carbonylamino}-2-(tert-butylthio)propanoic acid;
(2S)-3-{[(1R)-2-methyl-1-(2-methylpropanoyloxy)propoxy]carbonylamino}-2-(tert-butylthio)propanoic acid;
(2S)-3-{[(1S)-2-methyl-1-(2-methylpropanoyloxy)propoxy]carbonylamino}-2-(tert-butylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(2-methylpropylthio)propanoic acid;
(2S)-3-{[(1S)-1-(1-acetyloxy)ethoxy]carbonylamino}-2-(2-methylpropylthio)propanoic acid;
(2S)-3-{[(1R)-1-(1-propanoyloxy)ethoxy]carbonylamino}-2-(2-methylpropylthio)propanoic acid;
(2S)-3-{[(1S)-1-(1-propanoyloxy)ethoxy]carbonylamino}-2-(2-methylpropylthio)propanoic acid;
(2S)-3-{[(1R)-1-(2-methylpropanoyloxy)ethoxy]carbonylamino}-2-(2-methylpropylthio)propanoic acid;
(2S)-3-{[(1S)-1-(2-methylpropanoyloxy)ethoxy]carbonylamino}-2-(2-methylpropylthio)propanoic acid;
(2S)-3-{[(1R)-2-methyl-1-(1-acetyloxy)propoxy]carbonylamino}-2-(2-methylpropylthio)propanoic acid;
(2S)-3-{[(1S)-2-methyl-1-(1-acetyloxy)propoxy]carbonylamino}-2-(2-methylpropylthio)propanoic acid;
(2S)-3-{[(1R)-2-methyl-1-(1-propanoyloxy)propoxy]carbonylamino}-2-(2-methylpropylthio)propanoic acid;
(2S)-3-{[(1S)-2-methyl-1-(1-propanoyloxy)propoxy]carbonylamino}-2-(2-methylpropylthio)propanoic acid;
(2S)-3-{[(1R)-2-methyl-1-(2-methylpropanoyloxy)propoxy]carbonylamino}-2-(2-methylpropylthio)propanoic acid;
(2S)-3-{[(1S)-2-methyl-1-(2-methylpropanoyloxy)propoxy]carbonylamino}-2-(2-methylpropylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(2-ethylbutylthio)propanoic acid;
(2S)-3-{[(1S)-1-(1-acetyloxy)ethoxy]carbonylamino}-2-(2-ethylbutylthio)propanoic acid;
(2S)-3-{[(1R)-1-(1-propanoyloxy)ethoxy]carbonylamino}-2-(2-ethylbutylthio)propanoic acid;
(2S)-3-{[(1S)-1-(1-propanoyloxy)ethoxy]carbonylamino}-2-(2-ethylbutylthio)propanoic acid;
(2S)-3-{[(1R)-1-(2-methylpropanoyloxy)ethoxy]carbonylamino}-2-(2-ethylbutylthio)propanoic acid;
(2S)-3-{[(1S)-1-(2-methylpropanoyloxy)ethoxy]carbonylamino}-2-(2-ethylbutylthio)propanoic acid;
(2S)-3-{[(1R)-2-methyl-1-(1-acetyloxy)propoxy]carbonylamino}-2-(2-ethylbutylthio)propanoic acid;
(2S)-3-{[(1S)-2-methyl-1-(1-acetyloxy)propoxy]carbonylamino}-2-(2-ethylbutylthio)propanoic acid;
(2S)-3-{[(1R)-2-methyl-1-(1-propanoyloxy)propoxy]carbonylamino}-2-(2-ethylbutylthio)propanoic acid;
(2S)-3-{[(1S)-2-methyl-1-(1-propanoyloxy)propoxy]carbonylamino}-2-(2-ethylbutylthio)propanoic acid;
(2S)-3-{[(1R)-2-methyl-1-(2-methylpropanoyloxy)propoxy]carbonylamino}-2-(2-ethylbutylthio)propanoic acid;
(2S)-3-{[(1S)-2-methyl-1-(2-methylpropanoyloxy)propoxy]carbonylamino}-2-(2-ethylbutylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(cyclopentylthio)propanoic acid;
(2S)-3-{[(1S)-1-(1-acetyloxy)ethoxy]carbonylamino}-2-(cyclopentylthio)propanoic acid;
(2S)-3-{[(1R)-1-(1-propanoyloxy)ethoxy]carbonylamino}-2-(cyclopentylthio)propanoic acid;
(2S)-3-{[(1S)-1-(1-propanoyloxy)ethoxy]carbonylamino}-2-(cyclopentylthio)propanoic acid;
(2S)-3-{[(1R)-1-(2-methylpropanoyloxy)ethoxy]carbonylamino}-2-(cyclopentylthio)propanoic acid;
(2S)-3-{[(1S)-1-(2-methylpropanoyloxy)ethoxy]carbonylamino}-2-(cyclopentylthio)propanoic acid;
(2S)-3-{[(1R)-2-methyl-1-(1-acetyloxy)propoxy]carbonylamino}-2-(cyclopentylthio)propanoic acid;
(2S)-3-{[(1S)-2-methyl-1-(1-acetyloxy)propoxy]carbonylamino}-2-(cyclopentylthio)propanoic acid;
(2S)-3-{[(1R)-2-methyl-1-(1-propanoyloxy)propoxy]carbonylamino}-2-(cyclopentylthio)propanoic acid;
(2S)-3-{[(1S)-2-methyl-1-(1-propanoyloxy)propoxy]carbonylamino}-2-(cyclopentylthio)propanoic acid;
(2S)-3-{[(1R)-2-methyl-1-(2-methylpropanoyloxy)propoxy]carbonylamino}-2-(cyclopentylthio)propanoic acid;
(2S)-3-{[(1S)-2-methyl-1-(2-methylpropanoyloxy)propoxy]carbonylamino}-2-(cyclopentylthio)propanoic acid; and

a pharmaceutically acceptable salt of any of the foregoing.

In certain embodiments of a compound of Formula (III), the compound is selected from:
(2S)-3-{[(1R)-1-(1-acetyloxy)ethoxy]carbonylamino}-2-(cyclohexylthio)propanoic acid;
(2S)-3-{[(1S)-1-(1-acetyloxy)ethoxy]carbonylamino}-2-(cyclohexylthio)propanoic acid;
(2S)-3-{[(1R)-1-(1-propanoyloxy)ethoxy]carbonylamino}-2-(cyclohexylthio)propanoic acid;
(2S)-3-{[(1S)-1-(1-propanoyloxy)ethoxy]carbonylamino}-2-(cyclohexylthio)propanoic acid;
(2S)-3-{[(1R)-1-(2-methylpropanoyloxy)ethoxy]carbonylamino}-2-(cyclohexylthio)propanoic acid;
(2S)-3-{[(1S)-1-(2-methylpropanoyloxy)ethoxy]carbonylamino}-2-(cyclohexylthio)propanoic acid;
(2S)-3-{[(1R)-2-methyl-1-(1-acetyloxy)propoxy]carbonylamino}-2-(cyclohexylthio)propanoic acid;
(2S)-3-{[(1S)-2-methyl-1-(1-acetyloxy)propoxy]carbonylamino}-2-(cyclohexylthio)propanoic acid;
(2S)-3-{[(1R)-2-methyl-1-(1-propanoyloxy)propoxy]carbonylamino}-2-(cyclohexylthio)propanoic acid;
(2S)-3-{[(1S)-2-methyl-1-(1-propanoyloxy)propoxy]carbonylamino}-2-(cyclohexylthio)propanoic acid;
(2S)-3-{[(1R)-2-methyl-1-(2-methylpropanoyloxy)propoxy]carbonylamino}-2-(cyclohexylthio)propanoic acid;
(2S)-3-{[(1S)-2-methyl-1-(2-methylpropanoyloxy)propoxy]carbonylamino}-2-(cyclohexylthio)propanoic acid; and

a pharmaceutically acceptable salt of any of the foregoing.

Certain embodiments provide a compound of Formula (IV):

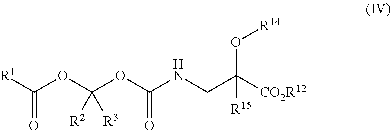

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₃₋₈ heterocycloalkyl, substituted C₃₋₈ heterocycloalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;

R¹² is selected from hydrogen and C₁₋₆ alkyl;

R¹⁴ is selected from C₆₋₁₀ aryl and substituted C₆₋₁₀ aryl; and

R¹⁵ is hydrogen or methyl.

In certain embodiments of a compound of Formula (IV), R¹² is hydrogen; R¹⁴ is selected from phenyl, halo-substituted phenyl, and C₁₋₃ alkoxy-substituted phenyl; and R¹⁵ is hydrogen.

In certain embodiments of a compound of Formula (IV) the moiety:

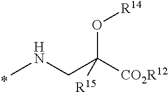

is derived from an alpha-2-delta ligand of the formula:

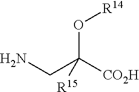

by replacing a hydrogen of the amine group with a covalent bond and by replacing the hydrogen atom of the carboxylic acid group with —R¹². In certain embodiments of a compound of Formula (IV), in the alpha-2-delta ligand of the above formula, R¹² is hydrogen; R¹⁴ is selected from phenyl, halo-substituted phenyl, and C₁₋₃ alkoxy-substituted phenyl; and R¹⁵ is hydrogen. In certain embodiments of a compound of Formula (IV), the alpha-2-delta ligand is selected from:
(2S)-3-amino-2-(2,5-dichlorophenoxy)propanoic acid;
(2S)-3-amino-2-(3-chlorophenoxy)propanoic acid;
(2S)-3-amino-2-(2-chlorophenoxy)propanoic acid;
(2S)-3-amino-2-(2-methoxy-5-chlorophenoxy)propanoic acid; and
(2S)-3-amino-2-(3-propylphenoxy)propanoic acid.

In certain embodiments of a compound of Formula (IV), the compound is selected from:
3-{[(1R)-1-(1-acetyloxy)ethoxy]carbonylamino}(2S)-2-(2,5-dichlorophenoxy)propanoic acid;
3-{[(1S)-1-(1-acetyloxy)ethoxy]carbonylamino}(2S)-2-(2,5-dichlorophenoxy)propanoic acid;
3-{[(1R)-1-(1-propanoyloxy)ethoxy]carbonylamino}(2S)-2-(2,5-dichlorophenoxy)propanoic acid;
3-{[(1S)-1-(1-propanoyloxy)ethoxy]carbonylamino}(2S)-2-(2,5-dichlorophenoxy)propanoic acid;
3-{[(1R)-1-(2-methylpropanoyloxy)ethoxy]carbonylamino}(2S)-2-(2,5-dichlorophenoxy)propanoic acid;
3-{[(1S)-1-(2-methylpropanoyloxy)ethoxy]carbonylamino}(2S)-2-(2,5-dichlorophenoxy)propanoic acid;
3-{[(1R)-2-methyl-1-(1-acetyloxy)propoxy]carbonylamino}(2S)-2-(2,5-dichlorophenoxy)propanoic acid;
3-{[(1S)-2-methyl-1-(1-acetyloxy)propoxy]carbonylamino}(2S)-2-(2,5-dichlorophenoxy)propanoic acid;
3-{[(1R)-2-methyl-1-(1-propanoyloxy)propoxy]carbonylamino}(2S)-2-(2,5-dichlorophenoxy)propanoic acid;
3-{[(1S)-2-methyl-1-(1-propanoyloxy)propoxy]carbonylamino}(2S)-2-(2,5-dichlorophenoxy)propanoic acid;
3-{[(1R)-2-methyl-1-(2-methylpropanoyloxy)propoxy]carbonylamino}(2S)-2-(2,5-dichlorophenoxy)propanoic acid;
3-{[(1S)-2-methyl-1-(2-methylpropanoyloxy)propoxy]carbonylamino}(2S)-2-(2,5-dichlorophenoxy)propanoic acid; and

a pharmaceutically acceptable salt of any of the foregoing.

In certain embodiments of a compound of Formula (IV), the compound is selected from:
3-{[(1R)-1-(1-acetyloxy)ethoxy]carbonylamino}(2S)-2-(3-chlorophenoxy)propanoic acid;
3-{[(1S)-1-(1-acetyloxy)ethoxy]carbonylamino}(2S)-2-(3-chlorophenoxy)propanoic acid;
3-{[(1R)-1-(1-propanoyloxy)ethoxy]carbonylamino}(2S)-2-(3-chlorophenoxy)propanoic acid;
3-{[(1S)-1-(1-propanoyloxy)ethoxy]carbonylamino}(2S)-2-(3-chlorophenoxy)propanoic acid;
3-{[(1R)-1-(2-methylpropanoyloxy)ethoxy]carbonylamino}(2S)-2-(3-chlorophenoxy)propanoic acid;
3-{[(1S)-1-(2-methylpropanoyloxy)ethoxy]carbonylamino}(2S)-2-(3-chlorophenoxy)propanoic acid;
3-{[(1R)-2-methyl-1-(1-acetyloxy)propoxy]carbonylamino}(2S)-2-(3-chlorophenoxy)propanoic acid;
3-{[(1S)-2-methyl-1-(1-acetyloxy)propoxy]carbonylamino}(2S)-2-(3-chlorophenoxy)propanoic acid;
3-{[(1R)-2-methyl-1-(1-propanoyloxy)propoxy]carbonylamino}(2S)-2-(3-chlorophenoxy)propanoic acid;
3-{[(1S)-2-methyl-1-(1-propanoyloxy)propoxy]carbonylamino}(2S)-2-(3-chlorophenoxy)propanoic acid;
3-{[(1R)-2-methyl-1-(2-methylpropanoyloxy)propoxy]carbonylamino}(2S)-2-(3-chlorophenoxy)propanoic acid;
3-{[(1S)-2-methyl-1-(2-methylpropanoyloxy)propoxy]carbonylamino}(2S)-2-(3-chlorophenoxy)propanoic acid; and a pharmaceutically acceptable salt of any of the foregoing.

In certain embodiments of a compound of Formula (IV), the compound is selected from:
3-{[(1R)-1-(1-acetyloxy)ethoxy]carbonylamino}(2S)-2-(2-chlorophenoxy)propanoic acid;
3-{[(1S)-1-(1-acetyloxy)ethoxy]carbonylamino}(2S)-2-(2-chlorophenoxy)propanoic acid;
3-{[(1R)-1-(1-propanoyloxy)ethoxy]carbonylamino}(2S)-2-(2-chlorophenoxy)propanoic acid;
3-{[(1S)-1-(1-propanoyloxy)ethoxy]carbonylamino}(2S)-2-(2-chlorophenoxy)propanoic acid;
3-{[(1R)-1-(2-methylpropanoyloxy)ethoxy]carbonylamino}(2S)-2-(2-chlorophenoxy)propanoic acid;
3-{[(1S)-1-(2-methylpropanoyloxy)ethoxy]carbonylamino}(2S)-2-(2-chlorophenoxy)propanoic acid;
3-{[(1R)-2-methyl-1-(1-acetyloxy)propoxy]carbonylamino}(2S)-2-(2-chlorophenoxy)propanoic acid;
3-{[(1S)-2-methyl-1-(1-acetyloxy)propoxy]carbonylamino}(2S)-2-(2-chlorophenoxy)propanoic acid;
3-{[(1R)-2-methyl-1-(1-propanoyloxy)propoxy]carbonylamino}(2S)-2-(2-chlorophenoxy)propanoic acid;
3-{[(1S)-2-methyl-1-(1-propanoyloxy)propoxy]carbonylamino}(2S)-2-(2-chlorophenoxy)propanoic acid;
3-{[(1R)-2-methyl-1-(2-methylpropanoyloxy)propoxy]carbonylamino}(2S)-2-(2-chlorophenoxy)propanoic acid;
3-{[(1S)-2-methyl-1-(2-methylpropanoyloxy)propoxy]carbonylamino}(2S)-2-(2-chlorophenoxy)propanoic acid; and

a pharmaceutically acceptable salt of any of the foregoing.

In certain embodiments of a compound of Formula (IV), the compound is selected from:
3-{[(1R)-1-(1-acetyloxy)ethoxy]carbonylamino}(2S)-2-(2-methoxy-5-chlorophenoxy)propanoic acid;
3-{[(1S)-1-(1-acetyloxy)ethoxy]carbonylamino}(2S)-2-(2-methoxy-5-chlorophenoxy)propanoic acid;
3-{[(1R)-1-(1-propanoyloxy)ethoxy]carbonylamino}(2S)-2-(2-methoxy-5-chlorophenoxy)propanoic acid;
3-{[(1S)-1-(1-propanoyloxy)ethoxy]carbonylamino}(2S)-2-(2-methoxy-5-chlorophenoxy)propanoic acid;
3-{[(1R)-1-(2-methylpropanoyloxy)ethoxy]carbonylamino}(2S)-2-(2-methoxy-5-chlorophenoxy)propanoic acid;
3-{[(1S)-1-(2-methylpropanoyloxy)ethoxy]carbonylamino}(2S)-2-(2-methoxy-5-chlorophenoxy)propanoic acid;
3-{[(1R)-2-methyl-1-(1-acetyloxy)propoxy]carbonylamino}(2S)-2-(2-methoxy-5-chlorophenoxy)propanoic acid;
3-{[(1S)-2-methyl-1-(1-acetyloxy)propoxy]carbonylamino}(2S)-2-(2-methoxy-5-chlorophenoxy)propanoic acid;
3-{[(1R)-2-methyl-1-(1-propanoyloxy)propoxy]carbonylamino}(2S)-2-(2-methoxy-5-chlorophenoxy)propanoic acid;
3-{[(1S)-2-methyl-1-(1-propanoyloxy)propoxy]carbonylamino}(2S)-2-(2-methoxy-5-chlorophenoxy)propanoic acid;
3-{[(1R)-2-methyl-1-(2-methylpropanoyloxy)propoxy]carbonylamino}(2S)-2-(2-methoxy-5-chlorophenoxy)propanoic acid;
3-{[(1S)-2-methyl-1-(2-methylpropanoyloxy)propoxy]carbonylamino}(2S)-2-(2-methoxy-5-chlorophenoxy)propanoic acid; and

a pharmaceutically acceptable salt of any of the foregoing.

In certain embodiments of a compound of Formula (IV), the compound is selected from:
3-{[(1R)-1-(1-acetyloxy)ethoxy]carbonylamino}(2S)-2-(3-propylphenoxy)propanoic acid;
3-{[(1S)-1-(1-acetyloxy)ethoxy]carbonylamino}(2S)-2-(3-propylphenoxy)propanoic acid;
3-{[(1R)-1-(1-propanoyloxy)ethoxy]carbonylamino}(2S)-2-(3-propylphenoxy)propanoic acid;
3-{[(1S)-1-(1-propanoyloxy)ethoxy]carbonylamino}(2S)-2-(3-propylphenoxy)propanoic acid;
3-{[(1R)-1-(2-methylpropanoyloxy)ethoxy]carbonylamino}(2S)-2-(3-propylphenoxy)propanoic acid;
3-{[(1S)-1-(2-methylpropanoyloxy)ethoxy]carbonylamino}(2S)-2-(3-propylphenoxy)propanoic acid;
3-{[(1R)-2-methyl-1-(1-acetyloxy)propoxy]carbonylamino}(2S)-2-(3-propylphenoxy)propanoic acid;
3-{[(1S)-2-methyl-1-(1-acetyloxy)propoxy]carbonylamino}(2S)-2-(3-propylphenoxy)propanoic acid;
3-{[(1R)-2-methyl-1-(1-propanoyloxy)propoxy]carbonylamino}(2S)-2-(3-propylphenoxy)propanoic acid;
3-{[(1S)-2-methyl-1-(1-propanoyloxy)propoxy]carbonylamino}(2S)-2-(3-propylphenoxy)propanoic acid;
3-{[(1R)-2-methyl-1-(2-methylpropanoyloxy)propoxy]carbonylamino}(2S)-2-(3-propylphenoxy)propanoic acid;
3-{[(1S)-2-methyl-1-(2-methylpropanoyloxy)propoxy]carbonylamino}(2S)-2-(3-propylphenoxy)propanoic acid; and

a pharmaceutically acceptable salt of any of the foregoing.

Certain embodiments provide a compound of Formula (V):

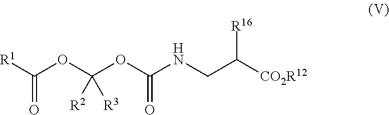

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, substituted C₅₋₁₀ heteroarylalkyl, C₁₋₈ heterocycloalkyl, and substituted C₁₋₈ heterocycloalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;

R¹² is selected from hydrogen and C₁₋₆ alkyl; and

R¹⁶ is selected from C₇₋₁₈ arylalkyl and substituted C₇₋₁₈ arylalkyl.

In certain embodiments of a compound of Formula (V), R¹² is hydrogen; and R¹⁶ is selected from benzyl, substituted benzyl wherein the one or more substituents is selected from halo, trifluoromethyl, C₁₋₄ alkyl, and C₁₋₄ thioalkyl, phenyl, and halo-substituted phenyl.

In certain embodiments of a compound of Formula (V) the moiety:

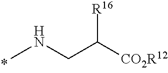

is derived from an alpha-2-delta ligand of the formula:

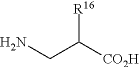

by replacing a hydrogen of the amine group with a covalent bond and by replacing the hydrogen atom of the carboxylic acid group with —R¹². In certain embodiments of a compound of Formula (V), in the alpha-2-delta ligand of the above formula, R¹² is hydrogen; and R¹⁶ is selected from benzyl, substituted benzyl wherein the one or more substituents is selected from halo, trifluoromethyl, C₁₋₄ alkyl, and C₁₋₄ thioalkyl, phenyl, and halo-substituted phenyl. In certain embodiments of a compound of Formula (V), the alpha-2-delta ligand is selected from:
(2S)-3-amino-2-[(3-chlorophenyl)methyl]propanoic acid;
(2S)-3-amino-2-[(2,5-dichlorophenyl)methyl]propanoic acid;
(2S)-3-amino-2-[(3-trifluoromethylphenyl)methyl]propanoic acid;
(2S)-3-amino-2-[(3-isobutylphenyl)methyl]propanoic acid;
(2S)-3-amino-2-[(3,5-dichlorophenyl)methyl]propanoic acid;
(2S)-3-amino-2-[(3-methylthiophenyl)methyl]propanoic acid;
(2S)-3-amino-2-[(3-bromophenyl)methyl]propanoic acid; and
(2S)-3-amino-2-{[2-(4-fluorophenyl)phenyl]methyl}-propanoic acid.

In certain embodiments of a compound of Formula (V), the compound is selected from:
3-{[(1R)-1-(1-acetyloxy)ethoxy]carbonylamino}(2S)-2-[(3-chlorophenyl)methyl]propanoic acid;
3-{[(1S)-1-(1-acetyloxy)ethoxy]carbonylamino}(2S)-2-[(3-chlorophenyl)methyl]propanoic acid;
3-{[(1R)-1-(1-propanoyloxy)ethoxy]carbonylamino}(2S)-2-[(3-chlorophenyl)methyl]propanoic acid;
3-{[(1S)-1-(1-propanoyloxy)ethoxy]carbonylamino}(2S)-2-[(3-chlorophenyl)methyl]propanoic acid;
3-{[(1R)-1-(2-methylpropanoyloxy)ethoxy]carbonylamino}(2S)-2-[(3-chlorophenyl)methyl]propanoic acid;
3-{[(1S)-1-(2-methylpropanoyloxy)ethoxy]carbonylamino}(2S)-2-[(3-chlorophenyl)methyl]propanoic acid;
3-{[(1R)-2-methyl-1-(1-acetyloxy)propoxy]carbonylamino}(2S)-2-[(3-chlorophenyl)methyl]propanoic acid;
3-{[(1S)-2-methyl-1-(1-acetyloxy)propoxy]carbonylamino}(2S)-2-[(3-chlorophenyl)methyl]propanoic acid;
3-{[(1R)-2-methyl-1-(1-propanoyloxy)propoxy]carbonylamino}(2S)-2-[(3-chlorophenyl)methyl]propanoic acid;
3-{[(1S)-2-methyl-1-(1-propanoyloxy)propoxy]carbonylamino}(2S)-2-[(3-chlorophenyl)methyl]propanoic acid;
3-{[(1R)-2-methyl-1-(2-methylpropanoyloxy)propoxy]carbonylamino}(2S)-2-[(3-chlorophenyl)methyl]propanoic acid;
3-{[(1S)-2-methyl-1-(2-methylpropanoyloxy)propoxy]carbonylamino}(2S)-2-[(3-chlorophenyl)methyl]propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-[(2,5-dichlorophenyl)methyl]propanoic acid;
3-{[(1S)-1-(1-acetyloxy)ethoxy]carbonylamino}(2S)-2-[(2,5-dichlorophenyl)methyl]propanoic acid;
3-{[(1R)-1-(1-propanoyloxy)ethoxy]carbonylamino}(2S)-2-[(2,5-dichlorophenyl)methyl]propanoic acid;
3-{[(1S)-1-(1-propanoyloxy)ethoxy]carbonylamino}(2S)-2-[(2,5-dichlorophenyl)methyl]propanoic acid;
3-{[(1R)-1-(2-methylpropanoyloxy)ethoxy]carbonylamino}(2S)-2-[(2,5-dichlorophenyl)methyl]propanoic acid;
3-{[(1S)-1-(2-methylpropanoyloxy)ethoxy]carbonylamino}(2S)-2-[(2,5-dichlorophenyl)methyl]propanoic acid;
3-{[(1R)-2-methyl-1-(1-acetyloxy)propoxy]carbonylamino}(2S)-2-[(2,5-dichlorophenyl)methyl]propanoic acid;
3-{[(1S)-2-methyl-1-(1-acetyloxy)propoxy]carbonylamino}(2S)-2-[(2,5-dichlorophenyl)methyl]propanoic acid;
3-{[(1R)-2-methyl-1-(1-propanoyloxy)propoxy]carbonylamino}(2S)-2-[(2,5-dichlorophenyl)methyl]propanoic acid;
3-{[(1S)-2-methyl-1-(1-propanoyloxy)propoxy]carbonylamino}(2S)-2-[(2,5-dichlorophenyl)methyl]propanoic acid;
3-{[(1R)-2-methyl-1-(2-methylpropanoyloxy)propoxy]carbonylamino}(2S)-2-[(2,5-dichlorophenyl)methyl]propanoic acid;
3-{[(1S)-2-methyl-1-(2-methylpropanoyloxy)propoxy]carbonylamino}(2S)-2-[(2,5-dichlorophenyl)methyl]propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-{[3-(trifluoromethyl)phenyl]methyl}propanoic acid;
3-{[(1S)-1-(1-acetyloxy)ethoxy]carbonylamino}(2S)-2-{[3-(trifluoromethyl)phenyl]methyl}propanoic acid;
3-{[(1R)-1-(1-propanoyloxy)ethoxy]carbonylamino}(2S)-2-{[3-(trifluoromethyl)phenyl]methyl}propanoic acid;
3-{[(1S)-1-(1-propanoyloxy)ethoxy]carbonylamino}(2S)-2-{[3-(trifluoromethyl)phenyl]methyl}propanoic acid;
3-{[(1R)-1-(2-methylpropanoyloxy)ethoxy]carbonylamino}(2S)-2-{[3-(trifluoromethyl)phenyl]methyl}propanoic acid;
3-{[(1S)-1-(2-methylpropanoyloxy)ethoxy]carbonylamino}(2S)-2-{[3-(trifluoromethyl)phenyl]methyl}propanoic acid;
3-{[(1R)-2-methyl-1-(1-acetyloxy)propoxy]carbonylamino}(2S)-2-{[3-(trifluoromethyl)phenyl]methyl}propanoic acid;
3-{[(1S)-2-methyl-1-(1-acetyloxy)propoxy]carbonylamino}(2S)-2-{[3-(trifluoromethyl)phenyl]methyl}propanoic acid;
3-{[(1R)-2-methyl-1-(1-propanoyloxy)propoxy]carbonylamino}(2S)-2-{[3-(trifluoromethyl)phenyl]methyl}propanoic acid;
3-{[(1S)-2-methyl-1-(1-propanoyloxy)propoxy]carbonylamino}(2S)-2-{[3-(trifluoromethyl)phenyl]methyl}propanoic acid;
3-{[(1R)-2-methyl-1-(2-methylpropanoyloxy)propoxy]carbonylamino}(2S)-2-{[3-(trifluoromethyl)phenyl]methyl}propanoic acid;
3-{[(1S)-2-methyl-1-(2-methylpropanoyloxy)propoxy]carbonylamino}(2S)-2-{[3-(trifluoromethyl)phenyl]methyl}propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-{[3-(2-methylpropyl)phenyl]methyl}propanoic acid;
3-{[(1S)-1-(1-acetyloxy)ethoxy]carbonylamino}(2S)-2-{[3-(2-methylpropyl)phenyl]methyl}propanoic acid;
3-{[(1R)-1-(1-propanoyloxy)ethoxy]carbonylamino}(2S)-2-{[3-(2-methylpropyl)phenyl]methyl}propanoic acid;
3-{[(1S)-1-(1-propanoyloxy)ethoxy]carbonylamino}(2S)-2-{[3-(2-methylpropyl)phenyl]methyl}propanoic acid;
3-{[(1R)-1-(2-methylpropanoyloxy)ethoxy]carbonylamino}(2S)-2-{[3-(2-methylpropyl)phenyl]methyl}propanoic acid;
3-{[(1S)-1-(2-methylpropanoyloxy)ethoxy]carbonylamino}(2S)-2-{[3-(2-methylpropyl)phenyl]methyl}propanoic acid;
3-{[(1R)-2-methyl-1-(1-acetyloxy)propoxy]carbonylamino}(2S)-2-{[3-(2-methylpropyl)phenyl]methyl}propanoic acid;
3-{[(1S)-2-methyl-1-(1-acetyloxy)propoxy]carbonylamino}(2S)-2-{[3-(2-methylpropyl)phenyl]methyl}propanoic acid;
3-{[(1R)-2-methyl-1-(1-propanoyloxy)propoxy]carbonylamino}(2S)-2-{[3-(2-methylpropyl)phenyl]methyl}propanoic acid;
3-{[(1S)-2-methyl-1-(1-propanoyloxy)propoxy]carbonylamino}(2S)-2-{[3-(2-methylpropyl)phenyl]methyl}propanoic acid;
3-{[(1R)-2-methyl-1-(2-methylpropanoyloxy)propoxy]carbonylamino}(2S)-2-{[3-(2-methylpropyl)phenyl]methyl}propanoic acid;
3-{[(1S)-2-methyl-1-(2-methylpropanoyloxy)propoxy]carbonylamino}(2S)-2-{[3-(2-methylpropyl)phenyl]methyl}propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-[(3,5-dichlorophenyl)methyl]propanoic acid;
3-{[(1S)-1-(1-acetyloxy)ethoxy]carbonylamino}(2S)-2-[(3,5-dichlorophenyl)methyl]propanoic acid;
3-{[(1R)-1-(1-propanoyloxy)ethoxy]carbonylamino}(2S)-2-[(3,5-dichlorophenyl)methyl]propanoic acid;
3-{[(1S)-1-(1-propanoyloxy)ethoxy]carbonylamino}(2S)-2-[(3,5-dichlorophenyl)methyl]propanoic acid;
3-{[(1R)-1-(2-methylpropanoyloxy)ethoxy]carbonylamino}(2S)-2-[(3,5-dichlorophenyl)methyl]propanoic acid;
3-{[(1S)-1-(2-methylpropanoyloxy)ethoxy]carbonylamino}(2S)-2-[(3,5-dichlorophenyl)methyl]propanoic acid;
3-{[(1R)-2-methyl-1-(1-acetyloxy)propoxy]carbonylamino}(2S)-2-[(3,5-dichlorophenyl)methyl]propanoic acid;
3-{[(1S)-2-methyl-1-(1-acetyloxy)propoxy]carbonylamino}(2S)-2-[(3,5-dichlorophenyl)methyl]propanoic acid;
3-{[(1R)-2-methyl-1-(1-propanoyloxy)propoxy]carbonylamino}(2S)-2-[(3,5-dichlorophenyl)methyl]propanoic acid;
3-{[(1S)-2-methyl-1-(1-propanoyloxy)propoxy]carbonylamino}(2S)-2-[(3,5-dichlorophenyl)methyl]propanoic acid;
3-{[(1R)-2-methyl-1-(2-methylpropanoyloxy)propoxy]carbonylamino}(2S)-2-[(3,5-dichlorophenyl)methyl]propanoic acid;
3-{[(1S)-2-methyl-1-(2-methylpropanoyloxy)propoxy]carbonylamino}(2S)-2-[(3,5-dichlorophenyl)methyl]propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-[(3-methylthiophenyl)methyl]propanoic acid;
3-{[(1S)-1-(1-acetyloxy)ethoxy]carbonylamino}(2S)-2-[(3-methylthiophenyl)methyl]propanoic acid;
3-{[(1R)-1-(1-propanoyloxy)ethoxy]carbonylamino}(2S)-2-[(3-methylthiophenyl)methyl]propanoic acid;
3-{[(1S)-1-(1-propanoyloxy)ethoxy]carbonylamino}(2S)-2-[(3-methylthiophenyl)methyl]propanoic acid;
3-{[(1R)-1-(2-methylpropanoyloxy)ethoxy]carbonylamino}(2S)-2-[(3-methylthiophenyl)methyl]propanoic acid;
3-{[(1S)-1-(2-methylpropanoyloxy)ethoxy]carbonylamino}(2S)-2-[(3-methylthiophenyl)methyl]propanoic acid;
3-{[(1R)-2-methyl-1-(1-acetyloxy)propoxy]carbonylamino}(2S)-2-[(3-methylthiophenyl)methyl]propanoic acid;
3-{[(1S)-2-methyl-1-(1-acetyloxy)propoxy]carbonylamino}(2S)-2-[(3-methylthiophenyl)methyl]propanoic acid;
3-{[(1R)-2-methyl-1-(1-propanoyloxy)propoxy]carbonylamino}(2S)-2-[(3-methylthiophenyl)methyl]propanoic acid;
3-{[(1S)-2-methyl-1-(1-propanoyloxy)propoxy]carbonylamino}(2S)-2-[(3-methylthiophenyl)methyl]propanoic acid;
3-{[(1R)-2-methyl-1-(2-methylpropanoyloxy)propoxy]carbonylamino}(2S)-2-[(3-methylthiophenyl)methyl]propanoic acid;
3-{[(1S)-2-methyl-1-(2-methylpropanoyloxy)propoxy]carbonylamino}(2S)-2-[(3-methylthiophenyl)methyl]propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-[(3-bromophenyl)methyl]propanoic acid;
3-{[(1S)-1-(1-acetyloxy)ethoxy]carbonylamino}(2S)-2-[(3-bromophenyl)methyl]propanoic acid;
3-{[(1R)-1-(1-propanoyloxy)ethoxy]carbonylamino}(2S)-2-[(3-bromophenyl)methyl]propanoic acid;
3-{[(1S)-1-(1-propanoyloxy)ethoxy]carbonylamino}(2S)-2-[(3-bromophenyl)methyl]propanoic acid;
3-{[(1R)-1-(2-methylpropanoyloxy)ethoxy]carbonylamino}(2S)-2-[(3-bromophenyl)methyl]propanoic acid;
3-{[(1S)-1-(2-methylpropanoyloxy)ethoxy]carbonylamino}(2S)-2-[(3-bromophenyl)methyl]propanoic acid;
3-{[(1R)-2-methyl-1-(1-acetyloxy)propoxy]carbonylamino}(2S)-2-[(3-bromophenyl)methyl]propanoic acid;
3-{[(1S)-2-methyl-1-(1-acetyloxy)propoxy]carbonylamino}(2S)-2-[(3-bromophenyl)methyl]propanoic acid;
3-{[(1R)-2-methyl-1-(1-propanoyloxy)propoxy]carbonylamino}(2S)-2-[(3-bromophenyl)methyl]propanoic acid;
3-{[(1S)-2-methyl-1-(1-propanoyloxy)propoxy]carbonylamino}(2S)-2-[(3-bromophenyl)methyl]propanoic acid;
3-{[(1R)-2-methyl-1-(2-methylpropanoyloxy)propoxy]carbonylamino}(2S)-2-[(3-bromophenyl)methyl]propanoic acid;
3-{[(1S)-2-methyl-1-(2-methylpropanoyloxy)propoxy]carbonylamino}(2S)-2-[(3-bromophenyl)methyl]propanoic acid; and

a pharmaceutically acceptable salt of any of the foregoing.

In certain embodiments of a compound of Formula (V), the compound is selected from:
3-{[(1R)-1-(1-acetyloxy)ethoxy]carbonylamino}(2S)-2-{[2-(4-fluorophenyl)phenyl]methyl}propanoic acid;
3-{[(1S)-1-(1-acetyloxy)ethoxy]carbonylamino}(2S)-2-{[2-(4-fluorophenyl)phenyl]methyl}propanoic acid;
3-{[(1R)-1-(1-propanoyloxy)ethoxy]carbonylamino}(2S)-2-{[2-(4-fluorophenyl)phenyl]methyl}propanoic acid;
3-{[(1S)-1-(1-propanoyloxy)ethoxy]carbonylamino}(2S)-2-{[2-(4-fluorophenyl)phenyl]methyl}propanoic acid;
3-{[(1R)-1-(2-methylpropanoyloxy)ethoxy]carbonylamino}(2S)-2-{[2-(4-fluorophenyl)phenyl]methyl}propanoic acid;
3-{[(1S)-1-(2-methylpropanoyloxy)ethoxy]carbonylamino}(2S)-2-{[2-(4-fluorophenyl)phenyl]methyl}propanoic acid;
3-{[(1R)-2-methyl-1-(1-acetyloxy)propoxy]carbonylamino}(2S)-2-{[2-(4-fluorophenyl)phenyl]methyl}propanoic acid;
3-{[(1S)-2-methyl-1-(1-acetyloxy)propoxy]carbonylamino}(2S)-2-{[2-(4-fluorophenyl)phenyl]methyl}propanoic acid;
3-{[(1R)-2-methyl-1-(1-propanoyloxy)propoxy]carbonylamino}(2S)-2-{[2-(4-fluorophenyl)phenyl]methyl}propanoic acid;
3-{[(1S)-2-methyl-1-(1-propanoyloxy)propoxy]carbonylamino}(2S)-2-{[2-(4-fluorophenyl)phenyl]methyl}propanoic acid;
3-{[(1R)-2-methyl-1-(2-methylpropanoyloxy)propoxy]carbonylamino}(2S)-2-{[2-(4-fluorophenyl)phenyl]methyl}propanoic acid;
3-{[(1S)-2-methyl-1-(2-methylpropanoyloxy)propoxy]carbonylamino}(2S)-2-{[2-(4-fluorophenyl)phenyl]methyl}propanoic acid; and

a pharmaceutically acceptable salt of any of the foregoing.

Certain embodiments provide a compound of Formula (VI):

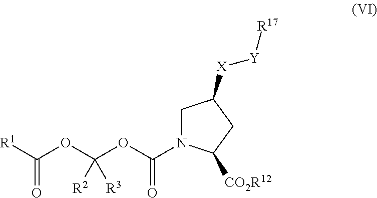

isomers thereof, and pharmaceutically acceptable salts of any of the foregoing, wherein:

X is selected from —O—, —S—, —NH—, and —CH₂—; and Y is selected from CH₂ and a bond; or X is selected from —CH₂—O—, —S—, —NH—, and —CH₂—; and Y is selected from —O—, —S—, —NH—, and —CH₂—;

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, heteroaryl, substituted heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₁₋₈ cycloalkyl, substituted C₁₋₈ cycloalkyl, C₃₋₈ heterocycloalkyl, and substituted C₃₋₈ heterocycloalkyl ring;

R¹² is selected from hydrogen and C₁₋₆ alkyl; and

R¹⁷ is selected from C₆₋₁₀ aryl, substituted C₆₋₁₀ aryl, C₃₋₈ cycloalkyl, substituted C₃₋₈ cycloalkyl, C₃₋₈ heterocycloalkyl, substituted C₃₋₈ heterocycloalkyl, C₅₋₁₀ heteroaryl, and substituted C₅₋₁₀ heteroaryl.

In certain embodiments of a compound of Formula (VI), X is selected from O and CH₂; Y is selected from a bond and CH₂; R¹² is hydrogen; and R¹⁷ is selected from phenyl, substituted phenyl wherein the one or more substituent groups is selected from halo and C₁₋₃ alkoxy, and cyclohexyl.

In certain embodiments of a compound of Formula (VI) the moiety:

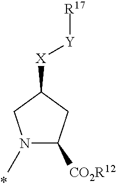

is derived from an alpha-2-delta ligand of the formula:

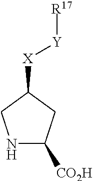

by replacing a hydrogen of the amine group with a covalent bond and by replacing the hydrogen atom of the carboxylic acid group with —R¹². In certain embodiments of a compound of Formula (VI), in the alpha-2-delta ligand of the above formula, X is selected from O and CH₂; Y is selected from a bond and CH₂; R¹² is hydrogen; and R¹⁷ is selected from phenyl, substituted phenyl wherein the one or more substituent groups is selected from halo and C₁₋₃ alkoxy, and cyclohexyl.

In certain embodiments of a compound of Formula (VI), the alpha-2-delta ligand is selected from:
(2S,4S)-4-[(3-chlorophenoxy)]pyrrolidine-2-carboxylic acid;
(2S,4S)-4-[(3-fluorophenyl)methyl]pyrrolidine-2-carboxylic acid;
(2S,4S)-4-[(2,3-difluorophenyl)methyl]pyrrolidine-2-carboxylic acid;
(2S,4S)-4-[(2,5-difluorophenyl)methyl]pyrrolidine-2-carboxylic acid;
(2S,4S)-4-[cyclohexylmethyl]pyrrolidine-2-carboxylic acid;
(2S,4S)-4-[(3-fluorophenoxy)methyl]-pyrrolidine-2-carboxylic acid;
(2S,4S)-4-[(2,3-difluorophenoxy)methyl]-pyrrolidine-2-carboxylic acid;
(2S,4S)-4-[(2,5-difluorophenoxy)methyl]-pyrrolidine-2-carboxylic acid;
(2S,4S)-4-[(3,6-difluorophenoxy)methyl]-pyrrolidine-2-carboxylic acid; and
(2S,4S)-4-[(3-methoxyphenoxy)methyl]-pyrrolidine-2-carboxylic acid.

In certain embodiments of a compound of Formula (VI), the compound is selected from:
1-{[(1R)-1-(1-acetyloxy)ethyl]oxycarbonyl}(2S,4S)-4-(3-chlorophenoxy)pyrrolidine-2-carboxylic acid;
1-{[(1S)-1-(1-acetyloxy)ethyl]oxycarbonyl}(2S,4S)-4-(3-chlorophenoxy)pyrrolidine-2-carboxylic acid;
1-{[(1R)-2-methyl-1-(1-acetyloxy)propyl]oxycarbonyl}(2S,4S)-4-(3-chlorophenoxy)pyrrolidine-2-carboxylic acid;
1-{[(1S)-2-methyl-1-(1-acetyloxy)propyl]oxycarbonyl}(2S,4S)-4-(3-chlorophenoxy)pyrrolidine-2-carboxylic acid;
1-{[(1R)-1-(1-propanoyloxy)ethyl]oxycarbonyl}(2S,4S)-4-(3-chlorophenoxy)pyrrolidine-2-carboxylic acid;
1-{[(1S)-1-(1-propanoyloxy)ethyl]oxycarbonyl}(2S,4S)-4-(3-chlorophenoxy)pyrrolidine-2-carboxylic acid;
1-{[(1R)-2-methyl-1-(1-propanoyloxy)propyl]oxycarbonyl}(2S,4S)-4-(3-chlorophenoxy)pyrrolidine-2-carboxylic acid;
1-{[(1S)-2-methyl-1-(1-propanoyloxy)propyl]oxycarbonyl}(2S,4S)-4-(3-chlorophenoxy)pyrrolidine-2-carboxylic acid;
1-{[(1R)-1-(2-methylpropanoyloxy)ethyl]oxycarbonyl}(2S,4S)-4-(3-chlorophenoxy)pyrrolidine-2-carboxylic acid;
1-{[(1S)-1-(2-methylpropanoyloxy)ethyl]oxycarbonyl}(2S,4S)-4-(3-chlorophenoxy)pyrrolidine-2-carboxylic acid;
1-{[(1R)-2-methyl-1-(2-methylpropanoyloxy)propyl]oxycarbonyl}(2S,4S)-4-(3-chlorophenoxy)pyrrolidine-2-carboxylic acid;
1-{[(1S)-2-methyl-1-(2-methylpropanoyloxy)propyl]oxycarbonyl}(2S,4S)-4-(3-chlorophenoxy)pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(3-fluorophenyl)methyl]pyrrolidine-2-carboxylic acid;
1-{[(1S)-1-(1-acetyloxy)ethyl]oxycarbonyl}(2S,4S)-4-[(3-fluorophenyl)methyl]pyrrolidine-2-carboxylic acid;
1-{[(1R)-2-methyl-1-(1-acetyloxy)propyl]oxycarbonyl}(2S,4S)-4-[(3-fluorophenyl)methyl]pyrrolidine-2-carboxylic acid;
1-{[(1S)-2-methyl-1-(1-acetyloxy)propyl]oxycarbonyl}(2S,4S)-4-[(3-fluorophenyl)methyl]pyrrolidine-2-carboxylic acid;
1-{[(1R)-1-(1-propanoyloxy)ethyl]oxycarbonyl}(2S,4S)-4-[(3-fluorophenyl)methyl]pyrrolidine-2-carboxylic acid;
1-{[(1S)-1-(1-propanoyloxy)ethyl]oxycarbonyl}(2S,4S)-4-[(3-fluorophenyl)methyl]pyrrolidine-2-carboxylic acid;
1-{[(1R)-2-methyl-1-(1-propanoyloxy)propyl]oxycarbonyl}(2S,4S)-4-[(3-fluorophenyl)methyl]pyrrolidine-2-carboxylic acid;
1-{[(1S)-2-methyl-1-(1-propanoyloxy)propyl]oxycarbonyl}(2S,4S)-4-[(3-fluorophenyl)methyl]pyrrolidine-2-carboxylic acid;
1-{[(1R)-1-(2-methylpropanoyloxy)ethyl]oxycarbonyl}(2S,4S)-4-[(3-fluorophenyl)methyl]pyrrolidine-2-carboxylic acid;
1-{[(1S)-1-(2-methylpropanoyloxy)ethyl]oxycarbonyl}(2S,4S)-4-[(3-fluorophenyl)methyl]pyrrolidine-2-carboxylic acid;
1-{[(1R)-2-methyl-1-(2-methylpropanoyloxy)propyl]oxycarbonyl}(2S,4S)-4-[(3-fluorophenyl)methyl]pyrrolidine-2-carboxylic acid;
1-{[(1S)-2-methyl-1-(2-methylpropanoyloxy)propyl]oxycarbonyl}(2S,4S)-4-[(3-fluorophenyl)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(2,3-difluorophenyl)methyl]pyrrolidine-2-carboxylic acid;
1-{[(1S)-1-(1-acetyloxy)ethyl]oxycarbonyl}(2S,4S)-4-[(2,3-difluorophenyl)methyl]pyrrolidine-2-carboxylic acid;
1-{[(1R)-2-methyl-1-(1-acetyloxy)propyl]oxycarbonyl}(2S,4S)-4-[(2,3-difluorophenyl)methyl]pyrrolidine-2-carboxylic acid;
1-{[(1S)-2-methyl-1-(1-acetyloxy)propyl]oxycarbonyl}(2S,4S)-4-[(2,3-difluorophenyl)methyl]pyrrolidine-2-carboxylic acid;
1-{[(1R)-1-(1-propanoyloxy)ethyl]oxycarbonyl}(2S,4S)-4-[(2,3-difluorophenyl)methyl]pyrrolidine-2-carboxylic acid;
1-{[(1S)-1-(1-propanoyloxy)ethyl]oxycarbonyl}(2S,4S)-4-[(2,3-difluorophenyl)methyl]pyrrolidine-2-carboxylic acid;
1-{[(1R)-2-methyl-1-(1-propanoyloxy)propyl]oxycarbonyl}(2S,4S)-4-[(2,3-difluorophenyl)methyl]pyrrolidine-2-carboxylic acid;
1-{[(1S)-2-methyl-1-(1-propanoyloxy)propyl]oxycarbonyl}(2S,4S)-4-[(2,3-difluorophenyl)methyl]pyrrolidine-2-carboxylic acid;
1-{[(1R)-1-(2-methylpropanoyloxy)ethyl]oxycarbonyl}(2S,4S)-4-[(2,3-difluorophenyl)methyl]pyrrolidine-2-carboxylic acid;
1-{[(1S)-1-(2-methylpropanoyloxy)ethyl]oxycarbonyl}(2S,4S)-4-[(2,3-difluorophenyl)methyl]pyrrolidine-2-carboxylic acid;
1-{[(1R)-2-methyl-1-(2-methylpropanoyloxy)propyl]oxycarbonyl}(2S,4S)-4-[(2,3-difluorophenyl)methyl]pyrrolidine-2-carboxylic acid;
1-{[(1S)-2-methyl-1-(2-methylpropanoyloxy)propyl]oxycarbonyl}(2S,4S)-4-[(2,3-difluorophenyl)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(2,5-difluorophenyl)methyl]pyrrolidine-2-carboxylic acid;
1-{[(1S)-1-(1-acetyloxy)ethyl]oxycarbonyl}(2S,4S)-4-[(2,5-difluorophenyl)methyl]pyrrolidine-2-carboxylic acid;
1-{[(1R)-2-methyl-1-(1-acetyloxy)propyl]oxycarbonyl}(2S,4S)-4-[(2,5-difluorophenyl)methyl]pyrrolidine-2-carboxylic acid;
1-{[(1S)-2-methyl-1-(1-acetyloxy)propyl]oxycarbonyl}(2S,4S)-4-[(2,5-difluorophenyl)methyl]pyrrolidine-2-carboxylic acid;
1-{[(1R)-1-(1-propanoyloxy)ethyl]oxycarbonyl}(2S,4S)-4-[(2,5-difluorophenyl)methyl]pyrrolidine-2-carboxylic acid;
1-{[(1S)-1-(1-propanoyloxy)ethyl]oxycarbonyl}(2S,4S)-4-[(2,5-difluorophenyl)methyl]pyrrolidine-2-carboxylic acid;
1-{[(1R)-2-methyl-1-(1-propanoyloxy)propyl]oxycarbonyl}(2S,4S)-4-[(2,5-difluorophenyl)methyl]pyrrolidine-2-carboxylic acid;
1-{[(1S)-2-methyl-1-(1-propanoyloxy)propyl]oxycarbonyl}(2S,4S)-4-[(2,5-difluorophenyl)methyl]pyrrolidine-2-carboxylic acid;
1-{[(1R)-1-(2-methylpropanoyloxy)ethyl]oxycarbonyl}(2S,4S)-4-[(2,5-difluorophenyl)methyl]pyrrolidine-2-carboxylic acid;
1-{[(1S)-1-(2-methylpropanoyloxy)ethyl]oxycarbonyl}(2S,4S)-4-[(2,5-difluorophenyl)methyl]pyrrolidine-2-carboxylic acid;
1-{[(1R)-2-methyl-1-(2-methylpropanoyloxy)propyl]oxycarbonyl}(2S,4S)-4-[(2,5-difluorophenyl)methyl]pyrrolidine-2-carboxylic acid;
1-{[(1S)-2-methyl-1-(2-methylpropanoyloxy)propyl]oxycarbonyl}(2S,4S)-4-[(2,5-difluorophenyl)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-(cyclohexylmethyl)pyrrolidine-2-carboxylic acid;
1-{[(1S)-1-(1-acetyloxy)ethyl]oxycarbonyl}(2S,4S)-4-(cyclohexylmethyl)pyrrolidine-2-carboxylic acid;
1-{[(1R)-2-methyl-1-(1-acetyloxy)propyl]oxycarbonyl}(2S,4S)-4-(cyclohexylmethyl)pyrrolidine-2-carboxylic acid;
1-{[(1S)-2-methyl-1-(1-acetyloxy)propyl]oxycarbonyl}(2S,4S)-4-(cyclohexylmethyl)pyrrolidine-2-carboxylic acid;
1-{[(1R)-1-(1-propanoyloxy)ethyl]oxycarbonyl}(2S,4S)-4-(cyclohexylmethyl)pyrrolidine-2-carboxylic acid;
1-{[(1S)-1-(1-propanoyloxy)ethyl]oxycarbonyl}(2S,4S)-4-(cyclohexylmethyl)pyrrolidine-2-carboxylic acid;
1-{[(1R)-2-methyl-1-(1-propanoyloxy)propyl]oxycarbonyl}(2S,4S)-4-(cyclohexylmethyl)pyrrolidine-2-carboxylic acid;
1-{[(1S)-2-methyl-1-(1-propanoyloxy)propyl]oxycarbonyl}(2S,4S)-4-(cyclohexylmethyl)pyrrolidine-2-carboxylic acid;
1-{[(1R)-1-(2-methylpropanoyloxy)ethyl]oxycarbonyl}(2S,4S)-4-(cyclohexylmethyl)pyrrolidine-2-carboxylic acid;
1-{[(1S)-1-(2-methylpropanoyloxy)ethyl]oxycarbonyl}(2S,4S)-4-(cyclohexylmethyl)pyrrolidine-2-carboxylic acid;
1-{[(1R)-2-methyl-1-(2-methylpropanoyloxy)propyl]oxycarbonyl}(2S,4S)-4-(cyclohexylmethyl)pyrrolidine-2-carboxylic acid;
1-{[(1S)-2-methyl-1-(2-methylpropanoyloxy)propyl]oxycarbonyl}(2S,4S)-4-(cyclohexylmethyl)pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(3-fluorophenoxy)methyl]pyrrolidine-2-carboxylic acid;
1-{[(1S)-1-(1-acetyloxy)ethyl]oxycarbonyl}(2S,4S)-4-[(3-fluorophenoxy)methyl]pyrrolidine-2-carboxylic acid;
1-{[(1R)-2-methyl-1-(1-acetyloxy)propyl]oxycarbonyl}(2S,4S)-4-[(3-fluorophenoxy)methyl]pyrrolidine-2-carboxylic acid;
1-{[(1S)-2-methyl-1-(1-acetyloxy)propyl]oxycarbonyl}(2S,4S)-4-[(3-fluorophenoxy)methyl]pyrrolidine-2-carboxylic acid;
1-{[(1R)-1-(1-propanoyloxy)ethyl]oxycarbonyl}(2S,4S)-4-[(3-fluorophenoxy)methyl]pyrrolidine-2-carboxylic acid;
1-{[(1S)-1-(1-propanoyloxy)ethyl]oxycarbonyl}(2S,4S)-4-[(3-fluorophenoxy)methyl]pyrrolidine-2-carboxylic acid;
1-{[(1R)-2-methyl-1-(1-propanoyloxy)propyl]oxycarbonyl}(2S,4S)-4-[(3-fluorophenoxy)methyl]pyrrolidine-2-carboxylic acid;
1-{[(1S)-2-methyl-1-(1-propanoyloxy)propyl]oxycarbonyl}(2S,4S)-4-[(3-fluorophenoxy)methyl]pyrrolidine-2-carboxylic acid;
1-{[(1R)-1-(2-methylpropanoyloxy)ethyl]oxycarbonyl}(2S,4S)-4-[(3-fluorophenoxy)methyl]pyrrolidine-2-carboxylic acid;
1-{[(1S)-1-(2-methylpropanoyloxy)ethyl]oxycarbonyl}(2S,4S)-4-[(3-fluorophenoxy)methyl]pyrrolidine-2-carboxylic acid;
1-{[(1R)-2-methyl-1-(2-methylpropanoyloxy)propyl]oxycarbonyl}(2S,4S)-4-[(3-fluorophenoxy)methyl]pyrrolidine-2-carboxylic acid;
1-{[(1S)-2-methyl-1-(2-methylpropanoyloxy)propyl]oxycarbonyl}(2S,4S)-4-[(3-fluorophenoxy)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(2,3-difluorophenoxy)methyl]pyrrolidine-2-carboxylic acid;
1-{[(1S)-1-(1-acetyloxy)ethyl]oxycarbonyl}(2S,4S)-4-[(2,3-difluorophenoxy)methyl]pyrrolidine-2-carboxylic acid;
1-{[(1R)-2-methyl-1-(1-acetyloxy)propyl]oxycarbonyl}(2S,4S)-4-[(2,3-difluorophenoxy)methyl]pyrrolidine-2-carboxylic acid;
1-{[(1S)-2-methyl-1-(1-acetyloxy)propyl]oxycarbonyl}(2S,4S)-4-[(2,3-difluorophenoxy)methyl]pyrrolidine-2-carboxylic acid;
1-{[(1R)-1-(1-propanoyloxy)ethyl]oxycarbonyl}(2S,4S)-4-[(2,3-difluorophenoxy)methyl]pyrrolidine-2-carboxylic acid;
1-{[(1S)-1-(1-propanoyloxy)ethyl]oxycarbonyl}(2S,4S)-4-[(2,3-difluorophenoxy)methyl]pyrrolidine-2-carboxylic acid;
1-{[(1R)-2-methyl-1-(1-propanoyloxy)propyl]oxycarbonyl}(2S,4S)-4-[(2,3-difluorophenoxy)methyl]pyrrolidine-2-carboxylic acid;
1-{[(1S)-2-methyl-1-(1-propanoyloxy)propyl]oxycarbonyl}(2S,4S)-4-[(2,3-difluorophenoxy)methyl]pyrrolidine-2-carboxylic acid;
1-{[(1R)-1-(2-methylpropanoyloxy)ethyl]oxycarbonyl}(2S,4S)-4-[(2,3-difluorophenoxy)methyl]pyrrolidine-2-carboxylic acid;
1-{[(1S)-1-(2-methylpropanoyloxy)ethyl]oxycarbonyl}(2S,4S)-4-[(2,3-difluorophenoxy)methyl]pyrrolidine-2-carboxylic acid;
1-{[(1R)-2-methyl-1-(2-methylpropanoyloxy)propyl]oxycarbonyl}(2S,4S)-4-[(2,3-difluorophenoxy)methyl]pyrrolidine-2-carboxylic acid;
1-{[(1S)-2-methyl-1-(2-methylpropanoyloxy)propyl]oxycarbonyl}(2S,4S)-4-[(2,3-difluorophenoxy)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(2,5-difluorophenoxy)methyl]pyrrolidine-2-carboxylic acid;
1-{[(1S)-1-(1-acetyloxy)ethyl]oxycarbonyl}(2S,4S)-4-[(2,5-difluorophenoxy)methyl]pyrrolidine-2-carboxylic acid;
1-{[(1R)-2-methyl-1-(1-acetyloxy)propyl]oxycarbonyl}(2S,4S)-4-[(2,5-difluorophenoxy)methyl]pyrrolidine-2-carboxylic acid;
1-{[(1S)-2-methyl-1-(1-acetyloxy)propyl]oxycarbonyl}(2S,4S)-4-[(2,5-difluorophenoxy)methyl]pyrrolidine-2-carboxylic acid;
1-{[(1R)-1-(1-propanoyloxy)ethyl]oxycarbonyl}(2S,4S)-4-[(2,5-difluorophenoxy)methyl]pyrrolidine-2-carboxylic acid;
1-{[(1S)-1-(1-propanoyloxy)ethyl]oxycarbonyl}(2S,4S)-4-[(2,5-difluorophenoxy)methyl]pyrrolidine-2-carboxylic acid;
1-{[(1R)-2-methyl-1-(1-propanoyloxy)propyl]oxycarbonyl}(2S,4S)-4-[(2,5-difluorophenoxy)methyl]pyrrolidine-2-carboxylic acid;
1-{[(1S)-2-methyl-1-(1-propanoyloxy)propyl]oxycarbonyl}(2S,4S)-4-[(2,5-difluorophenoxy)methyl]pyrrolidine-2-carboxylic acid;
1-{[(1R)-1-(2-methylpropanoyloxy)ethyl]oxycarbonyl}(2S,4S)-4-[(2,5-difluorophenoxy)methyl]pyrrolidine-2-carboxylic acid;
1-{[(1S)-1-(2-methylpropanoyloxy)ethyl]oxycarbonyl}(2S,4S)-4-[(2,5-difluorophenoxy)methyl]pyrrolidine-2-carboxylic acid;
1-{[(1R)-2-methyl-1-(2-methylpropanoyloxy)propyl]oxycarbonyl}(2S,4S)-4-[(2,5-difluorophenoxy)methyl]pyrrolidine-2-carboxylic acid;
1-{[(1S)-2-methyl-1-(2-methylpropanoyloxy)propyl]oxycarbonyl}(2S,4S)-4-[(2,5-difluorophenoxy)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(3,6-difluorophenoxy)methyl]pyrrolidine-2-carboxylic acid;
1-{[(1S)-1-(1-acetyloxy)ethyl]oxycarbonyl}(2S,4S)-4-[(3,6-difluorophenoxy)methyl]pyrrolidine-2-carboxylic acid;
1-{[(1R)-2-methyl-1-(1-acetyloxy)propyl]oxycarbonyl}(2S,4S)-4-[(3,6-difluorophenoxy)methyl]pyrrolidine-2-carboxylic acid;
1-{[(1S)-2-methyl-1-(1-acetyloxy)propyl]oxycarbonyl}(2S,4S)-4-[(3,6-difluorophenoxy)methyl]pyrrolidine-2-carboxylic acid;
1-{[(1R)-1-(1-propanoyloxy)ethyl]oxycarbonyl}(2S,4S)-4-[(3,6-difluorophenoxy)methyl]pyrrolidine-2-carboxylic acid;
1-{[(1S)-1-(1-propanoyloxy)ethyl]oxycarbonyl}(2S,4S)-4-[(3,6-difluorophenoxy)methyl]pyrrolidine-2-carboxylic acid;
1-{[(1R)-2-methyl-1-(1-propanoyloxy)propyl]oxycarbonyl}(2S,4S)-4-[(3,6-difluorophenoxy)methyl]pyrrolidine-2-carboxylic acid;
1-{[(1S)-2-methyl-1-(1-propanoyloxy)propyl]oxycarbonyl}(2S,4S)-4-[(3,6-difluorophenoxy)methyl]pyrrolidine-2-carboxylic acid;
1-{[(1R)-1-(2-methylpropanoyloxy)ethyl]oxycarbonyl}(2S,4S)-4-[(3,6-difluorophenoxy)methyl]pyrrolidine-2-carboxylic acid;
1-{[(1S)-1-(2-methylpropanoyloxy)ethyl]oxycarbonyl}(2S,4S)-4-[(3,6-difluorophenoxy)methyl]pyrrolidine-2-carboxylic acid;
1-{[(1R)-2-methyl-1-(2-methylpropanoyloxy)propyl]oxycarbonyl}(2S,4S)-4-[(3,6-difluorophenoxy)methyl]pyrrolidine-2-carboxylic acid;
1-{[(1S)-2-methyl-1-(2-methylpropanoyloxy)propyl]oxycarbonyl}(2S,4S)-4-[(3,6-difluorophenoxy)methyl]pyrrolidine-2-carboxylic acid; and

a pharmaceutically acceptable salt of any of the foregoing.

In certain embodiments of a compound of Formula (VI), the compound is selected from:
1-{[(1R)-1-(1-acetyloxy)ethyl]oxycarbonyl}(2S,4S)-4-[(3-methoxyphenoxy)methyl]pyrrolidine-2-carboxylic acid;
1-{[(1S)-1-(1-acetyloxy)ethyl]oxycarbonyl}(2S,4S)-4-[(3-methoxyphenoxy)methyl]pyrrolidine-2-carboxylic acid;
1-{[(1R)-2-methyl-1-(1-acetyloxy)propyl]oxycarbonyl}(2S,4S)-4-[(3-methoxyphenoxy)methyl]pyrrolidine-2-carboxylic acid;
1-{[(1S)-2-methyl-1-(1-acetyloxy)propyl]oxycarbonyl}(2S,4S)-4-[(3-methoxyphenoxy)methyl]pyrrolidine-2-carboxylic acid;
1-{[(1R)-1-(1-propanoyloxy)ethyl]oxycarbonyl}(2S,4S)-4-[(3-methoxyphenoxy)methyl]pyrrolidine-2-carboxylic acid;
1-{[(1S)-1-(1-propanoyloxy)ethyl]oxycarbonyl}(2S,4S)-4-[(3-methoxyphenoxy)methyl]pyrrolidine-2-carboxylic acid;
1-{[(1R)-2-methyl-1-(1-propanoyloxy)propyl]oxycarbonyl}(2S,4S)-4-[(3-methoxyphenoxy)methyl]pyrrolidine-2-carboxylic acid;
1-{[(1S)-2-methyl-1-(1-propanoyloxy)propyl]oxycarbonyl}(2S,4S)-4-[(3-methoxyphenoxy)methyl]pyrrolidine-2-carboxylic acid;
1-{[(1R)-1-(2-methylpropanoyloxy)ethyl]oxycarbonyl}(2S,4S)-4-[(3-methoxyphenoxy)methyl]pyrrolidine-2-carboxylic acid;
1-{[(1S)-1-(2-methylpropanoyloxy)ethyl]oxycarbonyl}(2S,4S)-4-[(3-methoxyphenoxy)methyl]pyrrolidine-2-carboxylic acid;
1-{[(1R)-2-methyl-1-(2-methylpropanoyloxy)propyl]oxycarbonyl}(2S,4S)-4-[(3-methoxyphenoxy)methyl]pyrrolidine-2-carboxylic acid;
1-{[(1S)-2-methyl-1-(2-methylpropanoyloxy)propyl]oxycarbonyl}(2S,4S)-4-[(3-methoxyphenoxy)methyl]pyrrolidine-2-carboxylic acid; and

a pharmaceutically acceptable salt of any of the foregoing.

Certain embodiments provide a compound of Formula (VII):

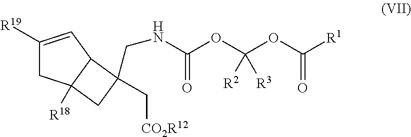

isomers thereof, and pharmaceutically acceptable salts of any of the foregoing, wherein:

R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;

R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and substituted C₃₋₈ heterocycloalkyl ring;

R¹² is selected from hydrogen and C₁₋₆ alkyl; and

R¹⁸ and R¹⁹ are independently selected from hydrogen and C₁₋₆ alkyl.

In certain embodiments of a compound of Formula (VII), R¹² is hydrogen; and R¹⁸ and R¹⁹ are independently selected from hydrogen, methyl, and ethyl.

In certain embodiments of a compound of Formula (VII) the moiety:

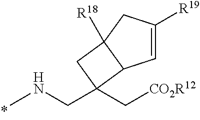

is derived from an alpha-2-delta ligand of the formula:

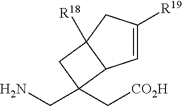

by replacing a hydrogen of the amine group with a covalent bond and by replacing the hydrogen atom of the carboxylic acid group with —R¹². In certain embodiments of a compound of Formula (VII), in the alpha-2-delta ligand of the above formula, R¹² is hydrogen; and R¹⁸ and R¹⁹ are independently selected from hydrogen, methyl, and ethyl.

In certain embodiments of a compound of Formula (VII), the alpha-2-delta ligand is selected from:
(1S,5R,6R)-6-(aminomethyl)-bicyclo[3.2.0]hept-3-ene-6-acetic acid;
(1R,5S,6S)-6-(aminomethyl)-3-methyl-bicyclo[3.2.0]hept-3-ene-6-acetic acid;
(1R,5S,6S)-6-(aminomethyl)-3-ethyl-bicyclo[3.2.0]hept-3-ene-6-acetic acid;
(1S,5R,6R)-6-(aminomethyl)-3-methyl-bicyclo[3.2.0]hept-3-ene-6-acetic acid;
(1S,5R,6R)-6-(aminomethyl)-3-ethyl-bicyclo[3.2.0]hept-3-ene-6-acetic acid; and
(1S,5R,6R)-6-(aminomethyl)-1-ethyl-bicyclo[3.2.0]hept-3-ene-6-acetic acid.

In certain embodiments of a compound of Formula (VII), the compound is selected from:
2-(6-{[(1R)-1-(1-acetyloxyethoxy)carbonylamino]methyl}(1S,5R,6R)-bicyclo[3.2.0]hept-3-en-6-yl)acetic acid;
2-(6-{[(1S)-1-(1-acetyloxyethoxy)carbonylamino]methyl}(1S,5R,6R)-bicyclo[3.2.0]hept-3-en-6-yl)acetic acid;
2-(6-{[((1R)-1-(1-propanoyloxy)ethoxy)carbonylamino]methyl}(1S,5R,6R)-bicyclo[3.2.0]hept-3-en-6-yl)acetic acid;
2-(6-{[((1S)-1-(1-propanoyloxy)ethoxy)carbonylamino]methyl}(1S,5R,6R)-bicyclo[3.2.0]hept-3-en-6-yl)acetic acid;
2-[6-({[(1R)-1-(2-methylpropanoyloxy)ethoxy]carbonylamino}methyl)(1S,5R,6R)-bicyclo[3.2.0]hept-3-en-6-yl]acetic acid;
2-[6-({[(1S)-1-(2-methylpropanoyloxy)ethoxy]carbonylamino}methyl)(1S,5R,6R)-bicyclo[3.2.0]hept-3-en-6-yl]acetic acid;
2-[6-{[((1R)-1-acetyloxy-2-methylpropoxy)carbonylamino]methyl}(1S,5R,6R)-bicyclo[3.2.0]hept-3-en-6-yl]acetic acid;
2-[6-{[((1S)-1-acetyloxy-2-methylpropoxy)carbonylamino]methyl}(1S,5R,6R)-bicyclo[3.2.0]hept-3-en-6-yl]acetic acid;
2-[6-{[((1R)-2-methyl-1-propanoyloxypropoxy)carbonylamino]methyl}(1S,5R,6R)-bicyclo[3.2.0]hept-3-en-6-yl]acetic acid;
2-[6-{[((1S)-2-methyl-1-propanoyloxypropoxy)carbonylamino]methyl}(1S,5R,6R)-bicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-bicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-bicyclo[3.2.0]hept-3-en-6-yl]acetic acid; and

a pharmaceutically acceptable salt of any of the foregoing.

In certain embodiments of a compound of Formula (VII), the compound is selected from:
2-(6-{[(1R)-1-(1-acetyloxyethoxy)carbonylamino]methyl}(1R,5S,6S)-3-methylbicyclo[3.2.0]hept-3-en-6-yl)acetic acid;
2-(6-{[(1S)-1-(1-acetyloxyethoxy)carbonylamino]methyl}(1R,5S,6S)-3-methylbicyclo[3.2.0]hept-3-en-6-yl)acetic acid;
2-(6-{[((1R)-1-(1-propanoyloxy)ethoxy)carbonylamino]methyl}(1R,5S,6S)-3-methylbicyclo[3.2.0]hept-3-en-6-yl)acetic acid;
2-(6-{[((1S)-1-(1-propanoyloxy)ethoxy)carbonylamino]methyl}(1R,5S,6S)-3-methylbicyclo[3.2.0]hept-3-en-6-yl)acetic acid;
2-[6-({[(1R)-1-(2-methylpropanoyloxy)ethoxy]carbonylamino}methyl)(1R,5S,6S)-3-methylbicyclo[3.2.0]hept-3-en-6-yl]acetic acid;
2-[6-({[(1S)-1-(2-methylpropanoyloxy)ethoxy]carbonylamino}methyl)(1R,5S,6S)-3-methylbicyclo[3.2.0]hept-3-en-6-yl]acetic acid;
2-[6-{[((1R)-1-acetyloxy-2-methylpropoxy)carbonylamino]methyl}(1R,5S,6S)-3-methylbicyclo[3.2.0]hept-3-en-6-yl]acetic acid;
2-[6-{[((1S)-1-acetyloxy-2-methylpropoxy)carbonylamino]methyl}(1R,5S,6S)-3-methylbicyclo[3.2.0]hept-3-en-6-yl]acetic acid;
2-[6-{[((1R)-2-methyl-1-propanoyloxypropoxy)carbonylamino]methyl}(1R,5S,6S)-3-methylbicyclo[3.2.0]hept-3-en-6-yl]acetic acid;
2-[6-{[((1S)-2-methyl-1-propanoyloxypropoxy)carbonylamino]methyl}(1R,5S,6S)-3-methylbicyclo[3.2.0]hept-3-en-6-yl]acetic acid;
2-[6-({[(1R)-2-methyl-1-(2-methylpropanoyloxy)propoxy]carbonylamino}methyl)(1R,5S,6S)-3-methylbicyclo[3.2.0]hept-3-en-6-yl]acetic acid;
2-[6-({[(1S)-2-methyl-1-(2-methylpropanoyloxy)propoxy]carbonylamino}methyl)(1R,5S,6S)-3-methylbicyclo[3.2.0]hept-3-en-6-yl]acetic acid; and

a pharmaceutically acceptable salt of any of the foregoing.

In certain embodiments of a compound of Formula (VII), the compound is selected from:
2-(6-{[(1R)-1-(1-acetyloxyethoxy)carbonylamino]methyl}(1R,5S,6S)-3-ethylbicyclo[3.2.0]hept-3-en-6-yl)acetic acid;
2-(6-{[(1S)-1-(1-acetyloxyethoxy)carbonylamino]methyl}(1R,5S,6S)-3-ethylbicyclo[3.2.0]hept-3-en-6-yl)acetic acid;
2-(6-{[((1R)-1-(1-propanoyloxy)ethoxy)carbonylamino]methyl}(1R,5S,6S)-3-ethylbicyclo[3.2.0]hept-3-en-6-yl)acetic acid;
2-(6-{[((1S)-1-(1-propanoyloxy)ethoxy)carbonylamino]methyl}(1R,5S,6S)-3-ethylbicyclo[3.2.0]hept-3-en-6-yl)acetic acid;
2-[6-({[(1R)-1-(2-ethylpropanoyloxy)ethoxy]carbonylamino}methyl)(1R,5S,6S)-3-ethylbicyclo[3.2.0]hept-3-en-6-yl]acetic acid;
2-[6-({[(1S)-1-(2-ethylpropanoyloxy)ethoxy]carbonylamino}methyl)(1R,5S,6S)-3-ethylbicyclo[3.2.0]hept-3-en-6-yl]acetic acid;
2-[6-{[((1R)-1-acetyloxy-2-methylpropoxy)carbonylamino]methyl}(1R,5S,6S)-3-ethylbicyclo[3.2.0]hept-3-en-6-yl]acetic acid;
2-[6-{[((1S)-1-acetyloxy-2-methylpropoxy)carbonylamino]methyl}(1R,5S,6S)-3-ethylbicyclo[3.2.0]hept-3-en-6-yl]acetic acid;
2-[6-{[((1R)-2-methyl-1-propanoyloxypropoxy)carbonylamino]methyl}(1R,5S,6S)-3-ethylbicyclo[3.2.0]hept-3-en-6-yl]acetic acid;
2-[6-{[((1S)-2-methyl-1-propanoyloxypropoxy)carbonylamino]methyl}(1R,5S,6S)-3-ethylbicyclo[3.2.0]hept-3-en-6-yl]acetic acid;
2-[6-({[(1R)-2-methyl-1-(2-methylpropanoyloxy)propoxy]carbonylamino}methyl)(1R,5S,6S)-3-ethylbicyclo[3.2.0]hept-3-en-6-yl]acetic acid;
2-[6-({[(1S)-2-methyl-1-(2-methylpropanoyloxy)propoxy]carbonylamino}methyl)(1R,5S,6S)-3-ethylbicyclo[3.2.0]hept-3-en-6-yl]acetic acid; and

a pharmaceutically acceptable salt of any of the foregoing.

In certain embodiments of a compound of Formula (VII), the compound is selected from:
2-(6-{[(1R)-1-(1-acetyloxyethoxy)carbonylamino]methyl}(1S,5R,6R)-3-methylbicyclo[3.2.0]hept-3-en-6-yl)acetic acid;
2-(6-{[(1S)-1-(1-acetyloxyethoxy)carbonylamino]methyl}(1S,5R,6R)-3-methylbicyclo[3.2.0]hept-3-en-6-yl)acetic acid;
2-(6-{[((1R)-1-(1-propanoyloxy)ethoxy)carbonylamino]methyl}(1S,5R,6R)-3-methylbicyclo[3.2.0]hept-3-en-6-yl)acetic acid;
2-(6-{[((1S)-1-(1-propanoyloxy)ethoxy)carbonylamino]methyl}(1S,5R,6R)-3-methylbicyclo[3.2.0]hept-3-en-6-yl)acetic acid;
2-[6-({[(1R)-1-(2-methylpropanoyloxy)ethoxy]carbonylamino}methyl)(1S,5R,6R)-3-methylbicyclo[3.2.0]hept-3-en-6-yl]acetic acid;
2-[6-({[(1S)-1-(2-methylpropanoyloxy)ethoxy]carbonylamino}methyl)(1S,5R,6R)-3-methylbicyclo[3.2.0]hept-3-en-6-yl]acetic acid;
2-[6-{[((1R)-1-acetyloxy-2-methylpropoxy)carbonylamino]methyl}(1S,5R,6R)-3-methylbicyclo[3.2.0]hept-3-en-6-yl]acetic acid;
2-[6-{[((1S)-1-acetyloxy-2-methylpropoxy)carbonylamino]methyl}(1S,5R,6R)-3-methylbicyclo[3.2.0]hept-3-en-6-yl]acetic acid;
2-[6-{[((1R)-2-methyl-1-propanoyloxypropoxy)carbonylamino]methyl}(1S,5R,6R)-3-methylbicyclo[3.2.0]hept-3-en-6-yl]acetic acid;
2-[6-{[((1S)-2-methyl-1-propanoyloxypropoxy)carbonylamino]methyl}(1S,5R,6R)-3-methylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-3-methylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-3-methylbicyclo[3.2.0]hept-3-en-6-yl]acetic acid; and

a pharmaceutically acceptable salt of any of the foregoing.

In certain embodiments of a compound of Formula (VII), the compound is selected from:
2-(6-{[(1R)-1-(1-acetyloxyethoxy)carbonylamino]methyl}(1S,5R,6R)-3-ethylbicyclo[3.2.0]hept-3-en-6-yl)acetic acid;
2-(6-{[(1S)-1-(1-acetyloxyethoxy)carbonylamino]methyl}(1S,5R,6R)-3-ethylbicyclo[3.2.0]hept-3-en-6-yl)acetic acid;
2-(6-{[((1R)-1-(1-propanoyloxy)ethoxy)carbonylamino]methyl}(1S,5R,6R)-3-ethylbicyclo[3.2.0]hept-3-en-6-yl)acetic acid;
2-(6-{[((1S)-1-(1-propanoyloxy)ethoxy)carbonylamino]methyl}(1S,5R,6R)-3-ethylbicyclo[3.2.0]hept-3-en-6-yl)acetic acid;
2-[6-({[(1R)-1-(2-methylpropanoyloxy)ethoxy]carbonylamino}methyl)(1S,5R,6R)-3-ethylbicyclo[3.2.0]hept-3-en-6-yl]acetic acid;
2-[6-({[(1S)-1-(2-methylpropanoyloxy)ethoxy]carbonylamino}methyl)(1S,5R,6R)-3-ethylbicyclo[3.2.0]hept-3-en-6-yl]acetic acid;
2-[6-{[((1R)-1-acetyloxy-2-methylpropoxy)carbonylamino]methyl}(1S,5R,6R)-3-ethylbicyclo[3.2.0]hept-3-en-6-yl]acetic acid;
2-[6-{[((1S)-1-acetyloxy-2-methylpropoxy)carbonylamino]methyl}(1S,5R,6R)-3-ethylbicyclo[3.2.0]hept-3-en-6-yl]acetic acid;
2-[6-{[((1R)-2-methyl-1-propanoyloxypropoxy)carbonylamino]methyl}(1S,5R,6R)-3-ethylbicyclo[3.2.0]hept-3-en-6-yl]acetic acid;
2-[6-{[((1S)-2-methyl-1-propanoyloxypropoxy)carbonylamino]methyl}(1S,5R,6R)-3-ethylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-3-ethylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-3-ethylbicyclo[3.2.0]hept-3-en-6-yl]acetic acid; and

a pharmaceutically acceptable salt of any of the foregoing.

In certain embodiments of a compound of Formula (VII), the compound is selected from:
2-(6-{[(1R)-1-(1-acetyloxyethoxy)carbonylamino]methyl}(1S,5R,6R)-1-ethylbicyclo[3.2.0]hept-3-en-6-yl)acetic acid;
2-(6-{[(1S)-1-(1-acetyloxyethoxy)carbonylamino]methyl}(1S,5R,6R)-1-ethylbicyclo[3.2.0]hept-3-en-6-yl)acetic acid;
2-(6-{[((1R)-1-(1-propanoyloxy)ethoxy)carbonylamino]methyl}(1S,5R,6R)-1-ethylbicyclo[3.2.0]hept-3-en-6-yl)acetic acid;
2-(6-{[((1S)-1-(1-propanoyloxy)ethoxy)carbonylamino]methyl}(1S,5R,6R)-1-ethylbicyclo[3.2.0]hept-3-en-6-yl)acetic acid;
2-[6-({[(1R)-1-(2-methylpropanoyloxy)ethoxy]carbonylamino}methyl)(1S,5R,6R)-1-ethylbicyclo[3.2.0]hept-3-en-6-yl]acetic acid;
2-[6-({[(1S)-1-(2-methylpropanoyloxy)ethoxy]carbonylamino}methyl)(1S,5R,6R)-1-ethylbicyclo[3.2.0]hept-3-en-6-yl]acetic acid;
2-[6-{[((1R)-1-acetyloxy-2-methylpropoxy)carbonylamino]methyl}(1S,5R,6R)-1-ethylbicyclo[3.2.0]hept-3-en-6-yl]acetic acid;
2-[6-{[((1S)-1-acetyloxy-2-methylpropoxy)carbonylamino]methyl}(1S,5R,6R)-1-ethylbicyclo[3.2.0]hept-3-en-6-yl]acetic acid;
2-[6-{[((1R)-2-methyl-1-propanoyloxypropoxy)carbonylamino]methyl}(1S,5R,6R)-1-ethylbicyclo[3.2.0]hept-3-en-6-yl]acetic acid;
2-[6-{[((1S)-2-methyl-1-propanoyloxypropoxy)carbonylamino]methyl}(1S,5R,6R)-1-ethylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-1-ethylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-1-ethylbicyclo[3.2.0]hept-3-en-6-yl]acetic acid; and

a pharmaceutically acceptable salt of any of the foregoing.

In certain embodiments of compounds of Formulae (I)-(VII), R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₅₋₁₀ heteroaryl, and substituted C₅₋₁₀ heteroaryl. In certain embodiments of compounds of Formulae (I)-(VII), R¹ is selected from methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl, and 3-pyridyl. In certain embodiments of compounds of Formulae (I)-(VII), R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl.

In certain embodiments of compounds of Formulae (I)-(VII), R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, C₅₋₁₀ heteroaryl, and substituted C₅₋₁₀ heteroaryl. In certain embodiments of compounds of Formulae (I)-(VII), R² and R³ are independently selected from hydrogen, methyl, ethyl, propyl, isopropyl, sec-butyl, tent-butyl, cyclopentyl, cyclohexyl, phenyl, benzyl, phenethyl, 3-pyridyl, methoxycarbonyl, ethoxycarbonyl, propoxycarbonyl, isopropoxycarbonyl, butoxycarbonyl, isobutoxycarbonyl, sec-butoxycarbonyl, tent-butoxycarbonyl, and cyclohexyloxycarbonyl. In certain embodiments of compounds of Formulae (I)-(VII), R² is hydrogen; and R³ is selected from hydrogen, methyl, ethyl, propyl, and isopropyl.

In certain embodiments of compounds of Formula (I), each of R² and R³ is hydrogen; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formula (I), each of R² and R³ is hydrogen; and R¹ is selected from methyl, ethyl, propyl, isopropyl, phenyl, benzyl, phenethyl, cyclopentyl, cyclohexyl, and 3-pyridyl. In certain embodiments of compounds of Formula (I), each of R² and R³ is hydrogen; and R¹ is selected from methyl, ethyl, propyl, and isopropyl.

In certain embodiments of compounds of Formula (I), each of R² and R³ is methyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formula (I), each of R² and R³ is methyl; and R¹ is selected from methyl, ethyl, propyl, isopropyl, phenyl, benzyl, phenethyl, cyclopentyl, cyclohexyl, and 3-pyridyl. In certain embodiments of compounds of Formula (I), each of R² and R³ is methyl; and R¹ is selected from methyl, ethyl, propyl, and isopropyl.

In certain embodiments of compounds of Formula (I), each of R² and R³ is ethyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formula (I), each of R² and R³ is ethyl; and R¹ is selected from methyl, ethyl, propyl, isopropyl, phenyl, benzyl, phenethyl, cyclopentyl, cyclohexyl, and 3-pyridyl. In certain embodiments of compounds of Formula (I), each of R² and R³ is ethyl; and R¹ is selected from methyl, ethyl, propyl, and isopropyl.

In certain embodiments of compounds of Formula (I), each of R² and R³ is propyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formula (I), each of R² and R³ is propyl; and R¹ is selected from methyl, ethyl, propyl, isopropyl, phenyl, benzyl, phenethyl, cyclopentyl, cyclohexyl, and 3-pyridyl. In certain embodiments of compounds of Formula (I), each of R² and R³ is propyl; and R¹ is selected from methyl, ethyl, propyl, and isopropyl.

In certain embodiments of compounds of Formula (I), each of R² and R³ is isopropyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formula (I), each of R² and R³ is isopropyl; and R¹ is selected from methyl, ethyl, propyl, isopropyl, phenyl, benzyl, phenethyl, cyclopentyl, cyclohexyl, and 3-pyridyl. In certain embodiments of compounds of Formula (I), each of R² and R³ is isobutyl; and R¹ is selected from methyl, ethyl, propyl, and isopropyl.

In certain embodiments of compounds of Formulae (II)-(VII), each of R² and R¹² is hydrogen; R³ is hydrogen; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formulae (II)-(VII), each of R² and R¹² is hydrogen; R³ is hydrogen; and R¹ is selected from methyl, ethyl, propyl, isopropyl, phenyl, benzyl, phenethyl, cyclopentyl, cyclohexyl, and 3-pyridyl. In certain embodiments of compounds of Formulae (II)-(VII), each of R² and R¹² is hydrogen; R³ is hydrogen; and R¹ is selected from methyl, ethyl, propyl, and isopropyl.

In certain embodiments of compounds of Formulae (II)-(VII), each of R² and R¹² is hydrogen; R³ is methyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formulae (II)-(VII), each of R² and R¹² is hydrogen; R³ is methyl; and R¹ is selected from methyl, ethyl, propyl, isopropyl, phenyl, benzyl, phenethyl, cyclopentyl, cyclohexyl, and 3-pyridyl. In certain embodiments of compounds of Formulae (II)-(VII), each of R² and R¹² is hydrogen; R³ is methyl; and R¹ is selected from methyl, ethyl, propyl, and isopropyl.

In certain embodiments of compounds of Formulae (II)-(VII), each of R² and R¹² is hydrogen; R³ is ethyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formulae (II)-(VII), each of R² and R¹² is hydrogen; R³ is ethyl; and R¹ is selected from methyl, ethyl, propyl, isopropyl, phenyl, benzyl, phenethyl, cyclopentyl, cyclohexyl, and 3-pyridyl. In certain embodiments of compounds of Formulae (II)-(VII), each of R² and R¹² is hydrogen; R³ is ethyl; and R¹ is selected from methyl, ethyl, propyl, and isopropyl.

In certain embodiments of compounds of Formulae (II)-(VII), each of R² and R¹² is hydrogen; R³ is propyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formulae (II)-(VII), each of R² and R¹² is hydrogen; R³ is propyl; and R¹ is selected from methyl, ethyl, propyl, isopropyl, phenyl, benzyl, phenethyl, cyclopentyl, cyclohexyl, and 3-pyridyl. In certain embodiments of compounds of Formulae (II)-(VII), each of R² and R¹² is hydrogen; R³ is propyl; and R¹ is selected from methyl, ethyl, propyl, and isopropyl.

In certain embodiments of compounds of Formulae (II)-(VII), each of R² and R¹² is hydrogen; R³ is isopropyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl. In certain embodiments of compounds of Formulae (II)-(VII), each of R² and R¹² is hydrogen; R³ is isopropyl; and R¹ is selected from methyl, ethyl, propyl, isopropyl, phenyl, benzyl, phenethyl, cyclopentyl, cyclohexyl, and 3-pyridyl. In certain embodiments of compounds of Formulae (II)-(VII), each of R² and R¹² is hydrogen; R³ is isobutyl; and R¹ is selected from methyl, ethyl, propyl, and isopropyl.

In certain embodiments, a compound of Formula (I) is selected from:
7-[((1R)-1-acetyloxyethyl)oxycarbonyl](1S,6S,8S)-7-azabicyclo[4.3.0]nonane-8-carboxylic acid;
7-[((1S)-1-acetyloxyethyl)oxycarbonyl](1S,6S,8S)-7-azabicyclo[4.3.0]nonane-8-carboxylic acid;
7-[((1R)-1-propanoyloxyethyl)oxycarbonyl](1S,6S,8S)-7-azabicyclo[4.3.0]nonane-8-carboxylic acid;
7-[((1S)-1-propanoyloxyethyl)oxycarbonyl](1S,6S,8S)-7-azabicyclo[4.3.0]nonane-8-carboxylic acid;
7-{[(1R)-1-(2-methylpropanoyloxy)ethyl]oxycarbonyl}(1S,6S,8S)-7-azabicyclo[4.3.0]nonane-8-carboxylic acid;
7-{[(1S)-1-(2-methylpropanoyloxy)ethyl]oxycarbonyl}(1S,6S,8S)-7-azabicyclo[4.3.0]nonane-8-carboxylic acid;
7-[((1R)-1-acetyloxy-2-methylpropyl)oxycarbonyl](1S,6S,8S)-7-azabicyclo[4.3.0]nonane-8-carboxylic acid;
7-[((1S)-1-acetyloxy-2-methylpropyl)oxycarbonyl](1S,6S,8S)-7-azabicyclo[4.3.0]nonane-8-carboxylic acid;
7-[((1R)-2-methyl-1-propanoyloxypropyl)oxycarbonyl](1S,6S,8S)-7-azabicyclo[4.3.0]nonane-8-carboxylic acid;
7-[((1S)-2-methyl-1-propanoyloxypropyl)oxycarbonyl](1S,6S,8S)-7-azabicyclo[4.3.0]nonane-8-carboxylic acid;
7-{[(1R)-2-methyl-1-(2-methylpropanoyloxy)propyl]oxycarbonyl}(1S,6S,8S)-7-azabicyclo[4.3.0]nonane-8-carboxylic acid;
7-{[(1S)-2-methyl-1-(2-methylpropanoyloxy)propyl]oxycarbonyl}(1S,6S,8S)-7-azabicyclo[4.3.0]nonane-8-carboxylic acid; and

a pharmaceutically acceptable salt of any of the foregoing.

In certain embodiments of a compound of Formula (I), the compound is selected from:
2-{[((1R)-1-acetyloxyethoxy)carbonylamino]methyl}-5-chlorobenzoic acid;
2-{[((1S)-1-acetyloxyethoxy)carbonylamino]methyl}-5-chlorobenzoic acid;
2-{[((1R)-1-propanoyloxyethoxy)carbonylamino]methyl}-5-chlorobenzoic acid;
2-{[((1S)-1-propanoyloxyethoxy)carbonylamino]methyl}-5-chlorobenzoic acid;
2-({[(1R)-1-(2-methylpropanoyloxy)ethoxy]carbonylamino}methyl)-5-chlorobenzoic acid;
2-({[(1S)-1-(2-methylpropanoyloxy)ethoxy]carbonylamino}methyl)-5-chlorobenzoic acid;
2-{[((1R)-1-acetyloxy-2-methylpropoxy)carbonylamino]methyl}-5-chlorobenzoic acid;
2-{[((1S)-1-acetyloxy-2-methylpropoxy)carbonylamino]methyl}-5-chlorobenzoic acid;
2-{[((1R)-1-(2-methyl-1-propanoyloxypropoxy)carbonylamino]methyl}-5-chlorobenzoic acid;
2-{[((1S)-1-(2-methyl-1-propanoyloxypropoxy)carbonylamino]methyl}-5-chlorobenzoic acid;
2-({[(1R)-2-methyl-1-(2-methylpropanoyloxy)propoxy]carbonylamino}methyl)-5-chlorobenzoic acid;
2-({[(1S)-2-methyl-1-(2-methylpropanoyloxy)propoxy]carbonylamino}methyl)-5-chlorobenzoic acid; and

a pharmaceutically acceptable salt of any of the foregoing.

### Synthesis of Compounds

Those skilled in the art will appreciate that compounds of Formula (I)-(VII) may be synthesized by attaching promoieties to alpha-2-delta ligands. Methods for synthesizing alpha-2-delta ligands are known in the art. Other methods for synthesizing alpha-2-delta ligands will be apparent to those skilled in the art in view of published references. Acyloxyalkyl carbamate promoieties provided by the present disclosure are known in the art and may be prepared and attached to alpha-2-delta ligands by established procedures (Gogate et al., International Journal of Pharmaceutics 1987, 40, 235-248; Alexander et al., J. Med. Chem. 1988, 31, 318-322; Sun et al., Bioorganic & Medicinal Chemistry Letters 2001, 11, 1875-1879; Alexander et al., J. Med. Chem. 1991, 34, 78-81; U.S. Pat. No. 4,760,057; U.S. Pat. No. 4,916,230; and U.S. Pat. No. 5,684,018). In certain embodiments, acyloxyalkyl carbamate promoieties provided by the present disclosure may be attached to alpha-2-delta ligands using the procedures disclosed in U.S. Pat. No. 6,818,787; U.S. Pat. No. 6,927,036; U.S. Pat. No. 6,972,341; U.S. Pat. No. 7,186,855; U.S. Pat. No. 7,026,351; U.S. Pat. No. 7,109,239; U.S. Pat. No. 7,227,028; U.S. Pat. No. 7,232,924; WO 2005/010011; and U.S. patent application Ser. Nos. 12/537,798 filed Aug. 7, 2009, and 12/537,764 filed Aug. 7, 2009. In certain embodiments, compounds of Formula (I) can be prepared by reacting an alpha-2-delta ligand of Formula (VIII) with an N-hydroxysuccinimidyl-acyloxyalkylcarbonate of Formula (IX):

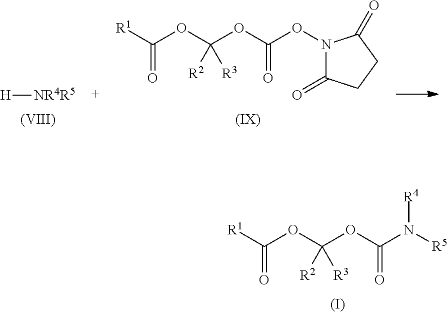

A feature common to such methods for synthesizing acyloxyalkyl derivatives is that the prodrugs are generated as racemates or diastereomeric mixtures except when both the R² and R³ substituents are identical (and typically both hydrogen). The presence of an additional chiral center in the promoiety may result in differences in the physical properties, pharmacokinetics, and/or efficacy of the prodrug among the diastereomers. As disclosed in US 2009/0192325 it is possible to prepare the promoiety synthon (IX) in substantially enantiopure form via enzymatic resolution of an acyloxyalkylthiocarbonate precursor of compound (IX) and thereby facilitate the preparation of compounds of Formula (I) as a substantially single diastereomer.

### Therapeutic Uses

Compounds of Formulae (I)-(VII), their pharmaceutically acceptable salts, and the corresponding parent alpha-2-delta ligands can be used to treat epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, and gastric damage in a human. The treatment of such diseases and disorders comprises administering to a patient in need of such treatment a therapeutically effective amount of a compound of Formulae (I)-(VII), or a pharmaceutically acceptable salt thereof.

Compounds of Formula (I)-(VII) and their pharmaceutically acceptable salts can be used to treat neurodegenerative disorders such as stroke, head trauma, asphyxia, spinal cord trauma, or injury from general anoxia, hypoxia, hypoglycemia, hypotension as well as similar injuries seen during procedures from embole, hyperfusion, and hypoxia. Such treatment is also useful in preventing neuronal damage that occurs during cardiac bypass surgery, in incidents of intracranial hemorrhage, in perinatal asphyxia, in cardiac arrest, and status epilepticus.

Compounds of Formula (I)-(VII) and their pharmaceutically acceptable salts can be used to treat delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorder, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; Down's syndrome; demyelinating diseases such as multiple sclerosis (MS) and amylolateral sclerosis (ALS), peripheral neuropathy, for example diabetic and chemotherapy-induced-neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid hemorrhage or cerebral edema in a human. The treatment of such diseases comprises administering to a patient in need of such treatment a therapeutically effective amount of a compound of Formulae (I)-(VII), or a pharmaceutically acceptable salt thereof.

Compounds of Formulae (I)-(VII) and their pharmaceutically acceptable salts can be used to treat pain, including pain resulting from soft tissue and peripheral damage, such as acute trauma, pain associated with osteoarthritis and rheumatoid arthritis, musculoskeletal pain, such as pain experienced after trauma; spinal pain, dental pain, myofascial pain syndromes, episiotomy pain, and pain resulting from burns; deep and visceral pain, such as heart pain, muscle pain, eye pain, orofacial pain, for example, odontalgia, abdominal pain, gynecological pain, for example, dysmenorrhea, labor pain and pain associated with endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, trigeminal neuralgia, neuropathic lower back pain, HIV-related neuropathic pain, cancer related neuropathic pain, diabetic neuropathic pain, and arachnoiditis; neuropathic and non-neuropathic pain associated with carcinoma, often referred to as cancer pain; central nervous system pain, such as pain due to spinal cord or brain stem damage; lower back pain; sciatica; phantom limb pain, headache, including migraine and other vascular headaches, acute or chronic tension headache, cluster headache, temperomandibular pain and maxillary sinus pain; pain resulting from ankylosing spondylitis and gout; pain caused by increased bladder contractions; post operative pain; scar pain; and chronic non-neuropathic pain such as pain associated with fibromyalgia, HIV, rheumatoid and osteoarthritis, anthralgia and myalgia, sprains, strains and trauma such as broken bones; and post surgical pain. Still other pain is caused by injury or infection of peripheral sensory nerves. It includes, but is not limited to pain from peripheral nerve trauma, herpes virus infection, diabetes mellitus, fibromyalgia, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Psychogenic pain is that which occurs without an organic origin such as low back pain, atypical facial pain, and chronic headache.

The compounds of Formulae (I)-(VII) and their pharmaceutically acceptable salts are also useful in the treatment of mood disorders, such as depression, or more particularly, depressive disorders, for example, single episodic or recurrent major depressive disorders, dysthymic disorders, depressive neurosis and neurotic depression, melancholic depression, including anorexia, weight loss, insomnia, early morning waking and psychomotor retardation, atypical depression (or reactive depression), including increased appetite, hypersomnia, psychomotor agitation or irritability, seasonal affective disorder and pediatric depression; or bipolar disorders or manic depression, for example, bipolar I disorder, bipolar II disorder and cyclothymic disorder; conduct disorder and disruptive behavior disorder; anxiety disorders, such as panic disorder with or without agoraphobia, agoraphobia without history of panic disorder, specific phobias, for example, specific animal phobias, social anxiety, social phobia, obsessive-compulsive disorder, stress disorders, including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder, mood disorders associated with schizophrenia; behavioral disturbances associated with mental retardation, autistic disorder, and conduct disorder in a human. The treatment of such diseases and disorders comprises administering to a patient in need of such treatment a therapeutically effective amount of a compound of Formulae (I)-(VII), or a pharmaceutically acceptable salt thereof.

The compounds of Formulae (I)-(VII) and their pharmaceutically acceptable salts are also useful in the treatment of sleep disorders. Sleep disorders are disturbances that affect the ability to fall and/or stay asleep, that involves sleeping too much, or that result in abnormal behavior associated with sleep. The disorders include, for example, insomnia, drug-associated sleeplessness, hypersomnia, restless legs syndrome, narcolepsy, sleep apnea syndromes, and parasomnias. Treatment of such sleep disorders comprise administering to a patient in need of such treatment a therapeutically effective amount of a compound of Formulae (I)-(VII), or a pharmaceutically acceptable salt thereof.

The suitability of the compounds and/or pharmaceutical compositions disclosed herein in treating or preventing epilepsy, depression, anxiety, psychosis, faintness attacks, hypokinesia, cranial disorders, neurodegenerative disorders, panic, pain (especially neuropathic pain and muscular and skeletal pain), inflammatory disease (i.e., arthritis), insomnia, gastrointestinal disorders and ethanol withdrawal syndrome may be determined by methods described in the art (see, e.g., U.S. Pat. No. 4,024,175; U.S. Pat. No. 4,087,544; U.S. Pat. No. 5,084,169; U.S. Pat. No. 5,563,175; U.S. Pat. No. 6,001,876; U.S. Pat. No. 6,020,370; U.S. Pat. No. 6,028,214; U.S. Pat. No. 6,103,932; U.S. Pat. No. 6,117,906; WO 92/09560; WO 93/23383; WO 97/29101; WO 97/33858; WO 97/33859; WO 98/17627; WO 99/08671; WO 99/21824; WO 99/31057; WO 99/37296; WO 99/31075; WO 99/61424; WO 00/23067; WO 00/31020; WO 00/50027; WO 02/00209; WO 01/28978; and WO 02/085839).

Accordingly, it is well within the capability of those of skill in the art to assay and use the compounds of this disclosure and/or pharmaceutical compositions thereof to treat or prevent the above diseases or disorders.

### Therapeutic Administration

The compounds and/or pharmaceutical compositions disclosed herein may be used in human medicine. Compounds and/or pharmaceutical compositions of compounds of Formulae (I)-(VII) are useful for the treatment or prevention of epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, and gastric damage in a human.

When used to treat or prevent the above disease or disorders compounds and/or pharmaceutical compositions of compounds of Formulae (I)-(VII) may be administered or applied singly, in combination with another compound of Formulae (I)-(VII) and/or in combination with other agents. These compounds and/or pharmaceutical compositions may also be administered or applied singly, in combination with other pharmaceutically active agents, including other compounds and/or pharmaceutical compositions disclosed herein.

The current disclosure provides methods of treatment and prophylaxis by administration to a patient of a therapeutically effective amount of a pharmaceutical composition or compound of Formulae (I)-(VII). The patient may be an animal, in some embodiments a mammal, and in some embodiments a human.

The present compounds and/or pharmaceutical compositions of this disclosure may be administered orally. These compounds and/or pharmaceutical compositions may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known (e.g., encapsulation in liposomes, microparticles, microcapsules, capsules, etc.) that can be used to administer a compound and/or composition of Formulae (I)-(VII). Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

In certain embodiments, the compounds and/or pharmaceutical compositions of compounds of Formulae (I)-(VII) can be delivered via sustained release systems, and in certain embodiments, oral sustained release systems.

### Modes of Administration

A compound of Formulae (I)-(VII) or a pharmaceutical composition thereof may be advantageously used in human medicine. As disclosed herein, a compound of Formulae (I)-(VII) or a pharmaceutical composition thereof are useful for the treatment of epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, and gastric damage.

When used to treat the above diseases, a compound of Formulae (I)-(VII) or a pharmaceutical composition thereof may be administered or applied singly, or in combination with other agents. A compound of Formulae (I)-(VII) or a pharmaceutical composition thereof may also be administered or applied singly or in combination with other pharmaceutically active agents, including other alpha-2-delta ligands.

Methods of treatment include administering to a patient in need of such treatment a therapeutically effective amount of a compound of Formulae (I)-(VII) or a pharmaceutical composition thereof. The patient may be an animal, such as a mammal, for example, a human.

In certain embodiments, a compound of Formulae (I)-(VII) or a pharmaceutical composition thereof can be administered orally. A compound of Formulae (I)-(VII) or a pharmaceutical composition thereof may be administered by any other convenient route, for example, by infusion or bolus injection, or by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound of Formulae (I)-(VII) or a pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes or skin.

In certain embodiments, a compound of Formulae (I)-(VII) or a pharmaceutical composition thereof may be delivered via sustained release systems, such as an oral sustained release system.

A compound of Formulae (I)-(VII) or a pharmaceutical composition thereof provides the corresponding alpha-2-delta ligand upon in vivo administration to a patient. A compound of Formulae (I)-(VII) or a metabolite thereof including the corresponding alpha-2-delta ligand may be absorbed into the systemic circulation from the gastrointestinal tract either by passive diffusion, active transport or by both passive and active processes.

The promoiety of a compound of Formulae (I)-(VII) may be cleaved either chemically and/or enzymatically. One or more enzymes present in the stomach, intestinal lumen, intestinal tissue, blood, liver, brain or any other tissue of a mammal may cleave the promoiety of a compound of Formulae (I)-(VII). The promoiety of a compound of Formulae (I)-(VII) may be cleaved prior to absorption by the gastrointestinal tract (e.g., within the stomach or intestinal lumen) and/or after absorption by the gastrointestinal tract (e.g., in intestinal tissue, blood, liver or other suitable tissue of a mammal). When the promoiety of a compound of Formulae (I)-(VII) is cleaved prior to absorption by the gastrointestinal tract, the corresponding alpha-2-delta ligand may be absorbed into the systemic circulation conventionally (e.g., mediated, in part, via the large neutral amino acid transporter located in the small intestine). When the promoiety of a compound of Formulae (I)-(VII) is cleaved after absorption by the gastrointestinal tract, the corresponding alpha-2-delta ligand may be absorbed into the systemic circulation either by passive diffusion, active transport, or by both passive and active processes.

When the promoiety of a compound of Formulae (I)-(VII) is cleaved after absorption by the gastrointestinal tract, the compound may be absorbed into the systemic circulation from the large intestine. When the compound of Formulae (I)-(VII) is absorbed by the large intestine, a compound of Formulae (I)-(VII) or a pharmaceutical composition thereof can be advantageously administered as a sustained release system. In certain embodiments, a compound of Formulae (I)-(VII) or pharmaceutical composition thereof can be delivered by oral sustained release administration. When administered using a sustained release formulation, a compound of Formulae (I)-(VII) or a pharmaceutical composition thereof can be administered twice per day or once per day.

In certain embodiments, oral administration of an oral sustained release dosage form comprising a compound of Formulae (I)-(VII) can provide a therapeutically effective concentration of the corresponding alpha-2-delta ligand in the blood plasma of a patient for a time period of at least about 4 hours after administration of the dosage form, in certain embodiments, for a time period of at least about 8 hours, and in certain embodiments, for a time period of at least about 12 hours, and in certain embodiments, for a time period of at least about 24 hours.

### Therapeutic Doses

A compound of Formulae (I)-(VII) or a pharmaceutical composition thereof, can generally be used in an amount effective to achieve the intended purpose such as for use to treat diseases or disorders such as epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage. A compound of Formulae (I)-(VII), or a pharmaceutical composition thereof can be administered in a therapeutically effective amount.

The amount of a compound of Formulae (I)-(VII) or a pharmaceutical composition thereof that will be effective in the treatment of a particular disease or disorder will depend on the nature of the disorder or condition, and can be determined by standard clinical techniques known in the art as previously described. In addition, in vitro or in vivo assays may optionally be employed to help identify optimal dosage ranges. The amount of a compound of Formulae (I)-(VII) or a pharmaceutical composition thereof administered will depend on, among other factors, the subject being treated, the weight of the subject, the severity of the affliction, the manner of administration and/or the judgment of the prescribing physician.

A dose may be delivered in a pharmaceutical composition by a single administration or by multiple applications of one or more dosage forms. In certain embodiments, a compound of Formulae (I)-(VII) or a pharmaceutical composition thereof can be delivered by oral sustained release administration. A sustained release formulation comprising a compound of Formulae (I)-(VII) or a pharmaceutical composition thereof can be administered twice per day or once per day. Dosing may be repeated intermittently, may be provided alone or in combination with other drugs, and may continue as long as required for effective treatment of the disease or disorder.

In certain embodiments, a dose or multiple doses of a compound of Formulae (I)-(VII) or a pharmaceutical composition thereof can provide between about 10 mg/day and about 2,000 mg/day of the corresponding alpha-2-delta ligand, in certain embodiments between about 50 mg/day and about 1,000 mg/day of the corresponding alpha-2-delta ligand, and in certain embodiments, between about 100 mg/day and about 600 mg/day of the corresponding alpha-2-delta ligand. Appropriate dosage ranges for treating a particular disease may be readily determined by methods known to those skilled in the art.

A compound of Formulae (I)-(VII) or a pharmaceutical composition thereof can be assayed in vitro and in vivo, for the desired therapeutic or prophylactic activity, prior to use in humans. A therapeutically effective dose of a compound of Formulae (I)-(VII) or a pharmaceutical composition thereof can provide therapeutic benefit without causing substantial toxicity and adverse side effects. Toxicity and adverse side effects of a compound of Formulae (I)-(VII), a pharmaceutical composition thereof, and metabolites thereof may be determined using standard pharmaceutical procedures and may be readily ascertained by the skilled artisan. The dose ratio between toxic and adverse side effects and therapeutic effect is the therapeutic index. A dose of a compound of Formulae (I)-(VII) or a pharmaceutical composition thereof can provide a circulating concentration of the corresponding alpha-2-delta ligand that is within a therapeutically effective concentration with little or no toxicity or adverse side effects.

### Combination Therapy

In certain embodiments, a compound of Formulae (I)-(VII) or a pharmaceutical composition thereof can be used in combination therapy with at least one other therapeutic agent. A compound of Formulae (I)-(VII) or a pharmaceutical composition thereof and the at least one other therapeutic agent can act additively or synergistically. In certain embodiments, a pharmaceutical composition comprising a compound of Formulae (I)-(VII) can be administered concurrently with the administration of another therapeutic agent, which can be part of the same pharmaceutical composition as a compound of Formulae (I)-(VII) or the other therapeutic agent can be in a different pharmaceutical composition. In certain embodiments, a pharmaceutical composition comprising a compound of Formulae (I)-(VII) can be administered prior to or subsequent to administration of another therapeutic agent.

In certain embodiments, a compound of Formulae (I)-(VII) may be used singly or in combination with another compound of Formulae (I)-(VII).

The additional therapeutic agent may be effective for treating epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage. In certain embodiments in which a compound of Formulae (I)-(VII) is administered together with an additional therapeutic agent for treating epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage, each of the active agents may be used at lower doses than when used singly.

The weight ratio of a compound of Formulae (I)-(VII) to a second therapeutic agent may be varied and may depend upon the effective dose of each agent. A therapeutically effective dose of each compound can be used. Thus, for example, when a compound of Formulae (I)-(VII) is combined with another therapeutic agent, the weight ratio of the compound provided by the present disclosure to the second therapeutic agent can be from about 1000:1 to about 1:1000, from about 200:1 to about 1:200, from about 20:1 to about 1:20, and in certain embodiments, from about 50:1 to about 1:5.

Combinations of a compound of Formulae (I)-(VII) and a second therapeutic agent may also be within the aforementioned range, but in each case, an effective dose of each active compound can be used. In such combinations a compound of Formulae (I)-(VII) and second therapeutic agent may be administered separately or in conjunction. In addition, the administration of one element may be prior to, concurrent with, or subsequent to the administration of another therapeutic agent(s). Accordingly, a compound of Formulae (I)-(VII) may be used alone or in combination with other therapeutic agents that are known to be beneficial in treating epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage, or other therapeutic agents that affect receptors or enzymes that either increase the efficacy, safety, convenience, or reduce unwanted side effects or toxicity of a compound of Formulae (I)-(VII) and/or metabolites thereof. A compound of Formulae (I)-(VII) and the other therapeutic agent may be co-administered, either in concomitant therapy or in a fixed combination. The additional therapeutic agent may be administered by the same or different route than the route used to administer a compound of Formulae (I)-(VII) or pharmaceutical composition thereof.

In certain embodiments, a compound of Formulae (I)-(VII) or a pharmaceutical composition thereof may be administered to a patient for the treatment of epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage in combination with a therapy or therapeutic agent known or believed to be effective in the treatment of epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage, respectively, or in certain embodiments, a disease, disorder, or condition associated with epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, or gastric damage, respectively.

In certain embodiments, a compound of Formulae (I)-(VII) or a pharmaceutical composition thereof may be administered to a patient for treating migraine in combination with a therapy or another therapeutic agent known or believed to be effective in treating migraine. Drugs useful for treating migraine can prevent a migraine from occurring, abort a migraine that is beginning, or relieve pain during the migraine episode.

Prophylactic migraine treatments reduce the frequency of migraines and include non-steroidal anti-inflammatory agents (NSAIDs), adrenergic beta-blockers, calcium channel blockers, tricyclic antidepressants, selective serotonin reuptake inhibitors, anticonvulsants, NMDA receptor antagonists, angiotensin converting enzyme (ACE) inhibitors, angiotensin-receptor blockers (ARBs), leukotriene-antagonists, dopamine agonists, selective 5HT-1D agonists, selective 5HT-1F agonists, AMPA/KA antagonists, CGRP (calcitonin gene related peptide) antagonists, NOS (nitric oxide synthase) inhibitors, blockers of spreading cortical depression, and other therapy. Examples of NSAIDs useful for preventing migraine include aspirin, ibuprofen, fenoprofen, flurbiprofen, ketoprofen, mefenamic acid, and naproxen. Examples of adrenergic beta-blockers useful for preventing migraine include acebutolol, atenolol, iminol, metoprolol, nadolol, pindolol, propranolol, and timolol. Examples of calcium channel blockers useful for preventing migraine include amlodipine, diltiazem, dotarizine, felodipine, flunarizine, nicardipine, nifedipine, nimodipine, nisoldipine, and verapamil. Examples of tricyclic antidepressants useful for preventing migraine include amitriptyline, desipramine, doxepin, imipramine, nortriptyline, and protriptyline. Examples of selective serotonin reuptake inhibitors (SSRIs) useful for preventing migraine include fluoxetine, methysergide, nefazodone, paroxetine, sertraline, and venlafaxine. Examples of other antidepressants useful for preventing migraine include bupropion, nefazodone, norepinephrine, and trazodone.

Examples of anticonvulsants (antiepileptics) useful for preventing migraine include divalproex sodium, felbamate, gabapentin, lamotrigine, levetiracetam, oxcarbazepine, tiagabine, topiramate, valproate, and zonisamide. Examples of NMDA receptor antagonists useful for preventing migraine include dextromethorphan, magnesium, and ketamine. Examples of angiotensin converting enzyme (ACE) inhibitors useful for preventing migraine include lisinopril. Examples of angiotensin-receptor blockers (ARBs) useful for preventing migraine include candesartan. Examples of leukotriene-antagonists useful for preventing migraine include zileuton, zafirlukast, montelukast, and pranlukast. Examples of dopamine agonists useful for preventing migraine include α-dihydroergokryptine. Examples of other therapy useful for preventing migraine include botulinum toxin, magnesium, hormone therapy, riboflavin, methylergonovine, cyproheptadine, and phenelzine, and complementary therapies such as counseling/psychotherapy, relaxation training, progressive muscle relaxation, guided imagery, diaphragmatic breathing, biofeedback, acupuncture, and physical and massage therapy.

Acute migraine treatments intended to eliminate or reduce the severity of the headache and any associated symptoms after a migraine has begun include serotonin receptor agonists, such as triptans (5-hydroxytryptophan (5-HT) agonists) such as almotriptan, eletriptan, frovatriptan, naratriptan, rizatriptan, sumatriptan, and zolmitriptan; ergotamine-based compounds such as dihydroergotamine and ergotamine; antiemetics such as metoclopramide and proclorperazine; and compounds that provide analgesic effects.

Other examples of drugs used to treat migraine once started include acetaminophen, aspirin, caffeine, cyproheptadine, methysergide, valproic acid, NSAIDs such as diclofenac, flurbiprofen, ketoprofen, ketorolac, ibuprofen, indomethacin, meclofenamate, and naproxen sodium, opioids such as codeine, meperidine, and oxycodone, and glucocorticoids including dexamethasone, prednisone and methylprednisolone.

A compound of Formulae (I)-(VII) may also be administered in conjunction with drugs that are useful for treating symptoms associated with migraine such as nausea and vomiting, and depression. Examples of useful therapeutic agents for treating or preventing vomiting include, but are not limited to, 5-HT₃ receptor antagonists such as ondansetron, dolasetron, granisetron, and tropisetron; dopamine receptor antagonists such as proclorperazine, thiethylperazine, chlorpromazine, metoclopramide, and domperidone; glucocorticoids such as dexamethasone; and benzodiazepines such as lorazepam and alprazolam. Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as fluoxetine, fluvoxamine, paroxetine, and setraline; monoamine oxidase inhibitors such as isocarboxazid, pargyline, phenizine, and tranylcypromine; and psychostimulants such as dextroamphetamine and methylphenidate.

In certain embodiments, a compound of Formulae (I)-(VII) or a pharmaceutical composition thereof may be administered to a patient for treating neuropathic pain in combination with a therapy or another therapeutic agent known or believed to be effective in treating neuropathic pain. Examples of drugs useful for treating pain include opioid analgesics such as morphine, codeine, fentanyl, meperidine, methadone, propoxyphene, levorphanol, hydromorphone, oxycodone, oxymorphone, tramadol and pentazocine; non-opioid analgesics such as aspirin, ibuprofen, ketoprofen, naproxen, and acetaminophen; non-steroidal anti-inflammatory drugs such as aspirin, choline magnesium trisalicylate, diflunisal, salsalate, celecoxib, rofecoxib, valdecoxib, diclofenac, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, sulindac, and tometin; antiepileptics such as gabapentin, pregabalin, carbamazepine, phenyloin, lamotrigine, and topiramate; antidepressants such as duloxetine, amitriptyline, venlafaxine, nortriptyline, imipramine, and desipramine; local anesthetics such as lidocaine, and mexiletine; NMDA receptor antagonists such as dextromethorphan, memantine, and ketamine; N-type calcium-channel blockers such as ziconotide; vanilloid receptor-1 modulators such as capsaicin; cannabinoid receptor modulators such as sativex; neurokinin receptor antagonists such as lanepitant; other analgesics such as neurotropin; and other drugs such as desipramine, clonazepam, divalproex, oxcarbazepine, divalproex, butorphanol, valdecoxib, vicoprofen, pentazocine, propoxyphene, fenoprofen, piroxicam, indomethacin, hydroxyzine, buprenorphine, benzocaine, clonidine, flurbiprofen, meperidine, lacosamide, desvenlafaxine, and bicifadine.

In certain embodiments, a drug useful for treating neuropathic pain is chosen from propoxyphene, meperidine, hydromorphone, hydrocodone, morphine, codeine, 2-piperidinol-1-alkanol, eliprodil, ifenprodil, rofecoxib, celecoxib, salicylic acid, diclofenac, piroxicam indomethacin, ibuprofen, naproxen, gabapentin, carbamazepine, pregabalin, topiramate, valproic acid, sumatriptan, eletriptan, rizatriptan, zolmitriptan, naratriptan, flexeril, carisoprodol, robaxisal, norgesic, dantrium, diazepam, chlordiazepoxide, alprazolam, lorazepam, acetaminophen, nitrous oxide, halothane, lidocaine, etidocaine, ropivacaine, chloroprocaine, sarapin, bupivacaine, capsaicin, desipramine, amitriptyline, doxepin, perphenazine, protriptyline, tranylcypromine, baclofen, clonidine, mexelitine, diphenhydramine, hydroxyzine, caffeine, prednisone, methyl-prednisone, decadron, sertraline, paroxetine, fluoxetine, tramadol, levodopa, dextromethorphan, substance P antagonists, and botulinum toxin. In certain embodiments, a drug useful for treating neuropathic pain can be chosen from a nicotine receptor partial agonist. Non-pharmacological therapies for treating neuropathic pain include transcutaneous electrical nerve stimulation, percutaneous electrical nerve stimulation, and acupuncture.

In certain embodiments, a compound of Formulae (I)-(VII) or a pharmaceutical composition thereof may be administered to a patient for treating post-herpetic neuralgia in combination with a therapy or another therapeutic agent known or believed to be effective in treating post-herpetic neuralgia. Examples of drugs useful for treating post-herpetic neuralgia include antiviral agents such as amantadine, acyclovir, cidofovir, desciclovir, deoxyacyclovir, famciclovir, foscamet, ganciclovir, penciclovir, azidouridine, ansamycin, amantadine, bromovinyldeoxusidine, chlorovinyldeoxusidine, cytarabine, didanosine, deoxynojirimycin, dideoxycytidine, dideoxyinosine, dideoxynucleoside, edoxudine, enviroxime, fiacitabine, foscamet, fluorothymidine, floxuridine, hypericin, interferon, interleukin, isethionate, nevirapine, pentamidine, ribavirin, rimantadine, stavirdine, sargramostin, suramin, trichosanthin, tribromothymidine, trichlorothymidine, vidarabine, zidoviridine, zalcitabine 3-azido-3-deoxythymidine, 2′,3′-dideoxyadenosine (ddA), 2′,3′-dideoxyguanosine (ddG), 2′,3′-dideoxycytidine (ddC), 2′,3′-dideoxythymidine (ddT), 2′,3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′,3′-dideoxy-2′,3′-didehydro-2′-fluorothymidine (Fd4T), 2′,3′-dideoxy-2′-beta-fluoroadenosine (F-ddA), 2′,3′-dideoxy-2′-beta-fluoro-inosine (F-ddI), and 2′,3′-dideoxy-2′-beta-fluorocytosine (F-ddC), trisodium phosphomonoformate, trifluorothymidine, 3′ azido-3′ thymidine (AZT), dideoxyinosine (ddI), and idoxuridine.

A compound of Formulae (I)-(VII) or pharmaceutical composition thereof may be administered singly or in combination with another therapeutic agent used to treat or manage chronic pain in a patient. Chronic pain is pain that persists for three months or more and includes low back pain, muscle pain, cancer pain, arthritis pain, osteoarthritis pain, osteoporosis pain, fibromyalgia, chronic neuropathic pain, chronic postoperative pain, pain associated with inflammatory bowel disease, pain associated with irritable bowel syndrome, and pain associated with rheumatoid arthritis.

Examples of agents used to treat or manage chronic pain include, for example, acetaminophen, amitriptyline, amitriptyline, aspirin, butorphanol, celecoxib, choline salicylate, diclofenac, diflunisal, duloxetine, etodolac, fentanyl, gabapentin, hydromorphone, hydroxyzine, ibuprofen, imipramine, indomethacin, ketoprofen, lidocaine, meperidine, methadone, morphine, nalbuphine, naproxen, oxycodone, oxymorphone, pentazocine, pramoxine, pregabalin, propoxyphene, rofecoxib, tapentadol, tolmetin, tramadol, trolamine salicylate, and valdecoxib.

In certain embodiments, a compound of Formulae (I)-(VII) may be administered together with an opioid agonist to treat chronic pain in a patient. Examples of opioid agonists include a phenanthrene such as codeine, morphine, thebaine, oripavine; a semisynthetic derivative such as diacetylmorphine (heroin), dihydrocodeine, hydrocodone, hydromorphone, nicomorphine, oxycodone, and oxymorphone; an anilidopiperidine such as fentanyl, alphamethylfentanyl, alfentanil, sufentanil, remifentanil, carfentanyl, and ohmefentanyl; a phenylpiperidine such as pethidine (meperidine), ketobemidone, MPPP, allylprodine, prodine, and PEPAP; a diphenylpropylamine derivative such as propoxyphene, dextropropoxyphene, dextromoramide, bezitramide, piritramide, methadone, dipipanone, levomethadyl acetate (LAAM), loperamide, and diphenoxylate; a benzomorphan derivative such as dezocine, pentazocine, and phenazocine; an oripavine derivative such as buprenorphine, dihydroetorphine and etorphine; a morphinan derivative such as butorphanol, nalbuphine, levorphanol, and levomethorphan; and others such as lefetamine, meptazinol, tilidine, tramadol, and tapentadol. In certain embodiments, an opioid agonist is chosen from alfentanil, allylprodine, alphaprodine, anileridine, benzylmorphine, bezitramide, brifentanil, buprenorphine, butorphanol, carfentanil, clonitazene, codeine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, mirfentanil, morphine, morphine-6-glucuronide, myrophine, nalbuphine, narceine, nicomorphine, norlevorphanol, normethadone, nociceptin/orphanin FQ (N/OFQ), normorφphine, noφipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tapentadol, tramadol, trefentanil, and tilidine. Opioid agonists include compounds exhibiting an agonistic effect at an opioid receptor including a μ-, κ-, δ-, and/or nociceptin receptor. An opioid agonist may also exhibit agonist or antagonist activity at other receptors.

In certain embodiments, a compound of Formulae (I)-(VII) may be administered together with selective serotonin reuptake inhibitor (SSRI) to treat chronic pain in a patient. In certain embodiments, an SSRI is chosen from cericlamine, citalopram, cyanodothiepin D,L-fenfluramine, dapoxetine, demethylsertraline, desmethylcitalopram, escitalopram, femoxetine, fluoxetine, fluvoxamine, ifoxetine, litoxetine, nefazodone, norfluoxetine, paroxetine, sertraline, trazodone, and zimelidine. An SSRI functions by inhibiting the reuptake of serotonin by afferent neurons.

In certain embodiments, a compound of Formulae (I)-(VII) may be administered together with selective noradrenaline/norepinephrine reuptake inhibitor (SNRI) to treat chronic pain in a patient. In certain embodiments, an SNRI is chosen from atomoxetine, bicifadine, buproprion, desipramine, fezolamine, hydroxybuproprion, lofepramine, maprotiline, mianserin, sibutramine, mirtazepine, nomifensine, oxaprotiline, reboxetine, and viloxazine.

In certain embodiments, a compound of Formulae (I)-(VII) may be administered together with a compound that inhibits the reuptake of both serotonin and norepinephrine to treat chronic pain in a patient. In certain embodiments, a compound that inhibits the reuptake of both serotonin and norepinephrine is chosen from clomipramine, desmethylclomipramine, duloxetine, imipramine, milnacipran, O-desmethylvenlafaxine, and venlafaxine.

Other agents that may be co-administered with a compound of Formulae (I)-(VII) or pharmaceutical composition thereof for treating chronic pain include nonsteroidal antiinflammatory drugs (NSAIDs) such as aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, and zomepirac; barbiturate sedatives such as amobarbital, aprobarbital, butabarbital, butabital, mephobarbital, metharbital, methohexital, pentobarbital, phenobarbital, secobarbital, talbutal, theamylal, and thiopental; benzodiazepines such as chlordiazepoxide, clorazepate, diazepam, flurazepam, lorazepam, oxazepam, temazepam, and triazolam; H₁ antagonists such as diphenhydramine, pyrilamine, promethazine, chlorpheniramine, and chlorocyclizine; other sedatives such as glutethimide, meprobamate, methaqualone, and dichloralphenazone; skeletal muscle relaxants such as baclofen, carisoprodol, chlorzoxazone, cyclobenzaprine, methocarbamol, and orphrenadine; NMDA receptor antagonists such as dextromethorphan ((+)-3-hydroxy-N-methylmorphinan), dextrorphan ((+)-3-hydroxy-N-methylmorphinan), ketamine, memantine, pyrroloquinoline quinine, and cis-4-(phosphonomethyl)-2-piperidinecarboxylic acid; α-adrenergic active compounds such as doxazocin, tamsulosin, clonidine, and 4-amino-6,7-dimethoxy-2-(5-methanesulfonamido-1,2,3,4-tetra-hydroisoquinol-2-yl)-5-(2-pyridyl)quinazoline; tricyclic antidepressants such as amytriptiline, amoxapine, butriptyline, clomipramine, desipramine, dosulepin hydrochloride, doxepin, imipramine, iprindole, lofepramine, nortriptyline, opipramol, protriptyline, and trimipramine; anticonvulsants such as carbamazepine and valproate; tachykinin (NK) antagonists such as (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione, 5-[[(2R,3S)-2-[(1R)-1-[3,5-bis(trifluorom-ethyl)phenyl]ethoxy-3-(4-fluorophenyl)-4-morpholinyl]methyl]-1,2-dihydro-3-H-1,2,4-triazol-3-one, lanepitant, dapitant, and 3-[[2-methoxy-5-(trifluoromethoxy)phenyl]methylamino]-2-phenyl-piperidine (2S,3S); muscarinic antagonists such as oxybutin, tolterodine, propiverine, tropsium chloride, and darifenacin; COX-2 inhibitors such as celecoxib, rofecoxib and valdecoxib; non-selective COX inhibitors such as nitroflurbiprofen; coal-tar analgesics such as paracetamol; neuroleptics such as droperidol; vanilloid receptor agonists such as resiniferatoxin; β-adrenergic compounds such as propranolol; local anaesthetics such as mexiletine; corticosteroids such as dexamethasone; serotonin receptor agonists and antagonists; cholinergic (nicotinic) analgesics; and PDEV inhibitors, such as sildenafil, vardenafil, and taladafil.

A compound of Formulae (I)-(VII) and the other agent for treating pain such as an SSRI, SNRI, a compound that inhibits the reuptake of both serotonin and norepinephrine, or other agent for treating pain may be formulated in the same pharmaceutical composition or separate pharmaceutical compositions. In certain embodiments, a pharmaceutical composition comprising a compound of Formulae (I)-(VII) and the other agent for treating chronic pain is a sustained release formulation. In certain embodiments, the sustained release formulation may be adapted to be administered to a patient once per day or twice per day. In certain embodiments, a dosage form may comprise about 50 mg to about 1,200 mg of a compound of Formulae (I)-(VII), and about 10 mg to about 1,200 mg of the other agent for treating pain. In certain embodiments, the ratio of the amount of a compound of Formulae (I)-(VII) to the amount of the other agent for treating pain in a dosage form is from about 1:200 to about 200:1; from about 1:50 to about 50:1, from about 1:10 to about 10:1, and in certain embodiments from about 1:4 to about 4:1.

In certain embodiments, compounds of Formulae (I)-(VII) may be coadministered with baclofen or a baclofen prodrug such as (3R)-4-{[(1S)-2-methyl-1-(2-methylpropanoyloxy)propoxy]carbonylamino}-3-(4-chororphenyl)butanoic acid or other baclofen prodrugs disclosed in U.S. Pat. No. 7,109,239.

### Kits

A compound of Formulae (I)-(VII) or a pharmaceutical composition thereof may be included in a kit that may be used to administer the compound to a patient for treating a disease. A kit can include a pharmaceutical composition comprising a compound of Formulae (I)-(VII) suitable for administration to a patient and instructions for administering the pharmaceutical composition to a patient. A kit can include one or more containers for containing one or more pharmaceutical compositions and may include divided containers such as a divided bottle or a divided foil packet. A container can be any appropriate shape or form made of a pharmaceutically acceptable material. A particular container can depend on the dosage form and the number of dosage forms provided. Instructions provided with a kit can include directions for administration and may include a memory aid. Instructions supplied with a kit may be printed and/or supplied, for example, as an electronic-readable medium, a video cassette, an audiotape, a flash memory device, or may be published on an internet web site or distributed to a patient as an electronic mail. A memory aid may be a written memory aid, which contains information and/or instructions for the physician, pharmacist, and/or patient to facilitate compliance with a dosing regimen. A memory aid may also be mechanical or electronic. When a therapeutic regimen includes administration of a compound of Formulae (I)-(VII) and at least one other therapeutic agent, a kit can include the at least one other therapeutic agent in the same or separate container as the a compound of Formulae (I)-(VII).

In certain embodiments, a kit comprises a pharmaceutical composition comprising a compound of Formulae (I)-(VII) and instructions for administering the pharmaceutical composition to a patient in need thereof for treating a disease chosen from epilepsy, faintness attacks, fibromyalgia, restless legs syndrome, insomnia and other sleep disorders, hypokinesia, cranial disorders, hot flashes, essential tremor, overactive bladder, chemical dependencies and addictions, (e.g., dependencies on or addictions to alcohol, amphetamines, caffeine, cannabis, cocaine, heroin, hallucinogens, tobacco, inhalants and aerosol propellants, nicotine, opioids, sedatives, hypnotics, benzodiazepines and other anxiolytics), and withdrawal symptoms associated with such dependencies or addictions, addictive behaviors such as gambling, migraine, spasticity, arthritis, irritable bowel syndrome (IBS), chronic pain, acute pain, neuropathic pain, vascular headache, sinus headache, inflammatory disorders (e.g., rheumatoid arthritis, osteoarthritis, psoriasis) diuresis, premenstrual syndrome, premenstrual dysphoric disorder, tinnitus, and gastric damage.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A compound of Formula (II): 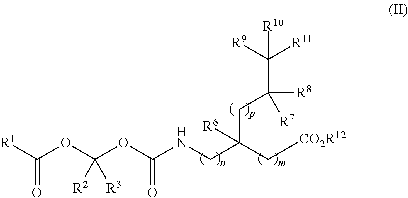
isomers thereof, and pharmaceutically acceptable salts of any of any of the foregoing, wherein:
each of m and n is 1;
p is selected from 0 and 1;
R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;
R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and a substituted C₃₋₈ heterocycloalkyl ring;
R⁶ is selected from hydrogen, methyl, fluoromethyl, difluoromethyl, and trifluoromethyl;
R⁷, R⁸, and R⁹ are independently selected from hydrogen and C₁₋₆ alkyl;
R¹⁰ and R¹¹ are independently selected from hydrogen and C₁₋₆ alkyl; or R¹⁰ and R¹¹ together with the carbon atom to which they are bonded form a C₃₋₆ cycloalkyl ring; and
R¹² is selected from hydrogen and C₁₋₆ alkyl.

2. The compound of claim 1, wherein the moiety having the formula: 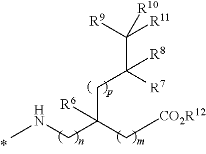
is derived from an alpha-2-delta ligand of the formula:

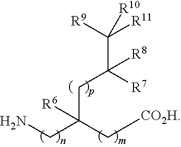

3. The compound of claim 2, wherein the alpha-2-delta ligand is selected from:
(3S,5R)-3-(aminomethyl)-5-methylheptanoic acid;
(3R,4R,5R)-3-(aminomethyl)-4,5-dimethylheptanoic acid;
(3S,5R)-3-(aminomethyl)-5-methyloctanoic acid;
(3R,4R,5R)-3-(aminomethyl)-4,5-dimethyloctanoic acid;
(3S,5R)-3-(aminomethyl)-5-methylnonanoic acid;
(3R,4R,5R)-3-(aminomethyl)-4,5-dimethylnonanoic acid;
(3S,5R)-3-(aminomethyl)-5-methyldecanoic acid; and
(3S,5R)-3-(aminomethyl)-5-methylundecanoic acid.

4. The compound of claim 1, wherein R² is hydrogen; R³ is selected from hydrogen, methyl, ethyl, propyl, and isopropyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl.

5. The compound of claim 1, wherein R² is hydrogen; R³ is selected from hydrogen, methyl, ethyl, propyl, and isopropyl; and R¹ is selected from methyl, ethyl, propyl, isopropyl, phenyl, benzyl, phenethyl, cyclopentyl, cyclohexyl, and 3-pyridyl.

6. The compound of claim 1, wherein R² is hydrogen; R³ is selected from hydrogen, methyl, ethyl, propyl, and isopropyl; and R¹ is selected from methyl, ethyl, propyl, and isopropyl.

7. The compound of claim 1, wherein the compound is selected from:
3-{[((1R)-1-acetyloxyethoxy)carbonylamino]methyl}(3S,5R)-5-methylheptanoic acid;
3-{[((1S)-1-acetyloxyethoxy)carbonylamino]methyl}(3S,5R)-5-methylheptanoic acid;
3-{[((1R)-1-propanoyloxyethoxy)carbonylamino]methyl}(3S,5R)-5-methylheptanoic acid;
3-{[((1S)-1-propanoyloxyethoxy)carbonylamino]methyl}(3S,5R)-5-methylheptanoic acid;
3-({[(1R)-1-(2-methylpropanoyloxy)ethoxy]carbonylamino}methyl)(3S,5R)-5-methylheptanoic acid;
3-({[(1S)-1-(2-methylpropanoyloxy)ethoxy]carbonylamino}methyl)(3S,5R)-5-methylheptanoic acid;
3-{[((1R)-1-acetyloxy-2-methylpropoxy)carbonylamino]methyl}(3S,5R)-5-methylheptanoic acid;
3-{[((1S)-1-acetyloxy-2-methylpropoxy)carbonylamino]methyl}(3S,5R)-5-methylheptanoic acid;
3-{[((1R)-2-methyl-1-propanoyloxypropoxy)carbonylamino]methyl}(3S,5R)-5-methylheptanoic acid;
3-{[((1S)-2-methyl-1-propanoyloxypropoxy)carbonylamino]methyl}(3S,5R)-5-methylheptanoic acid;
3-({[(1R)-2-methyl-1-(2-methylpropanoyloxy)propoxy]carbonylamino}methyl)(3S,5R)-5-methylheptanoic acid;
3-({[(1S)-2-methyl-1-(2-methylpropanoyloxy)propoxy]carbonylamino}methyl)(3S,5R)-5-methylheptanoic acid; and
a pharmaceutically acceptable salt of any of the foregoing.

8. The compound of claim 1, wherein the compound is selected from:
3-{[((1R)-1-acetyloxyethoxy)carbonylamino]methyl}(3R,4R,5R)-4,5-dimethylheptanoic acid;
3-{[((1S)-1-acetyloxyethoxy)carbonylamino]methyl}(3R,4R,5R)-4,5-dimethylheptanoic acid;
3-{[((1R)-1-propanoyloxyethoxy)carbonylamino]methyl}(3R,4R,5R)-4,5-dimethylheptanoic acid;
3-{[((1S)-1-propanoyloxyethoxy)carbonylamino]methyl}(3R,4R,5R)-4,5-dimethylheptanoic acid;
3-({[(1R)-1-(2-methylpropanoyloxy)ethoxy]carbonylamino}methyl)(3R,4R,5R)-4,5-dimethylheptanoic acid;
3-({[(1S)-1-(2-methylpropanoyloxy)ethoxy]carbonylamino}methyl)(3R,4R,5R)-4,5-dimethylheptanoic acid;
3-{[((1R)-1-acetyloxy-2-methylpropoxy)carbonylamino]methyl}(3R,4R,5R)-4,5-dimethylheptanoic acid;
3-{[((1S)-1-acetyloxy-2-methylpropoxy)carbonylamino]methyl}(3R,4R,5R)-4,5-dimethylheptanoic acid;
3-{[((1R)-2-methyl-1-propanoyloxypropoxy)carbonylamino]methyl}(3R,4R,5R)-4,5-dimethylheptanoic acid;
3-{[((1S)-2-methyl-1-propanoyloxypropoxy)carbonylamino]methyl}(3R,4R,5R)-4,5-dimethylheptanoic acid;
3-({[(1R)-2-methyl-1-(2-methylpropanoyloxy)propoxy]carbonylamino}methyl)(3R,4R,5R)-4,5-dimethylheptanoic acid;
3-({[(1S)-2-methyl-1-(2-methylpropanoyloxy)propoxy]carbonylamino}methyl)(3R,4R,5R)-4,5-dimethylheptanoic acid; and
a pharmaceutically acceptable salt of any of the foregoing.

9. The compound of claim 1, wherein the compound is selected from:
3-{[((1R)-1-acetyloxyethoxy)carbonylamino]methyl}(3S,5R)-5-methyloctanoic acid;
3-{[((1S)-1-acetyloxyethoxy)carbonylamino]methyl}(3S,5R)-5-methyloctanoic acid;
3-{[((1R)-1-propanoyloxyethoxy)carbonylamino]methyl}(3S,5R)-5-methyloctanoic acid;
3-{[((1S)-1-propanoyloxyethoxy)carbonylamino]methyl}(3S,5R)-5-methyloctanoic acid;
3-({[(1R)-1-(2-methylpropanoyloxy)ethoxy]carbonylamino}methyl)(3S,5R)-5-methyloctanoic acid;
3-({[(1S)-1-(2-methylpropanoyloxy)ethoxy]carbonylamino}methyl)(3S,5R)-5-methyloctanoic acid;
3-{[((1R)-1-acetyloxy-2-methylpropoxy)carbonylamino]methyl}(3S,5R)-5-methyloctanoic acid;
3-{[((1S)-1-acetyloxy-2-methylpropoxy)carbonylamino]methyl}(3S,5R)-5-methyloctanoic acid;
3-{[((1R)-2-methyl-1-propanoyloxypropoxy)carbonylamino]methyl}(3S,5R)-5-methyloctanoic acid;
3-{[((1S)-2-methyl-1-propanoyloxypropoxy)carbonylamino]methyl}(3S,5R)-5-methyloctanoic acid;
3-({[(1R)-2-methyl-1-(2-methylpropanoyloxy)propoxy]carbonylamino}methyl)(3S,5R)-5-methyloctanoic acid;
3-({[(1S)-2-methyl-1-(2-methylpropanoyloxy)propoxy]carbonylamino}methyl)(3S,5R)-5-methyloctanoic acid; and
a pharmaceutically acceptable salt of any of the foregoing.

10. The compound of claim 1, wherein the compound is selected from:
3-{[((1R)-1-acetyloxyethoxy)carbonylamino]methyl}(3R,4R,5R)-4,5-dimethyloctanoic acid;
3-{[((1S)-1-acetyloxyethoxy)carbonylamino]methyl}(3R,4R,5R)-4,5-dimethyloctanoic acid;
3-{[((1R)-1-propanoyloxyethoxy)carbonylamino]methyl}(3R,4R,5R)-4,5-dimethyloctanoic acid;
3-{[((1S)-1-propanoyloxyethoxy)carbonylamino]methyl}(3R,4R,5R)-4,5-dimethyloctanoic acid;
3-({[(1R)-1-(2-methylpropanoyloxy)ethoxy]carbonylamino}methyl)(3R,4R,5R)-4,5-dimethyloctanoic acid;
3-({[(1S)-1-(2-methylpropanoyloxy)ethoxy]carbonylamino}methyl)(3R,4R,5R)-4,5-dimethyloctanoic acid;
3-{[((1R)-1-acetyloxy-2-methylpropoxy)carbonylamino]methyl}(3R,4R,5R)-4,5-dimethyloctanoic acid;
3-{[((1S)-1-acetyloxy-2-methylpropoxy)carbonylamino]methyl}(3R,4R,5R)-4,5-dimethyloctanoic acid;
3-{[((1R)-2-methyl-1-propanoyloxypropoxy)carbonylamino]methyl}(3R,4R,5R)-4,5-dimethyloctanoic acid;
3-{[((1S)-2-methyl-1-propanoyloxypropoxy)carbonylamino]methyl}(3R,4R,5R)-4,5-dimethyloctanoic acid;
3-({[(1R)-2-methyl-1-(2-methylpropanoyloxy)propoxy]carbonylamino}methyl)(3R,4R,5R)-4,5-dimethyloctanoic acid;
3-({[(1S)-2-methyl-1-(2-methylpropanoyloxy)propoxy]carbonylamino}methyl)(3R,4R,5R)-4,5-dimethyloctanoic acid; and
a pharmaceutically acceptable salt of any of the foregoing.

11. The compound of claim 1, wherein the compound is selected from:
3-{[((1R)-1-acetyloxyethoxy)carbonylamino]methyl}(3S,5R)-5-methylnonanoic acid;
3-{[((1S)-1-acetyloxyethoxy)carbonylamino]methyl}(3S,5R)-5-methylnonanoic acid;
3-{[((1R)-1-propanoyloxyethoxy)carbonylamino]methyl}(3S,5R)-5-methylnonanoic acid;
3-{[((1S)-1-propanoyloxyethoxy)carbonylamino]methyl}(3S,5R)-5-methylnonanoic acid;
3-({[(1R)-1-(2-methylpropanoyloxy)ethoxy]carbonylamino}methyl)(3S,5R)-5-methylnonanoic acid;
3-({[(1S)-1-(2-methylpropanoyloxy)ethoxy]carbonylamino}methyl)(3S,5R)-5-methylnonanoic acid;
3-{[((1R)-1-acetyloxy-2-methylpropoxy)carbonylamino]methyl}(3S,5R)-5-methylnonanoic acid;
3-{[((1S)-1-acetyloxy-2-methylpropoxy)carbonylamino]methyl}(3S,5R)-5-methylnonanoic acid;
3-{[((1R)-2-methyl-1-propanoyloxypropoxy)carbonylamino]methyl}(3S,5R)-5-methylnonanoic acid;
3-{[((1S)-2-methyl-1-propanoyloxypropoxy)carbonylamino]methyl}(3S,5R)-5-methylnonanoic acid;
3-({[(1R)-2-methyl-1-(2-methylpropanoyloxy)propoxy]carbonylamino}methyl)(3S,5R)-5-methylnonanoic acid;
3-({[(1S)-2-methyl-1-(2-methylpropanoyloxy)propoxy]carbonylamino}methyl)(3S,5R)-5-methylnonanoic acid; and
a pharmaceutically acceptable salt of any of the foregoing.

12. The compound of claim 1, wherein the compound is selected from:
3-{[((1R)-1-acetyloxyethoxy)carbonylamino]methyl}(3R,4R,5R)-4,5-dimethylnonanoic acid;
3-{[((1S)-1-acetyloxyethoxy)carbonylamino]methyl}(3R,4R,5R)-4,5-dimethylnonanoic acid;
3-{[((1R)-1-propanoyloxyethoxy)carbonylamino]methyl}(3R,4R,5R)-4,5-dimethylnonanoic acid;
3-{[((1S)-1-propanoyloxyethoxy)carbonylamino]methyl}(3R,4R,5R)-4,5-dimethylnonanoic acid;
3-({[(1R)-1-(2-methylpropanoyloxy)ethoxy]carbonylamino}methyl)(3R,4R,5R)-4,5-dimethylnonanoic acid;
3-({[(1S)-1-(2-methylpropanoyloxy)ethoxy]carbonylamino}methyl)(3R,4R,5R)-4,5-dimethylnonanoic acid;
3-{[((1R)-1-acetyloxy-2-methylpropoxy)carbonylamino]methyl}(3R,4R,5R)-4,5-dimethylnonanoic acid;
3-{[((1S)-1-acetyloxy-2-methylpropoxy)carbonylamino]methyl}(3R,4R,5R)-4,5-dimethylnonanoic acid;
3-{[((1R)-2-methyl-1-propanoyloxypropoxy)carbonylamino]methyl}(3R,4R,5R)-4,5-dimethylnonanoic acid;
3-{[((1S)-2-methyl-1-propanoyloxypropoxy)carbonylamino]methyl}(3R,4R,5R)-4,5-dimethylnonanoic acid;
3-({[(1R)-2-methyl-1-(2-methylpropanoyloxy)propoxy]carbonylamino}methyl)(3R,4R,5R)-4,5-dimethylnonanoic acid;
3-({[(1S)-2-methyl-1-(2-methylpropanoyloxy)propoxy]carbonylamino}methyl)(3R,4R,5R)-4,5-dimethylnonanoic acid; and
a pharmaceutically acceptable salt of any of the foregoing.

13. The compound of claim 1, wherein the compound is selected from:
3-{[((1R)-1-acetyloxyethoxy)carbonylamino]methyl}(3S,5R)-5-methyldecanoic acid;
3-{[((1S)-1-acetyloxyethoxy)carbonylamino]methyl}(3S,5R)-5-methyldecanoic acid;
3-{[((1R)-1-propanoyloxyethoxy)carbonylamino]methyl}(3S,5R)-5-methyldecanoic acid;
3-{[((1S)-1-propanoyloxyethoxy)carbonylamino]methyl}(3S,5R)-5-methyldecanoic acid;
3-({[(1R)-1-(2-methylpropanoyloxy)ethoxy]carbonylamino}methyl)(3S,5R)-5-methyldecanoic acid;
3-({[(1S)-1-(2-methylpropanoyloxy)ethoxy]carbonylamino}methyl)(3S,5R)-5-methyldecanoic acid;
3-{[((1R)-1-acetyloxy-2-methylpropoxy)carbonylamino]methyl}(3S,5R)-5-methyldecanoic acid;
3-{[((1S)-1-acetyloxy-2-methylpropoxy)carbonylamino]methyl}(3S,5R)-5-methyldecanoic acid;
3-{[((1R)-2-methyl-1-propanoyloxypropoxy)carbonylamino]methyl}(3S,5R)-5-methyldecanoic acid;
3-{[((1S)-2-methyl-1-propanoyloxypropoxy)carbonylamino]methyl}(3S,5R)-5-methyldecanoic acid;
3-({[(1R)-2-methyl-1-(2-methylpropanoyloxy)propoxy]carbonylamino}methyl)(3S,5R)-5-methyldecanoic acid;
3-({[(1S)-2-methyl-1-(2-methylpropanoyloxy)propoxy]carbonylamino}methyl)(3S,5R)-5-methyldecanoic acid; and
a pharmaceutically acceptable salt of any of the foregoing.

14. The compound of claim 1, wherein the compound is selected from:
3-{[((1R)-1-acetyloxyethoxy)carbonylamino]methyl}(3S,5R)-5-methylundecanoic acid;
3-{[((1S)-1-acetyloxyethoxy)carbonylamino]methyl}(3S,5R)-5-methylundecanoic acid;
3-{[((1R)-1-propanoyloxyethoxy)carbonylamino]methyl}(3S,5R)-5-methylundecanoic acid;
3-{[((1S)-1-propanoyloxyethoxy)carbonylamino]methyl}(3S,5R)-5-methylundecanoic acid;
3-({[(1R)-1-(2-methylpropanoyloxy)ethoxy]carbonylamino}methyl)(3S,5R)-5-methylundecanoic acid;
3-({[(1S)-1-(2-methylpropanoyloxy)ethoxy]carbonylamino}methyl)(3S,5R)-5-methylundecanoic acid;
3-{[((1R)-1-acetyloxy-2-methylpropoxy)carbonylamino]methyl}(3S,5R)-5-methylundecanoic acid;
3-{[((1S)-1-acetyloxy-2-methylpropoxy)carbonylamino]methyl}(3S,5R)-5-methylundecanoic acid;
3-{[((1R)-2-methyl-1-propanoyloxypropoxy)carbonylamino]methyl}(3S,5R)-5-methylundecanoic acid;
3-{[((1S)-2-methyl-1-propanoyloxypropoxy)carbonylamino]methyl}(3S,5R)-5-methylundecanoic acid;
3-({[(1R)-2-methyl-1-(2-methylpropanoyloxy)propoxy]carbonylamino}methyl)(3S,5R)-5-methylundecanoic acid;
3-({[(1S)-2-methyl-1-(2-methylpropanoyloxy)propoxy]carbonylamino}methyl)(3S,5R)-5-methylundecanoic acid; and
a pharmaceutically acceptable salt of any of the foregoing.

15. A compound of Formula (VII): 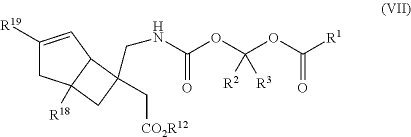
isomers thereof, and pharmaceutically acceptable salts of any of the foregoing, wherein:
R¹ is selected from C₁₋₈ acyl, substituted C₁₋₈ acyl, C₁₋₈ alkyl, substituted C₁₋₈ alkyl, C₆₋₁₀ aryl, substituted C₆₋₁₀ aryl, C₇₋₁₈ arylalkyl, substituted C₇₋₁₈ arylalkyl, C₃₋₈ cycloalkyl, substituted C₃₋₈ cycloalkyl, C₃₋₈ heterocycloalkyl, substituted C₃₋₈ heterocycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl;
R² and R³ are independently selected from hydrogen, C₁₋₈ alkyl, substituted C₁₋₈ alkyl, C₁₋₈ alkoxycarbonyl, substituted C₁₋₈ alkoxycarbonyl, C₆₋₁₀ aryl, substituted C₆₋₁₀ aryl, C₇₋₁₈ arylalkyl, substituted C₇₋₁₈ arylalkyl, carbamoyl, substituted carbamoyl, C₃₋₈ cycloalkyl, substituted C₃₋₈ cycloalkyl, C₁₋₈ heteroalkyl, substituted C₁₋₈ heteroalkyl, C₅₋₁₀ heteroaryl, substituted C₅₋₁₀ heteroaryl, C₅₋₁₀ heteroarylalkyl, and substituted C₅₋₁₀ heteroarylalkyl; or R² and R³ together with the carbon atom to which they are bonded form a ring selected from a C₃₋₈ cycloalkyl, substituted C₃₋₈ cycloalkyl, C₃₋₈ heterocycloalkyl, and substituted C₃₋₈ heterocycloalkyl ring;
R¹² is selected from hydrogen and C₁₋₆ alkyl; and
R¹⁸ and R¹⁹ are independently selected from hydrogen and C₁₋₆ alkyl.

16. The compound of claim 15, wherein R¹² is hydrogen; and R¹⁸ and R¹⁹ are independently selected from hydrogen, methyl, and ethyl.

17. The compound of claim 15, wherein each of R² and R¹² is hydrogen; R³ is selected from hydrogen, methyl, ethyl, propyl, and isopropyl; and R¹ is selected from methyl, ethyl, propyl, isopropyl, butyl, isobutyl, sec-butyl, pentyl, isopentyl, sec-pentyl, neopentyl, phenyl, 4-methoxyphenyl, benzyl, phenethyl, cyclopropyl, cyclobutyl, cyclopentyl, cyclohexyl, and 3-pyridyl.

18. The compound of claim 15, wherein each of R² and R¹² is hydrogen; R³ is selected from hydrogen, methyl, ethyl, propyl, and isopropyl; and R¹ is selected from methyl, ethyl, propyl, isopropyl, phenyl, benzyl, phenethyl, cyclopentyl, cyclohexyl, and 3-pyridyl.

19. The compound of claim 15, wherein each of R² and R¹² is hydrogen; R³ is selected from hydrogen, methyl, ethyl, propyl, and isopropyl; and R¹ is selected from methyl, ethyl, propyl, and isopropyl.

20. The compound of claim 15, wherein the moiety: 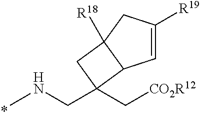
is derived from an alpha-2-delta ligand of the formula: 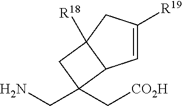
by replacing a hydrogen of the amine group with a covalent bond and by replacing the hydrogen atom of the carboxylic acid group with —R¹².

21. The compound of claim 20, wherein R¹² is hydrogen; and R¹⁸ and R¹⁹ are independently selected from hydrogen, methyl, and ethyl.

22. The compound of claim 20, wherein the alpha-2-delta ligand is selected from:
(1S,5R,6R)-6-(aminomethyl)-bicyclo[3.2.0]hept-3-ene-6-acetic acid;
(1R,5S,6S)-6-(aminomethyl)-3-methyl-bicyclo[3.2.0]hept-3-ene-6-acetic acid;
(1R,5S,6S)-6-(aminomethyl)-3-ethyl-bicyclo[3.2.0]hept-3-ene-6-acetic acid;
(1S,5R,6R)-6-(aminomethyl)-3-methyl-bicyclo[3.2.0]hept-3-ene-6-acetic acid;
(1S,5R,6R)-6-(aminomethyl)-3-ethyl-bicyclo[3.2.0]hept-3-ene-6-acetic acid; and
(1S,5R,6R)-6-(aminomethyl)-1-ethyl-bicyclo[3.2.0]hept-3-ene-6-acetic acid.

23. The compound of claim 15, wherein the compound is selected from:
2-(6-{[((1R)-1-(1-acetyloxyethoxy)carbonylamino]methyl}(1S,5R,6R)-bicyclo[3.2.0]hept-3-en-6-yl)acetic acid;
2-(6-{[((1S)-1-(1-acetyloxyethoxy)carbonylamino]methyl}(1S,5R,6R)-bicyclo[3.2.0]hept-3-en-6-yl)acetic acid;
2-(6-{[((1R)-1-(1-propanoyloxy)ethoxy)carbonylamino]methyl}(1S,5R,6R)-bicyclo[3.2.0]hept-3-en-6-yl)acetic acid;
2-(6-{[((1S)-1-(1-propanoyloxy)ethoxy)carbonylamino]methyl}(1S,5R,6R)-bicyclo[3.2.0]hept-3-en-6-yl)acetic acid;
2-[6-({[(1R)-1-(2-methylpropanoyloxy)ethoxy]carbonylamino}methyl)(1S,5R,6R)-bicyclo[3.2.0]hept-3-en-6-yl]acetic acid;
2-[6-({[(1S)-1-(2-methylpropanoyloxy)ethoxy]carbonylamino}methyl)(1S,5R,6R)-bicyclo[3.2.0]hept-3-en-6-yl]acetic acid;
2-(6-{[((1R)-1-acetyloxy-2-methylpropoxy)carbonylamino]methyl}(1S,5R,6R)-bicyclo[3.2.0]hept-3-en-6-yl]acetic acid;
2-(6-{[((1S)-1-acetyloxy-2-methylpropoxy)carbonylamino]methyl}(1S,5R,6R)-bicyclo[3.2.0]hept-3-en-6-yl]acetic acid;
2-(6-{[((1R)-2-methyl-1-propanoyloxypropoxy)carbonylamino]methyl}(1S,5R,6R)-bicyclo[3.2.0]hept-3-en-6-yl]acetic acid;
2-(6-{[((1S)-2-methyl-1-propanoyloxypropoxy)carbonylamino]methyl}(1S,5R,6R)-bicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-bicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-bicyclo[3.2.0]hept-3-en-6-yl]acetic acid; and
a pharmaceutically acceptable salt of any of the foregoing.

24. The compound of claim 15, wherein the compound is selected from:
2-(6-{[((1R)-1-(1-acetyloxyethoxy)carbonylamino]methyl}(1R,5S,6S)-3-methylbicyclo[3.2.0]hept-3-en-6-yl)acetic acid;
2-(6-{[((1S)-1-(1-acetyloxyethoxy)carbonylamino]methyl}(1R,5S,6S)-3-methylbicyclo[3.2.0]hept-3-en-6-yl)acetic acid;
2-(6-{[((1R)-1-(1-propanoyloxy)ethoxy)carbonylamino]methyl}(1R,5S,6S)-3-methylbicyclo[3.2.0]hept-3-en-6-yl)acetic acid;
2-(6-{[((1S)-1-(1-propanoyloxy)ethoxy)carbonylamino]methyl}(1R,5S,6S)-3-methylbicyclo[3.2.0]hept-3-en-6-yl)acetic acid;
2-[6-({[(1R)-1-(2-methylpropanoyloxy)ethoxy]carbonylamino}methyl)(1R,5S,6S)-3-methylbicyclo[3.2.0]hept-3-en-6-yl]acetic acid;
2-[6-({[(1S)-1-(2-methylpropanoyloxy)ethoxy]carbonylamino}methyl)(1R,5S,6S)-3-methylbicyclo[3.2.0]hept-3-en-6-yl]acetic acid;
2-(6-{[((1R)-1-acetyloxy-2-methylpropoxy)carbonylamino]methyl}(1R,5S,6S)-3-methylbicyclo[3.2.0]hept-3-en-6-yl]acetic acid;
2-(6-{[((1S)-1-acetyloxy-2-methylpropoxy)carbonylamino]methyl}(1R,5S,6S)-3-methylbicyclo[3.2.0]hept-3-en-6-yl]acetic acid;
2-(6-{[((1R)-2-methyl-1-propanoyloxypropoxy)carbonylamino]methyl}(1R,5S,6S)-3-methylbicyclo[3.2.0]hept-3-en-6-yl]acetic acid;
2-(6-{[((1S)-2-methyl-1-propanoyloxypropoxy)carbonylamino]methyl}(1R,5S,6S)-3-methylbicyclo[3.2.0]hept-3-en-6-yl]acetic acid;
2-[6-({[(1R)-2-methyl-1-(2-methylpropanoyloxy)propoxy]carbonylamino}methyl)(1R,5S,6S)-3-methylbicyclo[3.2.0]hept-3-en-6-yl]acetic acid;
2-[6-({[(1S)-2-methyl-1-(2-methylpropanoyloxy)propoxy]carbonylamino}methyl)(1R,5S,6S)-3-methylbicyclo[3.2.0]hept-3-en-6-yl]acetic acid; and
a pharmaceutically acceptable salt of any of the foregoing.

25. The compound of claim 15, wherein the compound is selected from:
2-(6-{[((1R)-1-(1-acetyloxyethoxy)carbonylamino]methyl}(1R,5S,6S)-3-ethylbicyclo[3.2.0]hept-3-en-6-yl)acetic acid;
2-(6-{[((1S)-1-(1-acetyloxyethoxy)carbonylamino]methyl}(1R,5S,6S)-3-ethylbicyclo[3.2.0]hept-3-en-6-yl)acetic acid;
2-(6-{[((1R)-1-(1-propanoyloxy)ethoxy)carbonylamino]methyl}(1R,5S,6S)-3-ethylbicyclo[3.2.0]hept-3-en-6-yl)acetic acid;
2-(6-{[((1S)-1-(1-propanoyloxy)ethoxy)carbonylamino]methyl}(1R,5S,6S)-3-ethylbicyclo[3.2.0]hept-3-en-6-yl)acetic acid;
2-[6-({[(1R)-1-(2-ethylpropanoyloxy)ethoxy]carbonylamino}methyl)(1R,5S,6S)-3-ethylbicyclo[3.2.0]hept-3-en-6-yl]acetic acid;
2-[6-({[(1S)-1-(2-ethylpropanoyloxy)ethoxy]carbonylamino}methyl)(1R,5S,6S)-3-ethylbicyclo[3.2.0]hept-3-en-6-yl]acetic acid;
2-(6-{[((1R)-1-acetyloxy-2-methylpropoxy)carbonylamino]methyl}(1R,5S,6S)-3-ethylbicyclo[3.2.0]hept-3-en-6-yl]acetic acid;
2-(6-{[((1S)-1-acetyloxy-2-methylpropoxy)carbonylamino]methyl}(1R,5S,6S)-3-ethylbicyclo[3.2.0]hept-3-en-6-yl]acetic acid;
2-(6-{[((1R)-2-methyl-1-propanoyloxypropoxy)carbonylamino]methyl}(1R,5S,6S)-3-ethylbicyclo[3.2.0]hept-3-en-6-yl]acetic acid;
2-(6-{[((1S)-2-methyl-1-propanoyloxypropoxy)carbonylamino]methyl}(1R,5S,6S)-3-ethylbicyclo[3.2.0]hept-3-en-6-yl]acetic acid;
2-[6-({[(1R)-2-methyl-1-(2-methylpropanoyloxy)propoxy]carbonylamino}methyl)(1R,5S,6S)-3-ethylbicyclo[3.2.0]hept-3-en-6-yl]acetic acid;
2-[6-({[(1S)-2-methyl-1-(2-methylpropanoyloxy)propoxy]carbonylamino}methyl)(1R,5S,6S)-3-ethylbicyclo[3.2.0]hept-3-en-6-yl]acetic acid; and
a pharmaceutically acceptable salt of any of the foregoing.

26. The compound of claim 15, wherein the compound is selected from:
2-(6-{[((1R)-1-(1-acetyloxyethoxy)carbonylamino]methyl}(1S,5R,6R)-3-methylbicyclo[3.2.0]hept-3-en-6-yl)acetic acid;
2-(6-{[((1S)-1-(1-acetyloxyethoxy)carbonylamino]methyl}(1S,5R,6R)-3-methylbicyclo[3.2.0]hept-3-en-6-yl)acetic acid;
2-(6-{[((1R)-1-(1-propanoyloxy)ethoxy)carbonylamino]methyl}(1S,5R,6R)-3-methylbicyclo[3.2.0]hept-3-en-6-yl)acetic acid;
2-(6-{[((1S)-1-(1-propanoyloxy)ethoxy)carbonylamino]methyl}(1S,5R,6R)-3-methylbicyclo[3.2.0]hept-3-en-6-yl)acetic acid;
2-[6-({[(1R)-1-(2-methylpropanoyloxy)ethoxy]carbonylamino}methyl)(1S,5R,6R)-3-methylbicyclo[3.2.0]hept-3-en-6-yl]acetic acid;
2-[6-({[(1S)-1-(2-methylpropanoyloxy)ethoxy]carbonylamino}methyl)(1S,5R,6R)-3-methylbicyclo[3.2.0]hept-3-en-6-yl]acetic acid;
2-(6-{[((1R)-1-acetyloxy-2-methylpropoxy)carbonylamino]methyl}(1S,5R,6R)-3-methylbicyclo[3.2.0]hept-3-en-6-yl]acetic acid;
2-(6-{[((1S)-1-acetyloxy-2-methylpropoxy)carbonylamino]methyl}(1S,5R,6R)-3-methylbicyclo[3.2.0]hept-3-en-6-yl]acetic acid;
2-(6-{[((1R)-2-methyl-1-propanoyloxypropoxy)carbonylamino]methyl}(1S,5R,6R)-3-methylbicyclo[3.2.0]hept-3-en-6-yl]acetic acid;
2-(6-{[((1S)-2-methyl-1-propanoyloxypropoxy)carbonylamino]methyl}(1S,5R,6R)-3-methylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-3-methylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-3-methylbicyclo[3.2.0]hept-3-en-6-yl]acetic acid; and
a pharmaceutically acceptable salt of any of the foregoing.

27. The compound of claim 15, wherein the compound is selected from:
2-(6-{[((1R)-1-(1-acetyloxyethoxy)carbonylamino]methyl}(1S,5R,6R)-3-ethylbicyclo[3.2.0]hept-3-en-6-yl)acetic acid;
2-(6-{[((1S)-1-(1-acetyloxyethoxy)carbonylamino]methyl}(1S,5R,6R)-3-ethylbicyclo[3.2.0]hept-3-en-6-yl)acetic acid;
2-(6-{[((1R)-1-(1-propanoyloxy)ethoxy)carbonylamino]methyl}(1S,5R,6R)-3-ethylbicyclo[3.2.0]hept-3-en-6-yl)acetic acid;
2-(6-{[((1S)-1-(1-propanoyloxy)ethoxy)carbonylamino]methyl}(1S,5R,6R)-3-ethylbicyclo[3.2.0]hept-3-en-6-yl)acetic acid;
2-[6-({[(1R)-1-(2-methylpropanoyloxy)ethoxy]carbonylamino}methyl)(1S,5R,6R)-3-ethylbicyclo[3.2.0]hept-3-en-6-yl]acetic acid;
2-[6-({[(1S)-1-(2-methylpropanoyloxy)ethoxy]carbonylamino}methyl)(1S,5R,6R)-3-ethylbicyclo[3.2.0]hept-3-en-6-yl]acetic acid;
2-(6-{[((1R)-1-acetyloxy-2-methylpropoxy)carbonylamino]methyl}(1S,5R,6R)-3-ethylbicyclo[3.2.0]hept-3-en-6-yl]acetic acid;
2-(6-{[((1S)-1-acetyloxy-2-methylpropoxy)carbonylamino]methyl}(1S,5R,6R)-3-ethylbicyclo[3.2.0]hept-3-en-6-yl]acetic acid;
2-(6-{[((1R)-2-methyl-1-propanoyloxypropoxy)carbonylamino]methyl}(1S,5R,6R)-3-ethylbicyclo[3.2.0]hept-3-en-6-yl]acetic acid;
2-(6-{[((1S)-2-methyl-1-propanoyloxypropoxy)carbonylamino]methyl}(1S,5R,6R)-3-ethylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-3-ethylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-3-ethylbicyclo[3.2.0]hept-3-en-6-yl]acetic acid; and
a pharmaceutically acceptable salt of any of the foregoing.

28. The compound of claim 15, wherein the compound is selected from:
2-(6-{[((1R)-1-(1-acetyloxyethoxy)carbonylamino]methyl}(1S,5R,6R)-1-ethylbicyclo[3.2.0]hept-3-en-6-yl)acetic acid;
2-(6-{[((1S)-1-(1-acetyloxyethoxy)carbonylamino]methyl}(1S,5R,6R)-1-ethylbicyclo[3.2.0]hept-3-en-6-yl)acetic acid;
2-(6-{[((1R)-1-(1-propanoyloxy)ethoxy)carbonylamino]methyl}(1S,5R,6R)-1-ethylbicyclo[3.2.0]hept-3-en-6-yl)acetic acid;
2-(6-{[((1S)-1-(1-propanoyloxy)ethoxy)carbonylamino]methyl}(1S,5R,6R)-1-ethylbicyclo[3.2.0]hept-3-en-6-yl)acetic acid;
2-[6-({[(1R)-1-(2-methylpropanoyloxy)ethoxy]carbonylamino}methyl)(1S,5R,6R)-1-ethylbicyclo[3.2.0]hept-3-en-6-yl]acetic acid;
2-[6-({[(1S)-1-(2-methylpropanoyloxy)ethoxy]carbonylamino}methyl)(1S,5R,6R)-1-ethylbicyclo[3.2.0]hept-3-en-6-yl]acetic acid;
2-(6-{[((1R)-1-acetyloxy-2-methylpropoxy)carbonylamino]methyl}(1S,5R,6R)-1-ethylbicyclo[3.2.0]hept-3-en-6-yl]acetic acid;
2-(6-{[((1S)-1-acetyloxy-2-methylpropoxy)carbonylamino]methyl}(1S,5R,6R)-1-ethylbicyclo[3.2.0]hept-3-en-6-yl]acetic acid;
2-(6-{[((1R)-2-methyl-1-propanoyloxypropoxy)carbonylamino]methyl}(1S,5R,6R)-1-ethylbicyclo[3.2.0]hept-3-en-6-yl]acetic acid;
2-(6-{[((1S)-2-methyl-1-propanoyloxypropoxy)carbonylamino]methyl}(1S,5R,6R)-1-ethylbicyclo[3.2.0]hept-3-en-6-yl]acetic acid;
2-[6-({[(1R)-2-methyl-1-(2-methylpropanoyloxy)propoxy]carbonylamino}methyl)(1S,5R,6R)-1-ethylbicyclo[3.2.0]hept-3-en-6-yl]acetic acid;
2-[6-({[(1S)-2-methyl-1-(2-methylpropanoyloxy)propoxy]carbonylamino}methyl)(1S,5R,6R)-1-ethylbicyclo[3.2.0]hept-3-en-6-yl]acetic acid; and
a pharmaceutically acceptable salt of any of the foregoing.

29. A pharmaceutical composition comprising at least one pharmaceutically acceptable vehicle and a therapeutically effective amount of a compound of claim 1.

30. A pharmaceutical composition comprising at least one pharmaceutically acceptable vehicle and a therapeutically effective amount of a compound of claim 15.

31. The pharmaceutical composition of claim 29 or 30, wherein the therapeutically effective amount is an amount sufficient to treat a disease selected from epilepsy, faintness attacks, fibromyalgia, restless legs syndrome, insomnia, a sleep disorder, hypokinesia, a cranial disorder, hot flashes, essential tremor, overactive bladder, chemical dependency and addiction, withdrawal symptoms associated chemical dependency and addiction, an obsessive/compulsive disorder, migraine, spasticity, arthritis, irritable bowel syndrome, chronic pain, acute pain, neuropathic pain, vascular headache, sinus headache, an inflammatory disorder, diuresis, premenstrual syndrome, premenstrual dysphoric disorder, tinnitus, and gastritis, in a patient.

32. A method for treating a disease in a patient comprising administering to a patient in need of such treatment a therapeutically effective amount of a compound of claim 1, wherein the disease is selected from epilepsy, faintness attacks, fibromyalgia, restless legs syndrome, insomnia, a sleep disorder, hypokinesia, a cranial disorder, hot flashes, essential tremor, overactive bladder, chemical dependency and addiction, withdrawal symptoms associated chemical dependency and addiction, an obsessive/compulsive disorder, migraine, spasticity, arthritis, irritable bowel syndrome, chronic pain, acute pain, neuropathic pain, vascular headache, sinus headache, an inflammatory disorder, diuresis, premenstrual syndrome, premenstrual dysphoric disorder, tinnitus, and gastritis.

33. A method for treating a disease in a patient comprising administering to a patient in need of such treatment a therapeutically effective amount of a compound of claim 15, wherein the disease is selected from epilepsy, faintness attacks, fibromyalgia, restless legs syndrome, insomnia, a sleep disorder, hypokinesia, a cranial disorder, hot flashes, essential tremor, overactive bladder, chemical dependency and addiction, withdrawal symptoms associated chemical dependency and addiction, an obsessive/compulsive disorder, migraine, spasticity, arthritis, irritable bowel syndrome, chronic pain, acute pain, neuropathic pain, vascular headache, sinus headache, an inflammatory disorder, diuresis, premenstrual syndrome, premenstrual dysphoric disorder, tinnitus, and gastritis.

34. A method for treating a disease in a patient comprising administering to a patient in need of such treatment a pharmaceutical composition of claim 29 or 30, wherein the disease is selected from epilepsy, faintness attacks, fibromyalgia, restless legs syndrome, insomnia, a sleep disorder, hypokinesia, a cranial disorder, hot flashes, essential tremor, overactive bladder, chemical dependency and addiction, withdrawal symptoms associated chemical dependency and addiction, an obsessive/compulsive disorder, migraine, spasticity, arthritis, irritable bowel syndrome, chronic pain, acute pain, neuropathic pain, vascular headache, sinus headache, an inflammatory disorder, diuresis, premenstrual syndrome, premenstrual dysphoric disorder, tinnitus, and gastritis.
